(19) United States
(12) Patent Application Publication
(10) Pub. No.: US 2026/0265229 A1
(43) Pub. Date: Sep. 10, 2026

(54) Small Molecule Modulators of IL-17A, Methods of Making and Methods of Using Thereof

(71) Applicant: Ascletis Pharma (China) Co., Limited, Hong Kong (CN)

(72) Inventors: Bin Liang, Hangzhou (CN); Bailing Yang, Hangzhou (CN); Jinzi Jason Wu, Hangzhou (CN)

(21) Appl. No.: 19/164,971

(22) PCT Filed: Mar. 14, 2024
(86) PCT No.: PCT/CN2024/081572

§ 371 Date: Sep. 12, 2025

Related U.S. Application Data
(60) Provisional application No. 63/492,386, filed on Mar. 27, 2023.

(30) Foreign Application Priority Data
Mar. 14, 2023 (CN) 202310267210.6

(51) Int. Cl. C07D 403/14; A61K 31/4155; A61K 31/4184; A61K 31/423; A61K 31/4245; A61K 31/428; A61K 31/437; A61K 31/4439; A61K 31/496; A61K 31/4985; A61K 31/5025; A61K 31/517; A61K 31/519; A61K 31/5377; C07D 401/14; C07D 403/12; C07D 409/14; C07D 413/14; C07D 417/14; C07D 471/04; C07D 487/04

(52) U.S. Cl. CPC C07D 403/14; A61K 31/4155; A61K 31/4184; A61K 31/423; A61K 31/4245; A61K 31/428; A61K 31/437; A61K 31/4439; A61K 31/496; A61K 31/4985; A61K 31/5025; A61K 31/517; A61K 31/519; A61K 31/5377; C07D 401/14; C07D 403/12; C07D 409/14; C07D 413/14; C07D 417/14; C07D 471/04; C07D 487/04

(57) ABSTRACT

The present application describes compounds of IL-17A modulator that are useful for treating an IL-17A mediated inflammatory syndrome, disorder, or disease.

### CROSS-REFERENCE TO RELATED APPLICATIONS

The present application claims all the benefits of the Chinese patent application No. 202310267210.6 filed Mar. 14, 2023 before the China National Intellectual Property Administration and the U.S. provisional application No. 63/492,386 filed Mar. 27, 2023 before the U.S. Patent and Trademark Office, which are incorporated by reference in their entireties.

### FIELD

Disclosed herein are compounds, and pharmaceutical compositions thereof, which modulate interleukin-17A. Also disclosed herein is the therapeutic use of such compounds, for example, in treating and/or ameliorating an IL-17A mediated inflammatory syndrome, disorder, or disease.

### BACKGROUND

The family of interleukin-17 (IL-17) cytokines, comprising IL-17A through IL-17 F, promotes the maintenance of both adaptive and innate immunity. The released cytokines act through their membrane-bound IL-17 receptor (IL-17R), a family of five receptors (IL-17RA through IL-17RE), and activating the IL-17 signal pathway. Dysregulation expression of IL-17 may contribute to inflammatory and autoimmune diseases such as psoriasis, psoriatic arthritis, rheumatoid arthritis, and multiple sclerosis.

Interleukin-17A (IL-17A) is the best investigated IL17 family member. It is well established as a pro-inflammatory cytokine, which plays a pivotal role in immune and autoimmune related diseases including psoriasis, asthma, psoriatic arthritis, and rheumatoid arthritis. IL-17A forms homodimers or heterodimers with IL-17A or IL-17F and is a major cytokine mainly secreted from Th17 cells. It signals through its membrane-bound receptors, IL-17RA and IL-17RC, and modulates IL-17A signaling pathway and triggers multiple inflammatory and immune responses. Thus, IL-17A has emerged as a major topic of interest for treating inflammatory-associated diseases.

Antagonizing IL-17A/IL-17RA protein-protein interaction (PPI) was hypothesized to reduce overexaggerated inflammation in autoimmune diseases. There are several ways to block IL-17A signaling by targeting IL-17A proteins or receptors. Clinically, several monoclonal antibodies (mAbs) are already approved for different immunological diseases. While no oral small molecule IL-17A inhibitors have progressed into late stage clinical trials yet, they are in an attractive area for discovery as their development may broaden treatment options for many patients without access to biologies. Accordingly, there is a need for new small molecule IL-17A modulators (e.g., inhibitors).

### SUMMARY

One aspect of the present application relates to a compound of Formula I:

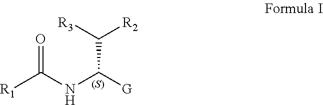

pharmaceutically acceptable salts thereof, deuterium substitutes thereof, and isomers thereof,
wherein
R₁ is selected from the group consisting of aryl, 5-6 membered heteroaryl, —C₁₋₃ alkyl-aryl and —C₁₋₃ alkyl-5-6 membered heteroaryl; wherein R₁ is optionally substituted with one or more R₁ₐ;
wherein R₁ₐ is selected from the group consisting of halo, oxo, —OH, —CN, —C₁₋₆ alkyl, and —C₀₋₂ alkyl-C₃₋₆ cycloalkyl; wherein each R₁ₐ is optionally substituted with one or more substituents each independently selected from halo;
wherein each of R₂ and R₃ is independently selected from the group consisting of H, —C₁₋₆ alkyl, aryl, C₃₋₁₀ cycloalkyl, —C₀₋₂ alkyl-C₃₋₁₀ cycloalkyl, 5-10 membered heterocyclyl, —C₀₋₂ alkyl-C₅₋₁₀ aryl and —C₀₋₂ alkyl-5-10 membered heterocyclyl; or R₂ and R₃ are combined with atoms to which they are attached to form a C₃₋₁₀ cycloalkyl or 5-10 membered heterocyclyl; wherein the C₃₋₁₀ cycloalkyl or the 5-10 membered heterocyclyl is optionally substituted with one or more substituents each independently selected from the group consisting of halo, —C₁₋₉ alkyl, and —C₁₋₈ haloalkyl;
wherein G is selected from the group consisting of

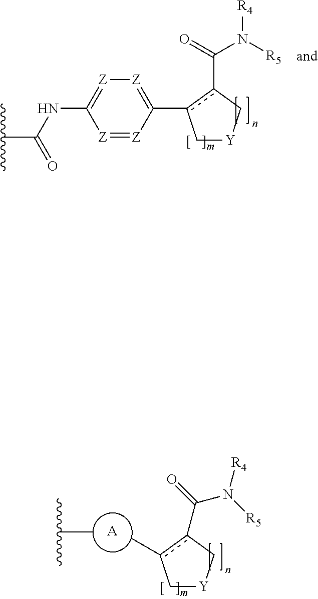

wherein m=0, 1, or 2;
n=0, 1, or 2;
wherein Z is N or —C(R₇)—;
wherein ring A is 9-10 membered heteroaryl; wherein ring A is optionally substituted with one or more substituents each independently selected from the group consisting of halogen, oxo, —OH, —CN, —C₁₋₆ alkyl, —C₁₋₆ alkoxy, —C₁₋₆ haloalkoxy, —C₃₋₁₀ cycloalkyl, heterocyclyl, —C₆₋₁₀ aryl and 5-10 membered heteroaryl;
wherein Y is selected from the group consisting of O, S, SO, S(O)₂, and

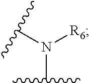

wherein each of R₄ and R₅ is independently selected from the group consisting of H, OH, —C₁₋₉ alkyl, —C₁₋₈ haloalkyl, —C₂₋₆ alkenyl, —C₂₋₆ alkynyl, —COR₆ₐ, —C₁₋₄ alkyl-C₃₋₈ cycloalkyl, —C₁₋₄ alkyl-heterocyclyl, —C₃₋₁₀ cycloalkyl, heterocyclyl, —C₆₋₁₀ aryl, 5-10 membered heteroaryl, —C₁₋₄ alkyl-SO₂—C₁₋₄ alkyl, and —C₁₋₄ alkyl-SO₂—C₃₋₈ cycloalkyl;
wherein each of R₄ and R₅ is optionally substituted with one or more substituents each independently selected from the group consisting of H, halogen, oxo, —OH, —CN, —C₁₋₆ alkyl, —C₁₋₆ alkoxy, —C₁₋₆ haloalkoxy, —C₃₋₁₀ cycloalkyl, heterocyclyl, —C₆₋₁₀ aryl, and 5-10 membered heteroaryl; or R₄ and R₅ are combined with atoms to which they are attached to form 3-10 membered heterocyclyl; wherein the 3-10 membered heterocyclyl is optionally substituted with one or more substituents each independently selected from the group consisting of halo, —C₁₋₉ alkyl, and —C₁₋₈ haloalkyl;
wherein R₆ is independently selected from the group consisting of H, C₁₋₉ alkyl, C₁₋₈ haloalkyl, —C₁₋₆ alkyl-alkoxy, —C₁₋₆ alkyl-cycloalkoxy, C₂₋₆ alkenyl, C₂₋₆ alkynyl, C₃₋₁₀ cycloalkyl, heterocyclyl, C₆₋₁₀ aryl, 5-10 membered heteroaryl, CO₂R₆ₐ, COR₆ₐ, CON(R₆ₐ)(R_{6b}), —S(O) R₆ₐ, —S(O)(NH)R₆ₐ, —S(O)₂R₆ₐ, —S(O)₂OR₆ₐ—S(O)₂N(R₆ₐ)(R_{6b}), and —S(O)(NR₆ₐ)R₆ₐ;
wherein R₆ is optionally substituted with one or more substituents each independently selected from the group consisting of halogen, oxo, —OH, —CN, —C₁₋₆ alkyl, —C₁₋₆ alkoxy, —C₁₋₆ haloalkoxy, —C₁₋₆ haloalkyl, —C₃₋₁₀ cycloalkyl, heterocyclyl, —C₆₋₁₀ aryl, and 5-10 membered heteroaryl;
wherein each of R₆ₐ and R_{6b} is independently selected from the group consisting of H, C₁₋₉ alkyl, C₁₋₈ haloalkyl, C₂₋₆ alkenyl, C₂₋₆ alkynyl, C₃₋₁₀ cycloalkyl, heterocyclyl, C₆₋₁₀ aryl, and 5-10 membered heteroaryl; or R₆ₐ and R_{6b} are combined with atoms to which they are attached to form a C₃₋₁₀ cycloalkyl or 5-10 membered heterocyclyl; wherein the C₃₋₁₀ cycloalkyl or 5-10 membered heterocyclyl is optionally substituted with one or more substituents each independently selected from the group consisting of halo, —C₁₋₉ alkyl, and —C₁₋₈ haloalkyl;
wherein each of R₆ₐ and R_{6b} is optionally substituted with one or more substituents each independently selected from the group consisting of halogen, oxo, —OH, —CN, —C₁₋₆ alkyl, —C₁₋₆ alkoxy, —C₁₋₆ haloalkoxy, —C₁₋₆ haloalkyl, —C₃₋₁₀ cycloalkyl, heterocyclyl, —C₆₋₁₀ aryl, 5-10 membered heteroaryl, —C₁₋₄ alkyl-C₃₋₈ cycloalkyl, and —C₁₋₄ alkyl-heterocyclyl;
wherein R₇ is selected from the group consisting of H, halogen, oxo, —OH, —CN, —C₁₋₉ alkyl, —C₁₋₈ haloalkyl, —C₁₋₆ alkoxy, —C₁₋₆ haloalkoxy, —C₂₋₆ alkenyl, —C₂₋₆ alkynyl, —CO₂R₇ₐ, —COR₇ₐ, —CONHR₇ₐ, —NO₂, —NH₂, —N₃, —SH, —O(C₁₋₉ alkyl), —O(C₁₋₈ haloalkyl), —NH(C₁₋₉ alkyl), —NH(C₁₋₈ haloalkyl), —N(C₁₋₉ alkyl)₂, and —N(C₁₋₈ haloalkyl)₂;
wherein R₇ₐ is selected from the group consisting of H, —C₁₋₆ alkyl, —C₁₋₆ haloalkyl, —C₁₋₄ alkyl-C₃₋₈ cycloalkyl, —C₁₋₄ alkyl-heterocyclyl, C₃₋₁₀ cycloalkyl, heterocyclyl, C₆₋₁₀ aryl, and 5-10 membered heteroaryl; and wherein the 5-10 membered heteroaryl or the heterocyclyl is optionally substituted with one or more substituents each independently selected from the group consisting of N, O, and S.

In another aspect, the present application relates to a composition comprising a compound of Formula I, pharmaceutically acceptable salts thereof, deuterium substitutes thereof, isomers thereof.

In another aspect, the present application relates to a method of modulating IL-17A, comprising administering to a subject in need thereof an effective amount of a composition comprising a compound of Formula I of the present application, a pharmaceutically acceptable salt thereof, a deuterium substitute thereof, or an isomer thereof or a composition comprising a compound of Formula I of the present application, a pharmaceutically acceptable salt thereof, a deuterium substitute thereof, or an isomer thereof.

In another aspect, the present application relates to the use of a compound of Formula I, a pharmaceutically acceptable salt thereof, a deuterium substitute thereof, or an isomer thereof in the manufacture of a medicament for modulating IL-17A.

In another aspect, the present application relates to a method of treating an inflammatory disease or condition, comprising administering to a subject in need thereof an effective amount of the pharmaceutical composition comprising a compound of Formula I of the present application, a pharmaceutically acceptable salt thereof, a deuterium substitute thereof, or an isomer thereof or a composition comprising a compound of Formula I of the present application, a pharmaceutically acceptable salt thereof, a deuterium substitute thereof, or an isomer thereof.

### DETAILED DESCRIPTION

### I. Definitions

Unless defined otherwise, all technical and scientific terms used herein have the same meaning as commonly understood by one of ordinary skill in the art. A dash at the front or end of a chemical group is a matter of convenience to indicate the point of attachment to a parent moiety; chemical groups may be depicted with or without one or more dashes without losing their ordinary meaning. A prefix such as “Cᵤ₋ᵥ” or “Cᵤ-Cᵥ” indicates that the following group has from u to v carbon atoms, where u and v are integers. For example, “C₁₋₆ alkyl” or “C₁-C₆ alkyl” indicates that the alkyl group has from 1 to 6 carbon atoms.

“Alkyl” is a monovalent or divalent linear or branched saturated hydrocarbon radical. For example, an alkyl group can have 1 to 10 carbon atoms (i.e., C₁₋₁₀ alkyl) or 1 to 8 carbon atoms (i.e., C₁₋₈ alkyl) or 1 to 6 carbon atoms (i.e., C₁₋₆ alkyl) or 1 to 4 carbon atoms (i.e., C₁₋₄ alkyl). Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-Propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)—CH₃). Alkyl groups can be unsubstituted or substituted.

The term “heteroalkyl” by itself or in combination with other terms refers to a stable linear or branched hydrocarbon alkyl group consisting of at least one heteroatom selected from N, O and S, wherein the nitrogen and sulfur atoms are optionally oxidized and nitrogen atom(s) are optionally quaternized. The heteroatom(s) can be at any internal position of the heteroalkyl group, e.g., —CH₂—CH₂—O—CH₃, —CH₂—CH₂—NH—CH₃, —CH₂—CH₂—N(CH₃)—CH, —C—S—CH₂—CH₃, —CH₂—CH₂—S(O)—CH₃, —CH₂—CH₂—S(O)₂—CH₃. heteroalkynyl Up to two heteroatoms may be consecutive, e.g., —CH₂—NH—OCH₃.

“Alkenyl” is a monovalent or divalent linear or branched hydrocarbon radical with at least one carbon-carbon double bond. For example, an alkenyl group can have 2 to 8 carbon atoms (i.e., C₂₋₈ alkenyl) or 2 to 6 carbon atoms (i.e., C₂₋₆ alkenyl) or 2 to 4 carbon atoms (i.e., C₂₋₄ alkenyl). Examples of alkenyl groups include, but are not limited to, ethenyl (—CH═CH₂), allyl (—CH₂CH═CH₂), and —CH₂—CH═CH—CH₃. Alkenyl groups can be unsubstituted or substituted.

“Alkynyl” is a monovalent or divalent linear or branched hydrocarbon radical with at least one carbon-carbon triple bond. For example, an alkynyl group can have 2 to 8 carbon atoms (i.e., C₂₋₈ alkynyl) or 2 to 6 carbon atoms (i.e., C₂₋₆ alkynyl) or 2 to 4 carbon atoms (i.e., C₂₋₄ alkynyl). Examples of alkynyl groups include, but are not limited to, acetylenyl (—C≡CH), propargyl (—CH₂C≡CH), and —CH2-C≡C—CH₃. Alkynyl groups can be unsubstituted or substituted.

“Halogen” or “Halo” refers to fluoro (—F), chloro (—Cl), bromo (—Br) and iodo (—I).

“Haloalkyl” is an alkyl as defined herein, wherein one or more hydrogen atoms of the alkyl are independently replaced by a halogen, which may be the same or different, such that the alkyl is divalent. The alkyl group and the halogen can be any of those described above. In some embodiments, the haloalkyl defines the number of carbon atoms in the alkyl portion, e.g., C₁₋₄ haloalkyl includes CF₃, CH₂F, CHF₂, CH₂CF₃, CH₂CH₂CF₃, CCl₂CH₂CH₂CH₃, and C(CH₃)₂(CF₂H). Haloalkyl groups can be unsubstituted or substituted.

“Alkoxy” refers to the group —O-alkyl, where alkyl is as defined above. For example, C₁₋₄ alkoxy refers to an —O-alkyl group having 1 to 4 carbons. Alkoxy groups can be unsubstituted or substituted.

“Haloalkoxy” is an alkoxy as defined herein, wherein one or more hydrogen atoms of the alkyl in the alkyloxy are independently replaced by a halogen, which may be the same or different, such that the alkyl is divalent. The alkoxy group and the halogen can be any of those described above. In some embodiments, the haloalkoxy defines the number of carbon atoms in the alkyl portion, e.g., C₁₋₄ haloalkoxy includes OCF₃, OCH₂F, OCH₂CF₃, OCH₂CH₂CF₃, OCCl₂CH₂CH₂CH₃, and OC(CH₃)₂ (CF₂H). Haloalkoxy groups can be unsubstituted or substituted.

“Cycloalkyl” is a monovalent or divalent single all carbon ring or a multiple condensed all carbon ring system wherein the ring in each instance is a non-aromatic saturated or unsaturated ring. For example, in some embodiments, a cycloalkyl group has 3 to 12 carbon atoms, 3 to 10 carbon atoms, 3 to 8 carbon atoms, 3 to 6 carbon atoms, 3 to 5 carbon atoms, or 3 to 4 carbon atoms. Exemplary single ring cycloalkyl groups include cyclopropyl, cyclobutyl, cyclopentyl, cyclohexyl, cyclohexenyl, cycloheptyl, cycloheptenyl, and cyclooctyl. Cycloalkyl also includes multiple condensed ring systems (e.g., ring systems comprising 2 rings) having about 7 to 12 carbon atoms. The rings of the multiple condensed ring system can be connected to each other via fused, spiro, or bridged bonds when allowed by valency requirements. Exemplary multiple ring cycloalkyl groups include octahydropentalene, bicyclo[2.2.1]heptane, bicyclo[2.2.2]octane, bicyclo[2.2.2]oct-2-ene, and spiro[2.5]octane. Cycloalkyl groups can be unsubstituted or substituted.

“Aryl” as used herein refers to a monovalent or divalent single all carbon aromatic ring or a multiple condensed all carbon ring system wherein the ring is aromatic. For example, in some embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed ring systems (e.g., ring systems comprising 2, 3 or 4 rings) having about 9 to 20 carbon atoms in which multiple rings are aromatic. The rings of the multiple condensed ring system can be connected to each other via fused bonds when allowed by valency requirements. It is also understood that when reference is made to a certain atom-range membered aryl (e.g., 6-10 membered aryl), the atom range is for the total ring atoms of the aryl. For example, a 6-membered aryl would include phenyl and a 10-membered aryl would include naphthyl. Non-limiting examples of aryl groups include, but are not limited to, phenyl, naphthyl, anthracenyl, and the like. Aryl groups can be unsubstituted or substituted.

“Alkylaryl” refers to an alkyl as defined herein, wherein one or more hydrogen atoms of the alkyl are independently replaced by an aryl group, which may be the same or different. The alkyl group and the aryl group can be any of those described above, such that the alkyl is divalent. In some embodiments, an alkylaryl group has 7 to 24 carbon atoms, 7 to 16 carbon atoms, 7 to 13 carbon atoms, or 7 to 11 carbon atoms. An alkylaryl group defined by the number of carbon atoms refers to the total number of carbon atoms present in the constitutive alkyl and aryl groups combined. For heteroalkyl example, C₇ alkylaryl refers to benzyl, while C₁₁ alkylaryl includes 1-methylnaphthyl and n-pentylphenyl. In some embodiments the number of carbon atoms in the alkyl and aryl portion can be designated separately, e.g., C₁₋₆ alkyl-C₆₋₁₀ aryl. Non-limiting examples of alkylaryl groups include, but are not limited to, benzyl, 2,2-dimethylphenyl, n-pentylphenyl, 1-methylnaphthyl, 2-ethylnaphthyl, and the like. Alkylaryl groups can be unsubstituted or substituted.

“Heterocyclyl” or “heterocycle” or “heterocycloalkyl” as used herein refers to a single saturated or partially unsaturated non-aromatic ring or a non-aromatic multiple ring system that has at least one heteroatom in the ring (i.e., at least one annular (i.e., ring-shaped) heteroatom selected from oxygen, nitrogen, and sulfur). Unless otherwise specified, a heterocyclyl group has from 3 to about 20 annular atoms, for example from 3 to 12 annular atoms, for example from 4 to 12 annular atoms, 4 to 10 annular atoms, or 3 to 8 annular atoms, or 3 to 6 annular atoms, or 3 to 5 annular atoms, or 4 to 6 annular atoms, or 4 to 5 annular atoms. Thus, the term includes single saturated or partially unsaturated rings (e.g., 3, 4, 5, 6 or 7-membered rings) having from about 1 to 6 annular carbon atoms and from about 1 to 3 annular heteroatoms selected from the group consisting of oxygen, nitrogen and sulfur in the ring. The rings of the multiple condensed ring (e.g. bicyclic heterocyclyl) system can be connected to each other via fused, spiro and bridged bonds when allowed by valency requirements. Heterocycles include, but are not limited to, azetidine, aziridine, imidazolidine, morpholine, oxirane (epoxide), oxetane, thietane, piperazine, piperidine, pyrazolidine, piperidine, pyrrolidine, pyrrolidinone, tetrahydrofuran, tetrahydrothiophene, dihydropyridine, tetrahydropyridine, quinuclidine, 2-oxa-6-azaspiro[3.3]heptan-6-yl, 6-oxa-1-azaspiro[3.3]heptan-1-yl, 2-thia-6-azaspiro[3.3]heptan-6-yl, 2,6-diazaspiro[3.3]heptan-2-yl, 2-azabicyclo[3.1.0]hexan-2-yl, 3-azabicyclo[3.1.0]hexanyl, 2-azabicyclo[2.1.1]hexanyl, 2-azabicyclo[2.2.1]heptan-2-yl, 4-azaspiro[2.4]heptanyl, 5-azaspiro[2.4]heptanyl, and the like. Heterocyclyl groups can be unsubstituted or substituted.

The term “heteroalkenyl” by itself or in combination with other terms refers to a stable linear or branched hydrocarbon alkyl group consisting of at least one heteroatom selected from N, O and S, wherein the nitrogen and sulfur atoms are optionally oxidized and nitrogen atom(s) are optionally quaternized. The heteroatom(s) can be at any internal position of the heteroalkenyl group. Up to two heteroatoms may be consecutive.

The term “heteroalkynyl” by itself or in combination with other terms refers to a stable linear or branched hydrocarbon alkynyl group consisting of at least one heteroatom selected from N, O and S, wherein the nitrogen and sulfur atoms are optionally oxidized and nitrogen atom(s) are optionally quaternized. The heteroatom(s) can be at any internal position of the heteroalkynyl group. Up to two heteroatoms may be consecutive.

“5-10 membered heteroaryl” or “heteroaryl” refers to a single aromatic ring that has at least one atom other than carbon in the ring, wherein the atom is selected from the group consisting of oxygen, nitrogen and sulfur; “5-10 membered heteroaryl” also includes multiple condensed ring systems that have at least one such aromatic ring, which multiple condensed ring systems are further described below. Thus, “5-10 membered heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary 5-10 membered heteroaryl ring systems include but are not limited to pyridyl, pyrimidinyl, oxazolyl or furyl. “5-10 membered heteroaryl” also includes multiple condensed ring systems (e.g., ring systems comprising 2, 3 or 4 rings) wherein a 5-10 membered heteroaryl group, as defined above, is condensed with one or more rings selected from 5-10 membered heteroaryls (to form for example 1,8-naphthyridinyl) and aryls (to form, for example, benzimidazolyl or indazolyl) to form the multiple condensed ring system. Thus, a 5-10 membered heteroaryl (a single aromatic ring or multiple condensed ring system) can have about 1-20 carbon atoms and about 1-6 heteroatoms within the 5-10 membered heteroaryl ring. For example, tetrazolyl has 1 carbon atom and 4 nitrogen heteroatoms within the ring. The rings of the multiple condensed ring system can be connected to each other via fused bonds when allowed by valency requirements. It is to be understood that the individual rings of the multiple condensed ring system may be connected in any order relative to one another. It is to be understood that the point of attachment for a 5-10 membered heteroaryl or 5-10 membered heteroaryl multiple condensed ring system can be at any suitable atom of the 5-10 membered heteroaryl or 5-10 membered heteroaryl multiple condensed ring system including a carbon atom and a heteroatom (e.g., a nitrogen). It also to be understood that when a reference is made to a certain atom-range membered (e.g., a 5-10 membered heteroaryl), the atom range is for the total ring atoms of the 5-10 membered heteroaryl and includes carbon atoms and heteroatoms. It is also to be understood that the rings of the multiple condensed ring system may include an aryl ring fused to a heterocyclic ring with saturated or partially unsaturated bonds (e.g., 3, 4, 5, 6 or 7-membered rings) having from about 1 to 6 annular carbon atoms and from about 1 to 3 annular heteroatoms selected from the group consisting of oxygen, nitrogen and sulfur in the ring. For example, a 5-10 membered heteroaryl includes thiazolyl and a 5-10 membered heteroaryl includes quinolinyl. Exemplary 5-10 membered heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, benzofuranyl, benzimidazolyl, thianaphthenyl, pyrrolo[2,3-b]pyridinyl, quinazolinyl-4(3H)-one, triazolyl, and tetrazolyl. 5-10 membered heteroaryl groups can be unsubstituted or substituted.

“Alkylheteroaryl” refers to an alkyl as defined herein, wherein one or more hydrogen atoms of the alkyl are independently replaced by a heteroaryl group, which may be the same or different, such that the alkyl is divalent. The alkyl group and the heteroaryl group can be any of those described above. In some embodiments, the number of atoms in the alkyl and heteroaryl portion are designated separately, e.g., C₁₋₆ alkyl-5 to 10 membered heteroaryl having one to four heteroatoms each independently N, O, or S. Alkylheteroaryl groups can be unsubstituted or substituted.

“Oxo” as used herein refers to ═O

“ [symbol] ” refers to a bond that may be a single bond or a double bond.

“Substituted” as used herein refers to wherein one or more hydrogen atoms of the group are independently replaced by one or more substituents (e.g., 1, 2, 3, or 4 or more) as indicated.

A “compound of the present application” includes compounds disclosed herein, for example a compound of the present application includes compounds of Formula I, including the compounds of the Examples. In some embodiments, a “compound of the present application” includes compounds of Formula I.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Therapeutically effective amount” or “effective amount” as used herein refers to an amount that is effective to elicit the desired biological or medical response, including the amount of a compound that, when administered to a subject for treating a disease, is sufficient to affect such treatment for the disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present application is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present application within seconds or minutes. In some embodiments, a unit dose of a compound of the present application is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present application. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the subject.

Provided are also pharmaceutically acceptable salts, hydrates, solvates, tautomeric forms, polymorphs, and prodrugs of the compounds described herein.

Pharmaceutically acceptable” or “physiologically acceptable” refer to compounds, salts, compositions, dosage forms and other materials which are useful in preparing a pharmaceutical composition that is suitable for veterinary or human pharmaceutical use.

The compounds described herein may be prepared and/or formulated as pharmaceutically acceptable salts or when appropriate as a free base. Pharmaceutically acceptable salts are non-toxic salts of a free base form of a compound that possesses the desired pharmacological activity of the free base. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington: The Science and Practice of Pharmacy, 21st Edition, Lippincott Williams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and N(C₁-C₄ alkyl) 4+. Also included are base addition salts, such as sodium or potassium salts.

Provided also are compounds described herein or pharmaceutically acceptable salts, isomers, or a mixture thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds (also referred to as “deuterium substitutes” or “deuterated compounds”) may increase resistance to metabolism, and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogen atoms have been replaced by deuterium.

Provided also are compounds described herein or pharmaceutically acceptable salts, isomers, or a mixture thereof, in which from 1 to n atoms may be replaced independently by 1 to n corresponding isotopes. Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula I-A-1 can generally be prepared by conventional techniques known to those skilled in the art or by processes analogous to those described in the Examples as set out below using an appropriate isotopically-labeled reagent in place of the non-labeled reagent previously employed.

The compounds of the embodiments disclosed herein, or their pharmaceutically acceptable salts may contain one or more asymmetric centers and may thus give rise to enantiomers, tautomer, diastereomers, and other stereoisomeric forms that may be defined, in terms of absolute stereochemistry, as (R)- or (S)- or, as (D)- or (L)- for amino acids, as well as deuterated analogs thereof. The chemical formula shown in the present application is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1

“Stereoisomer” as used herein refers to a compound made up of the same atoms bonded by the same bonds but having different three-dimensional structures, which are not interchangeable. The present application contemplates various stereoisomers and mixtures thereof and includes “enantiomers”, which refers to two stereoisomers whose molecules are non-superimposable mirror images of one another.

“Tautomer” as used herein refers to a proton shift from one atom of a molecule to another atom of the same molecule. In some embodiments, the present application includes tautomers of said compounds.

“Solvate” as used herein refers to the result of the interaction of a solvent and a compound. Solvates of salts of the compounds described herein are also provided. Hydrates of the compounds described herein are also provided.

“Hydrate” as used herein refers to a compound of the disclosure that is chemically associated with one or more molecules of water.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Prodrug” as used herein refers to a derivative of a drug that upon administration to the human body is converted to the parent drug according to some chemical or enzymatic pathway. In some embodiments, a prodrug is a biologically inactive derivative of a drug that upon administration to the human body is converted to the biologically active parent drug according to some chemical or enzymatic pathway.

“Treatment” or “treat” or “treating” as used herein refers to an approach for obtaining beneficial or desired results. For purposes of the present application, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival. “At risk individual” as used herein refers to an individual who is at risk of developing a condition to be treated. An individual “at risk” may or may not have detectable disease or condition, and may or may not have displayed detectable disease prior to the treatment of methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s).

### II. Compounds

One aspect of the present application relates to a compound of Formula I:

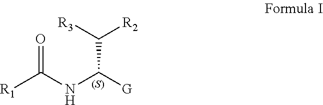

pharmaceutically acceptable salts thereof, deuterium substitutes thereof, and isomers thereof,
wherein
R₁ is selected from the group consisting of aryl, 5-6 membered heteroaryl, —C₁₋₃ alkyl-aryl and —C₁₋₃ alkyl-5-6 membered heteroaryl; wherein R₁ is optionally substituted with one or more R₁ₐ;
wherein R₁ₐ is selected from the group consisting of halo, oxo, —OH, —CN, —C₁₋₆ alkyl, and —C₀₋₂ alkyl-C₃₋₆ cycloalkyl; wherein each R₁ₐ is optionally substituted with one or more substituents each independently selected from halo;
wherein each of R₂ and R₃ is independently selected from the group consisting of H, —C₁₋₆ alkyl, aryl, C₃₋₁₀ cycloalkyl, —C₀₋₂ alkyl-C₃₋₁₀ cycloalkyl, 5-10 membered heterocyclyl, —C₀₋₂ alkyl-C₅₋₁₀ aryl and —C₀₋₂ alkyl-5-10 membered heterocyclyl; or R₂ and R₃ are combined with atoms to which they are attached to form a C₃₋₁₀ cycloalkyl or 5-10 membered heterocyclyl; wherein the C₃₋₁₀ cycloalkyl or the 5-10 membered heterocyclyl is optionally substituted with one or more substituents each independently selected from the group consisting of halo, —C₁₋₉ alkyl, and —C₁₋₈ haloalkyl;
wherein G is selected from the group consisting of

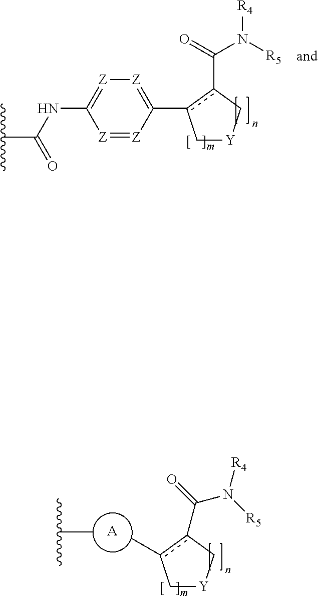

wherein m=0, 1, or 2;
n=0, 1, or 2;
wherein Z is N or —C(R₇)—;
wherein ring A is 9-10 membered heteroaryl; wherein ring A is optionally substituted with one or more substituents each independently selected from the group consisting of halogen, oxo, —OH, —CN, —C₁₋₆ alkyl, —C₁₋₆ alkoxy, —C₁₋₆ haloalkoxy, —C₃₋₁₀ cycloalkyl, heterocyclyl, —C₆₋₁₀ aryl and 5-10 membered heteroaryl;
wherein Y is selected from the group consisting of O, S, SO, S(O)₂, and

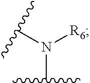

wherein each of R₄ and R₅ is independently selected from the group consisting of H, OH, —C₁₋₉ alkyl, —C₁₋₈ haloalkyl, —C₂₋₆ alkenyl, —C₂₋₆ alkynyl, —COR₆ₐ, —C₁₋₄ alkyl-C₃₋₈ cycloalkyl, —C₁₋₄ alkyl-heterocyclyl, —C₃₋₁₀ cycloalkyl, heterocyclyl, —C₆₋₁₀ aryl, 5-10 membered heteroaryl, —C₁₋₄ alkyl-SO₂—C₁₋₄ alkyl, and —C₁₋₄ alkyl-SO₂—C₃₋₈ cycloalkyl;
wherein each of R₄ and R₅ is optionally substituted with one or more substituents each independently selected from the group consisting of H, halogen, oxo, —OH, —CN, —C₁₋₆ alkyl, —C₁₋₆ alkoxy, —C₁₋₆ haloalkoxy, —C₃₋₁₀ cycloalkyl, heterocyclyl, —C₆₋₁₀ aryl, and 5-10 membered heteroaryl; or R₄ and R₅ are combined with atoms to which they are attached to form 3-10 membered heterocyclyl; wherein the 3-10 membered heterocyclyl is optionally substituted with one or more substituents each independently selected from the group consisting of halo, —C₁₋₉ alkyl, and —C₁₋₈ haloalkyl;
wherein R₆ is independently selected from the group consisting of H, C₁₋₉ alkyl, C₁₋₈ haloalkyl, —C₁₋₆ alkyl-alkoxy, —C₁₋₆ alkyl-cycloalkoxy, C₂₋₆ alkenyl, C₂₋₆ alkynyl, C₃₋₁₀ cycloalkyl, heterocyclyl, C₆₋₁₀ aryl, 5-10 membered heteroaryl, CO₂R₆ₐ, COR₆ₐ, CON(R₆ₐ)(R_{6b}), —S(O)R₆ₐ, —S(O)(NH)R₆ₐ, —S(O)₂R₆ₐ, —S(O)₂OR₆ₐ—S(O)₂N(R₆ₐ)(R_{6b}), and —S(O)(NR₆ₐ)R₆ₐ;
wherein R₆ is optionally substituted with one or more substituents each independently selected from the group consisting of halogen, oxo, —OH, —CN, —C₁₋₆ alkyl, —C₁₋₆ alkoxy, —C₁₋₆ haloalkoxy, —C₁₋₆ haloalkyl, —C₃₋₁₀ cycloalkyl, heterocyclyl, —C₆₋₁₀ aryl, and 5-10 membered heteroaryl;
wherein each of R₆ₐ and R_{6b} is independently selected from the group consisting of H, C₁₋₉ alkyl, C₁₋₈ haloalkyl, C₂₋₆ alkenyl, C₂₋₆ alkynyl, C₃₋₁₀ cycloalkyl, heterocyclyl, C₆₋₁₀ aryl, and 5-10 membered heteroaryl; or R₆ₐ and R_{6b} are combined with atoms to which they are attached to form a C₃₋₁₀ cycloalkyl or 5-10 membered heterocyclyl; wherein the C₃₋₁₀ cycloalkyl or 5-10 membered heterocyclyl is optionally substituted with one or more substituents each independently selected from the group consisting of halo, —C₁₋₉ alkyl, and —C₁₋₈ haloalkyl;
wherein each of R₆ₐ and R_{6b} is optionally substituted with one or more substituents each independently selected from the group consisting of halogen, oxo, —OH, —CN, —C₁₋₆ alkyl, —C₁₋₆ alkoxy, —C₁₋₆ haloalkoxy, —C₁₋₆ haloalkyl, —C₃₋₁₀ cycloalkyl, heterocyclyl, —C₆₋₁₀ aryl, 5-10 membered heteroaryl, —C₁₋₄ alkyl-C₃₋₈ cycloalkyl, and —C₁₋₄ alkyl-heterocyclyl;
wherein R₇ is selected from the group consisting of H, halogen, oxo, —OH, —CN, —C₁₋₉ alkyl, —C₁₋₈ haloalkyl, —C₁₋₆ alkoxy, —C₁₋₆ haloalkoxy, —C₂₋₆ alkenyl, —C₂₋₆ alkynyl, —CO₂R₇ₐ, —COR₇ₐ, —CONHR₇ₐ, —NO₂, —NH₂, —N₃, —SH, —O(C₁₋₉ alkyl), —O(C₁₋₈ haloalkyl), —NH(C₁₋₉ alkyl), —NH(C₁₋₈ haloalkyl), —N(C₁₋₉ alkyl)₂, and —N(C₁₋₈ haloalkyl)₂;
   wherein R₇ₐ is selected from the group consisting of H, —C₁₋₆ alkyl, —C₁₋₆ haloalkyl, —C₁₋₄ alkyl-C₃₋₈ cycloalkyl, —C₁₋₄ alkyl-heterocyclyl, C₃₋₁₀ cycloalkyl, heterocyclyl, C₆₋₁₀ aryl, and 5-10 membered heteroaryl; and wherein the 5-10 membered heteroaryl or the heterocyclyl is optionally substituted with one or more substituents each independently selected from the group consisting of N, O, and S.

In some embodiments, the present application relates to a compound of Formula Ia or Ib, pharmaceutically acceptable salts thereof, deuterium substitutes thereof, and isomers thereof:

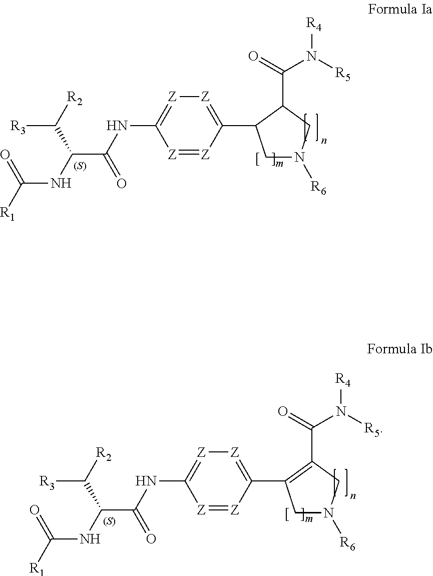

In some embodiments, the present application relates to a compound of Formula Ila or IIb, pharmaceutically acceptable salts thereof, deuterium substitutes thereof, and isomers thereof:

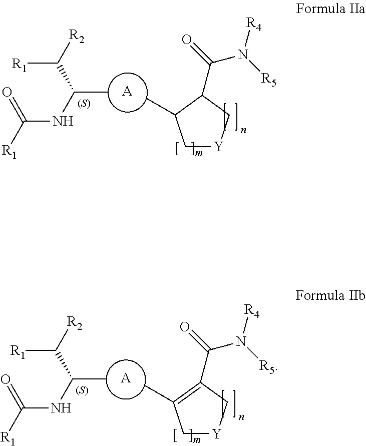

In some embodiments, ring A is selected from the group consisting of:

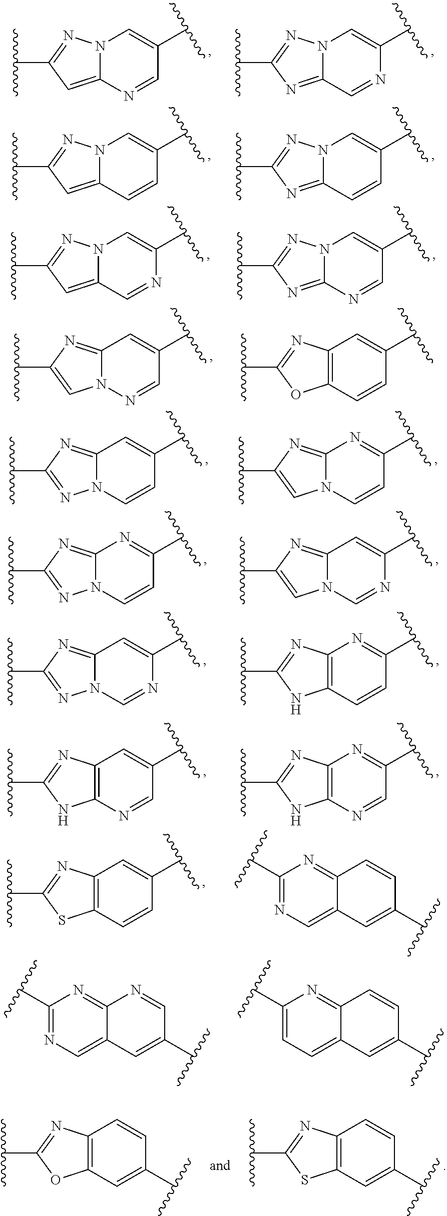

In some embodiments, the compound of the present application has a structure of Formula III:

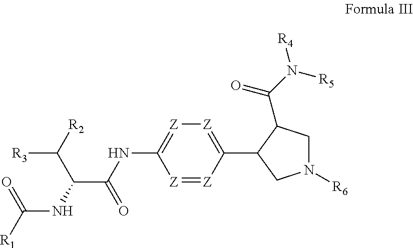

wherein R₁ is selected from the group consisting of aryl, 5-6 membered heteroaryl; wherein R₁ is optionally substituted with one or more R₁ₐ; wherein each of R₁ₐ is independently selected from the group consisting of halo-C₁₋₆ alkyl, and —C₀₋₂ alkyl-C₃₋₆ cycloalkyl; wherein each R₁ₐ is optionally substituted with one or more substituents each independently selected from halo;
wherein each of R₄ and R₅ is independently selected from the group consisting of H, OH, —C₁₋₉ alkyl, —C₁₋₈ haloalkyl, —C₂₋₆ alkenyl, —C₂₋₆ alkynyl, —COR₆ₐ, —C₁₋₄ alkyl-C₃₋₈ cycloalkyl, —C₁₋₄ alkyl-heterocyclyl, —C₃₋₁₀ cycloalkyl, heterocyclyl, —C₆₋₁₀ aryl, 5-10 membered heteroaryl, —C₁₋₄ alkyl-SO₂—C₁₋₄ alkyl, and —C₁₋₄ alkyl-SO₂—C₃₋₈ cycloalkyl;
wherein each of R₄ and R₅ is optionally substituted with one or more substituents each independently selected from the group consisting of H, halogen, oxo, —OH, —CN, —C₁₋₆ alkyl, —C₁₋₆ alkoxy, —C₁₋₆ haloalkoxy, —C₃₋₁₀ cycloalkyl, heterocyclyl, —C₆₋₁₀ aryl, and 5-10 membered heteroaryl; or R₄ and R₅ are combined with atoms to which they are attached to form 3-10 membered heterocyclyl; wherein the 3-10 membered heterocyclyl is optionally substituted with one or more substituents each independently selected from the group consisting of halo, —C₁₋₉ alkyl, and —C₁₋₈ haloalkyl;
wherein R₆ is independently selected from the group consisting of H, C₁₋₉ alkyl, C₁₋₈ haloalkyl, —C₁₋₆ alkyl-alkoxy, —C₁₋₆ alkyl-cycloalkoxy, C₃₋₁₀ cycloalkyl, heterocyclyl, CO₂R₆ₐ, COR₆ₐ, CON(R₆ₐ)(R_{6b});
wherein R₆ is optionally substituted with one or more substituents each independently selected from the group consisting of halogen, —C₁₋₆ alkyl, —C₁₋₆ alkoxy, —C₁₋₆ haloalkoxy, —C₁₋₆ haloalkyl, —C₃₋₁₀ cycloalkyl.

In some embodiments, each of R₄ and R₅ is independently selected from the group consisting of H, —C₁₋₉ alkyl, —C₁₋₈ haloalkyl, —C₁₋₄ alkyl-C₃₋₈ cycloalkyl, —C₁₋₄ alkyl-heterocyclyl, and —C₃₋₁₀ cycloalkyl;

or R₄ and R₅ are combined with atoms to which they are attached to form 3-10 membered heterocyclyl; wherein the 3-10 membered heterocyclyl is optionally substituted with one or more substituents each independently selected from the group consisting of halo, —C₁₋₉ alkyl, and —C₁₋₈ haloalkyl.

In some embodiments, R₆ is independently selected from the group consisting of H, C₁₋₉ alkyl, C₁₋₈ haloalkyl, —C₁₋₆ alkyl-alkoxy, —C₁₋₆ alkyl-cycloalkoxy, C₃₋₁₀ cycloalkyl, heterocyclyl, CO₂R₆ₐ, COR₆ₐ, and CON(R₆ₐ)(R_{6b});
wherein R₆ is optionally substituted with one or more substituents each independently selected from the group consisting of halogen, —C₁₋₆ alkyl, —C₁₋₆ alkoxy, —C₁₋₆ haloalkoxy, —C₁₋₆ haloalkyl, and —C₃₋₁₀ cycloalkyl.

In some embodiments, each of R₆ₐ and R_{6b} is independently selected from the group consisting of H, C₁₋₉ alkyl, C₁₋₈ haloalkyl, C₃₋₁₀ cycloalkyl, and heterocyclyl; or R₆ₐ and R_{6b} are combined with atoms to which they are attached to form a C₃₋₁₀ cycloalkyl or 5-10 membered heterocyclyl; wherein the C₃₋₁₀ cycloalkyl or 5-10 membered heterocyclyl is optionally substituted with one or more substituents each independently selected from the group consisting of halo, —C₁₋₉ alkyl, and —C₁₋₈ haloalkyl;
wherein each of R₆ₐ and R_{6b} is optionally substituted with one or more substituents each independently selected from the group consisting of halogen, —C₁₋₆ alkyl, —C₁₋₆ alkoxy, —C₁₋₆ haloalkoxy, —C₁₋₆ haloalkyl, —C₃₋₁₀ cycloalkyl, and heterocyclyl.

In some embodiments, R₁ is selected from the group consisting of group of

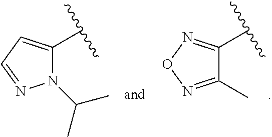

In some embodiments, R₂ and R₃ is independently selected from the group consisting of H, —C₁₋₆ alkyl, C₃₋₁₀ cycloalkyl, and —C₀₋₂ alkyl-C₃₋₁₀ cycloalkyl; or R₂ and R₃ are combined with atoms to which they are attached to form a C₃₋₁₀ cycloalkyl; wherein the C₃₋₁₀ cycloalkyl is optionally substituted with one or more halo.

In some embodiments, the compound of the present application is selected from the group consisting of the following compounds:

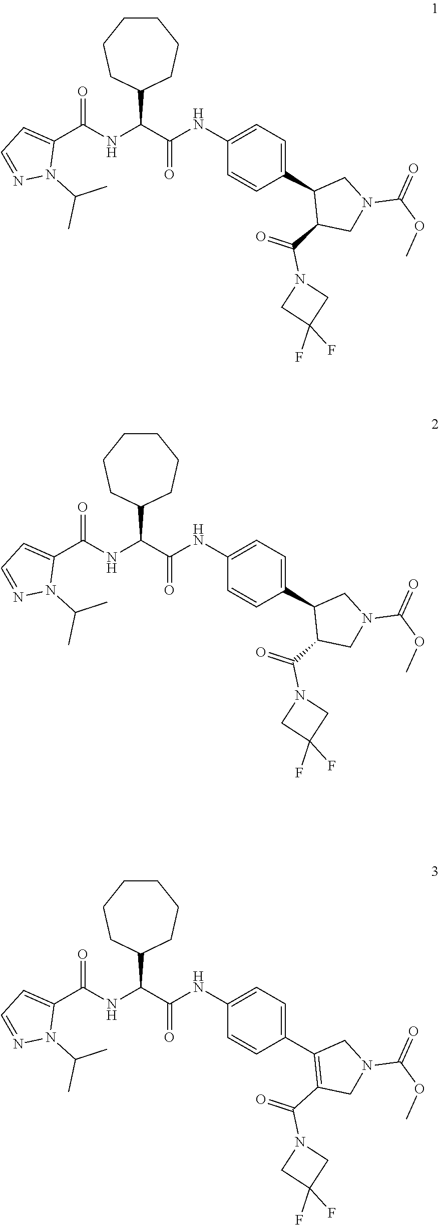 [SMILES: COC(=O)N1C[C@H](c2ccc(NC(=O)[C@@H](NC(=O)c3ccnn3C(C)C)C3CCCCCC3)cc2)[C@H](C(=O)N2CC(F)(F)C2)C1 ; COC(=O)N1C[C@H](C(=O)N2CC(F)(F)C2)[C@@H](c2ccc(NC(=O)[C@@H](NC(=O)c3ccnn3C(C)C)C3CCCCCC3)cc2)C1 ; COC(=O)N1CC(C(=O)N2CC(F)(F)C2)=C(c2ccc(NC(=O)[C@@H](NC(=O)c3ccnn3C(C)C)C3CCCCCC3)cc2)C1]

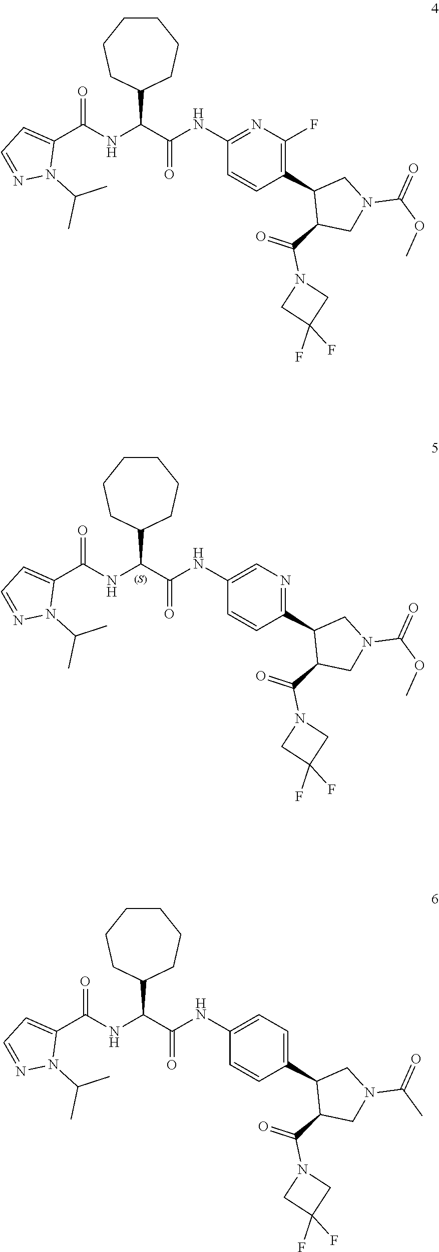

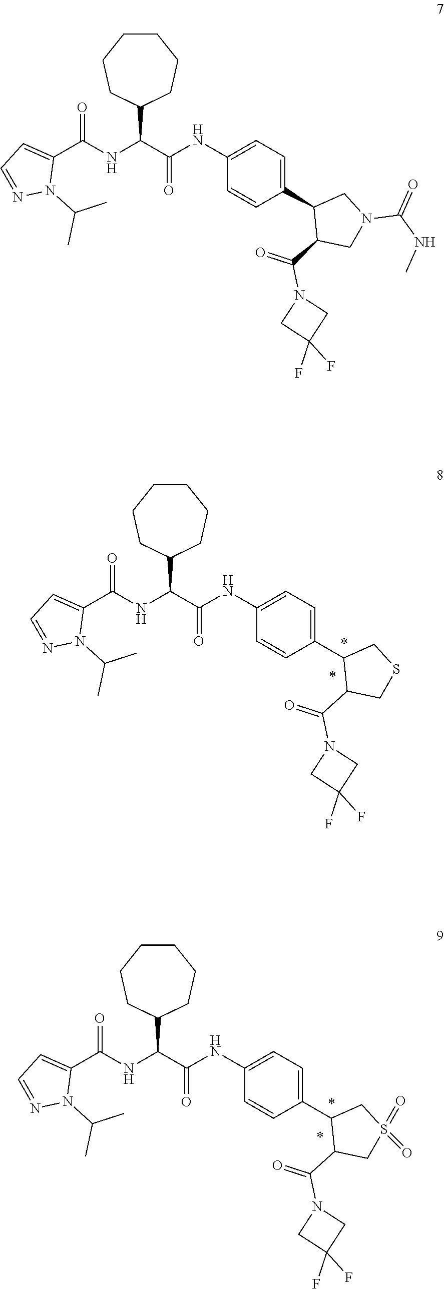 [SMILES: CNC(=O)N1C[C@H](c2ccc(NC(=O)[C@@H](NC(=O)c3ccnn3C(C)C)C3CCCCCC3)cc2)[C@H](C(=O)N2CC(F)(F)C2)C1 ; CC(C)n1nccc1C(=O)N[C@H](C(=O)Nc1ccc(C2CSCC2C(=O)N2CC(F)(F)C2)cc1)C1CCCCCC1 ; CC(C)n1nccc1C(=O)N[C@H](C(=O)Nc1ccc(C2CS(=O)(=O)CC2C(=O)N2CC(F)(F)C2)cc1)C1CCCCCC1]

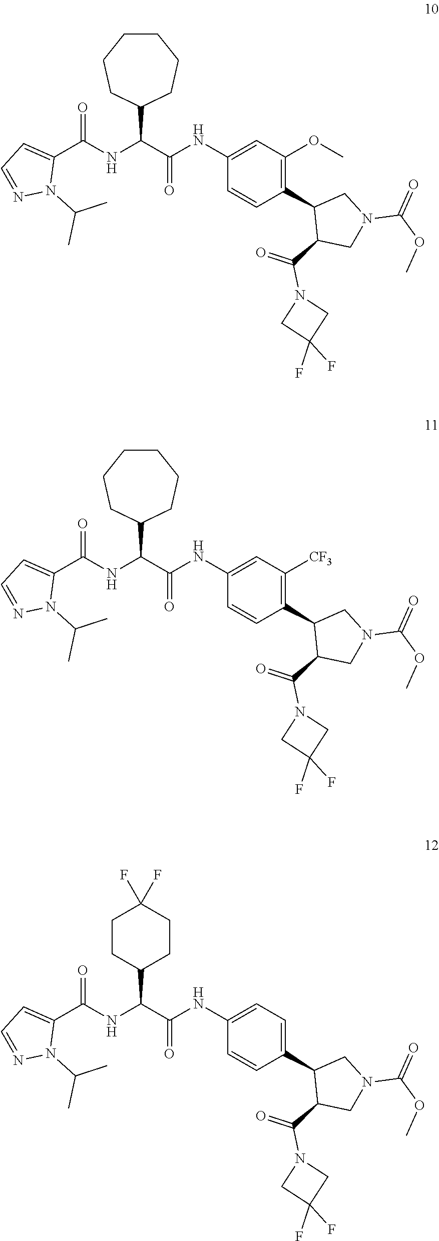 [SMILES: COC(=O)N1C[C@H](c2ccc(NC(=O)[C@@H](NC(=O)c3ccnn3C(C)C)C3CCCCCC3)cc2OC)[C@H](C(=O)N2CC(F)(F)C2)C1 ; COC(=O)N1C[C@H](c2ccc(NC(=O)[C@@H](NC(=O)c3ccnn3C(C)C)C3CCCCCC3)cc2C(F)(F)F)[C@H](C(=O)N2CC(F)(F)C2)C1 ; COC(=O)N1C[C@H](c2ccc(NC(=O)[C@@H](NC(=O)c3ccnn3C(C)C)C3CCC(F)(F)CC3)cc2)[C@H](C(=O)N2CC(F)(F)C2)C1]

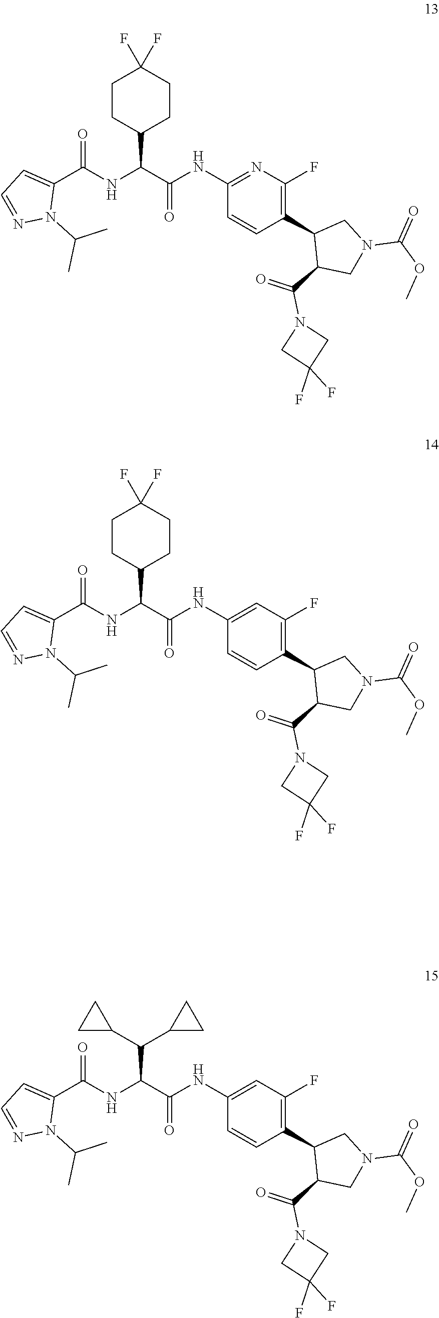 [SMILES: COC(=O)N1C[C@H](c2ccc(NC(=O)[C@@H](NC(=O)c3ccnn3C(C)C)C3CCC(F)(F)CC3)nc2F)[C@H](C(=O)N2CC(F)(F)C2)C1 ; COC(=O)N1C[C@H](c2ccc(NC(=O)[C@@H](NC(=O)c3ccnn3C(C)C)C3CCC(F)(F)CC3)cc2F)[C@H](C(=O)N2CC(F)(F)C2)C1 ; COC(=O)N1C[C@H](c2ccc(NC(=O)[C@@H](NC(=O)c3ccnn3C(C)C)C(C3CC3)C3CC3)cc2F)[C@H](C(=O)N2CC(F)(F)C2)C1]

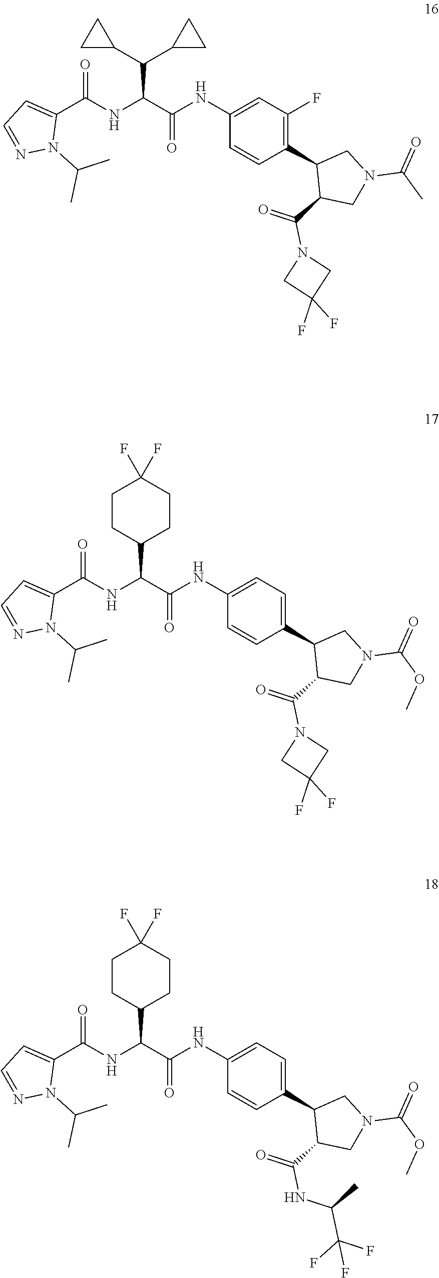 [SMILES: CC(=O)N1C[C@H](c2ccc(NC(=O)[C@@H](NC(=O)c3ccnn3C(C)C)C(C3CC3)C3CC3)cc2F)[C@H](C(=O)N2CC(F)(F)C2)C1 ; COC(=O)N1C[C@H](C(=O)N2CC(F)(F)C2)[C@@H](c2ccc(NC(=O)[C@@H](NC(=O)c3ccnn3C(C)C)C3CCC(F)(F)CC3)cc2)C1 ; COC(=O)N1C[C@H](C(=O)N[C@@H](C)C(F)(F)F)[C@@H](c2ccc(NC(=O)[C@@H](NC(=O)c3ccnn3C(C)C)C3CCC(F)(F)CC3)cc2)C1]

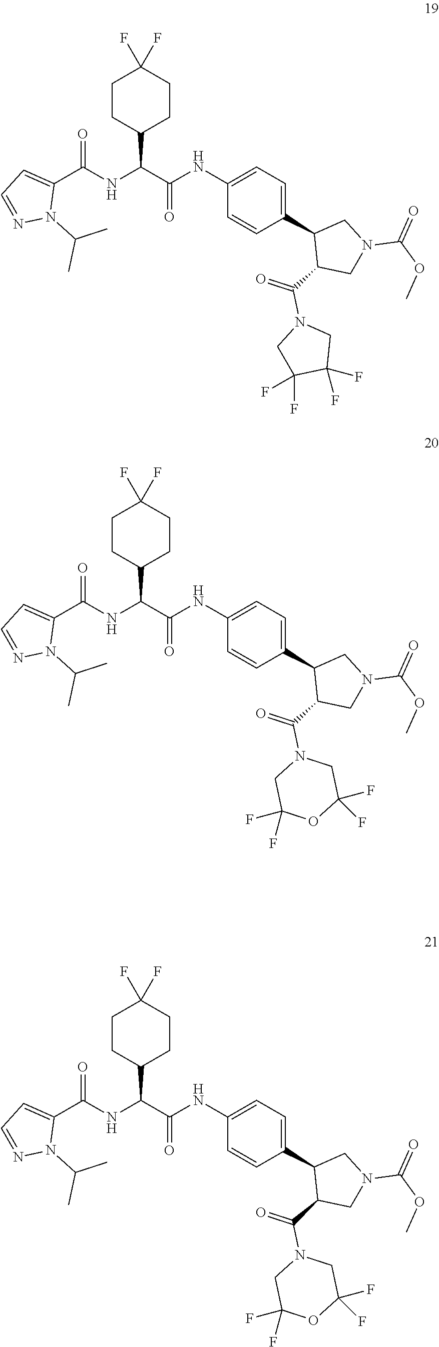 [SMILES: COC(=O)N1C[C@H](C(=O)N2CC(F)(F)C(F)(F)C2)[C@@H](c2ccc(NC(=O)[C@@H](NC(=O)c3ccnn3C(C)C)C3CCC(F)(F)CC3)cc2)C1 ; COC(=O)N1C[C@H](C(=O)N2CC(F)(F)OC(F)(F)C2)[C@@H](c2ccc(NC(=O)[C@@H](NC(=O)c3ccnn3C(C)C)C3CCC(F)(F)CC3)cc2)C1 ; COC(=O)N1C[C@H](c2ccc(NC(=O)[C@@H](NC(=O)c3ccnn3C(C)C)C3CCC(F)(F)CC3)cc2)[C@H](C(=O)N2CC(F)(F)OC(F)(F)C2)C1]

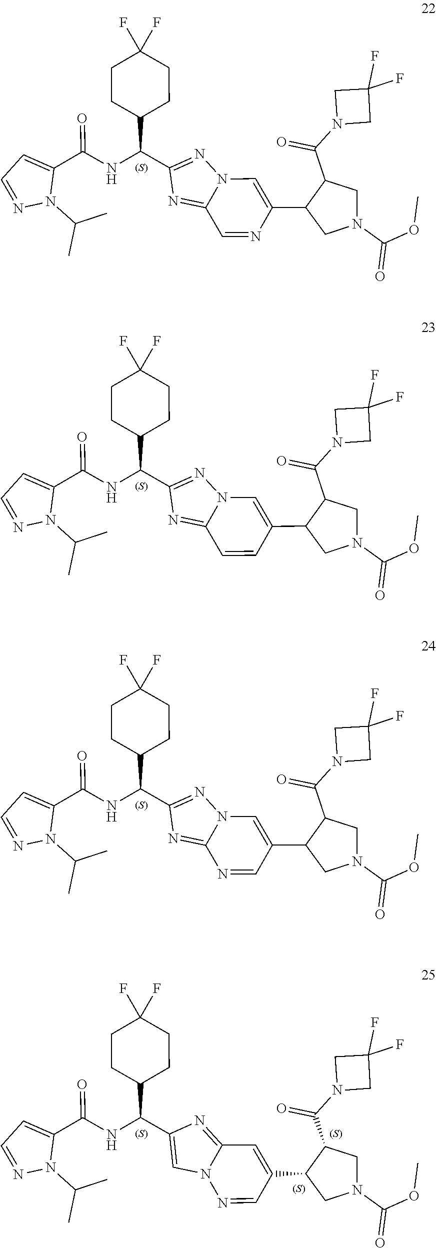

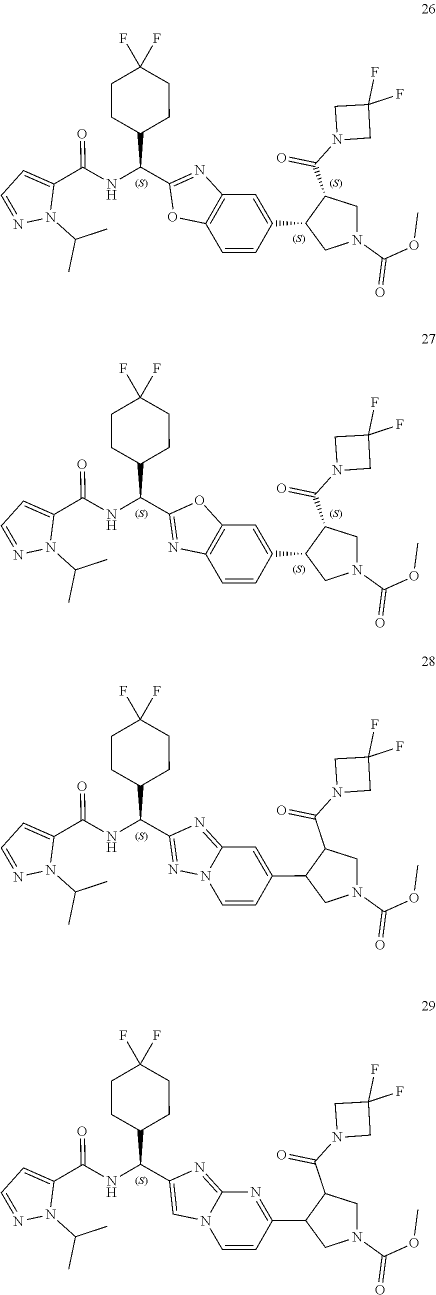

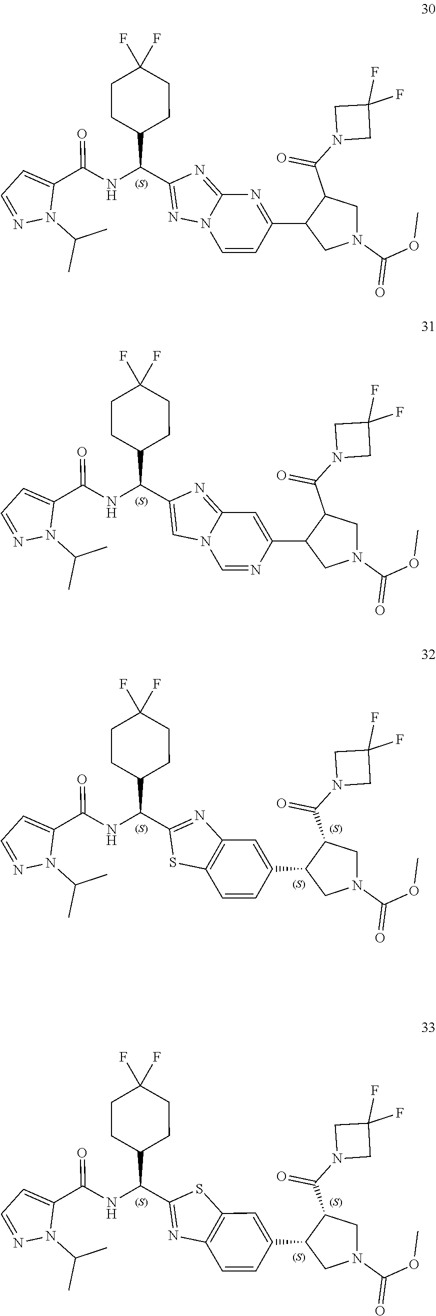

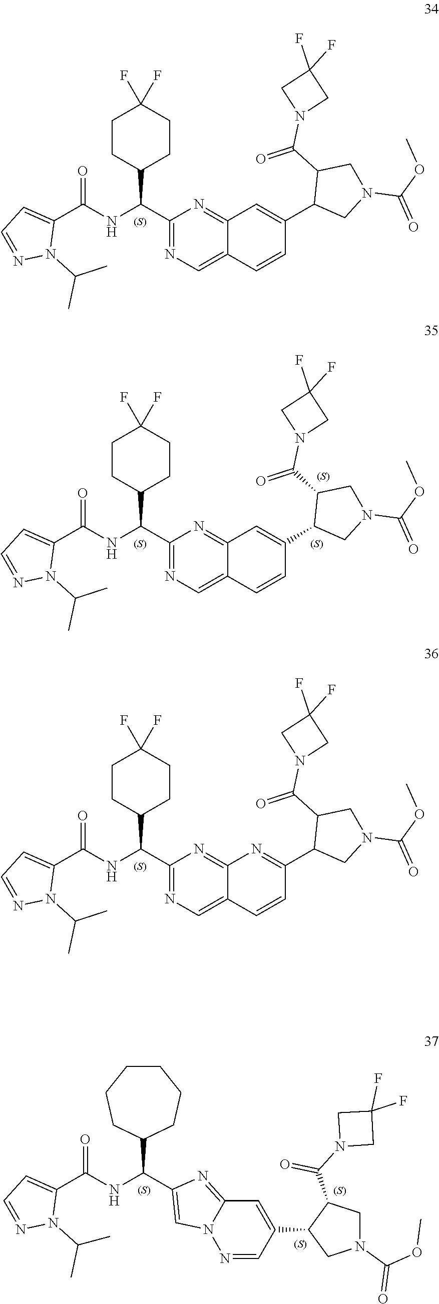

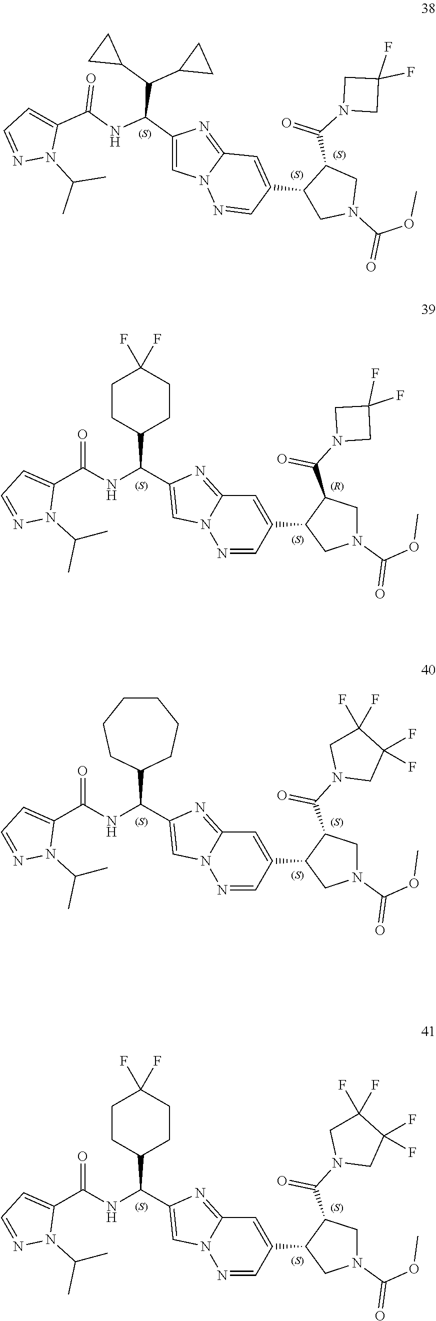

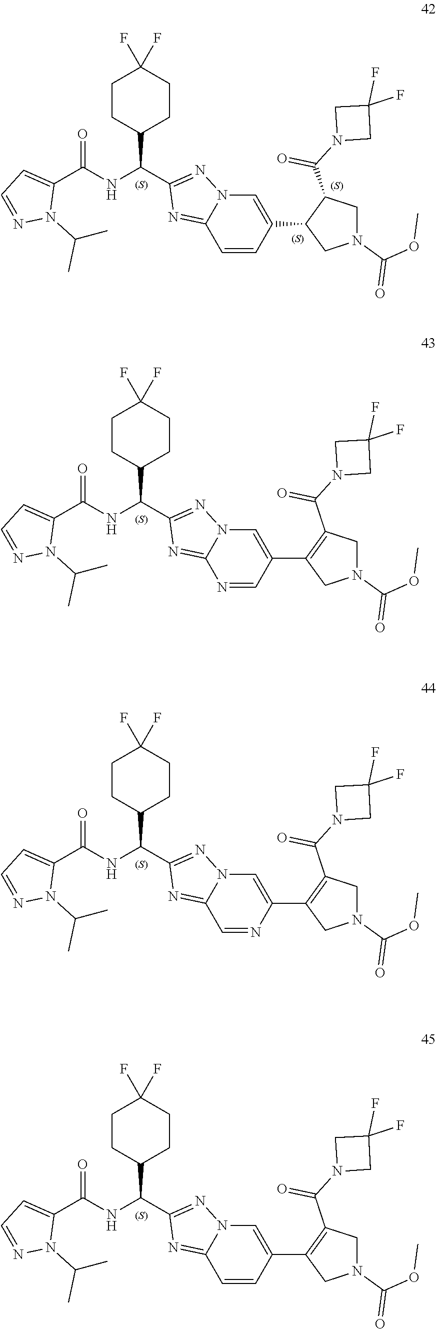

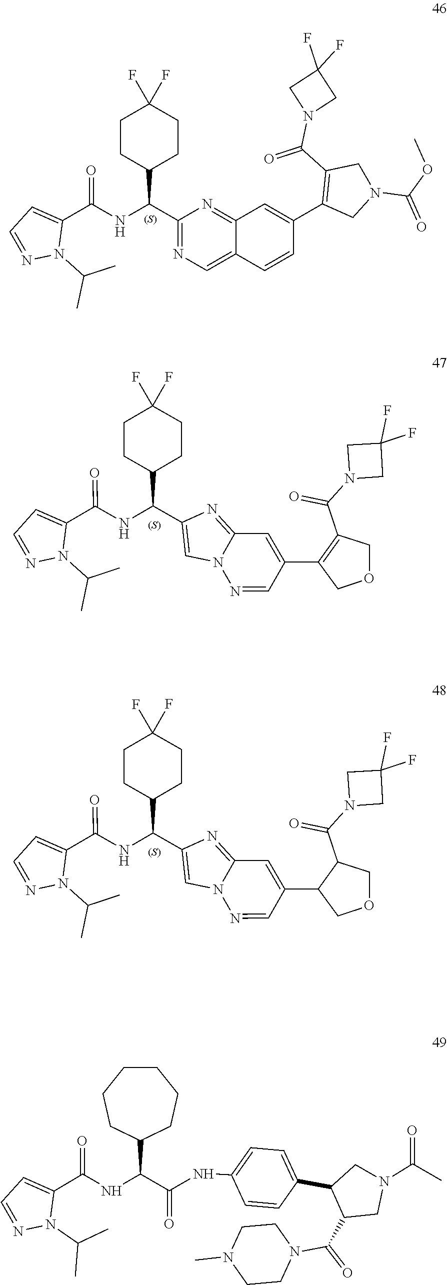

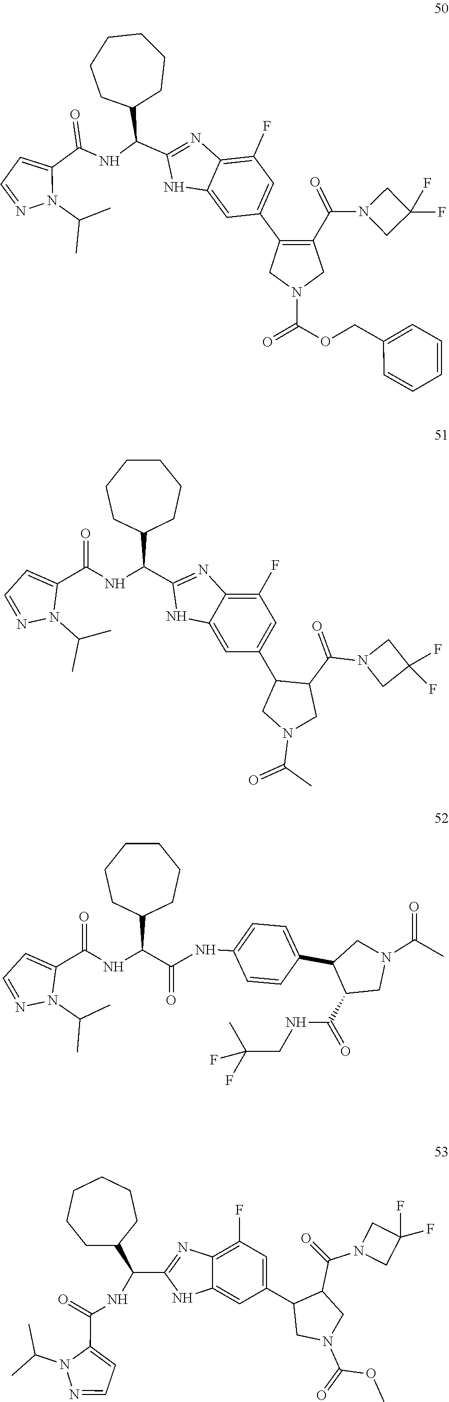

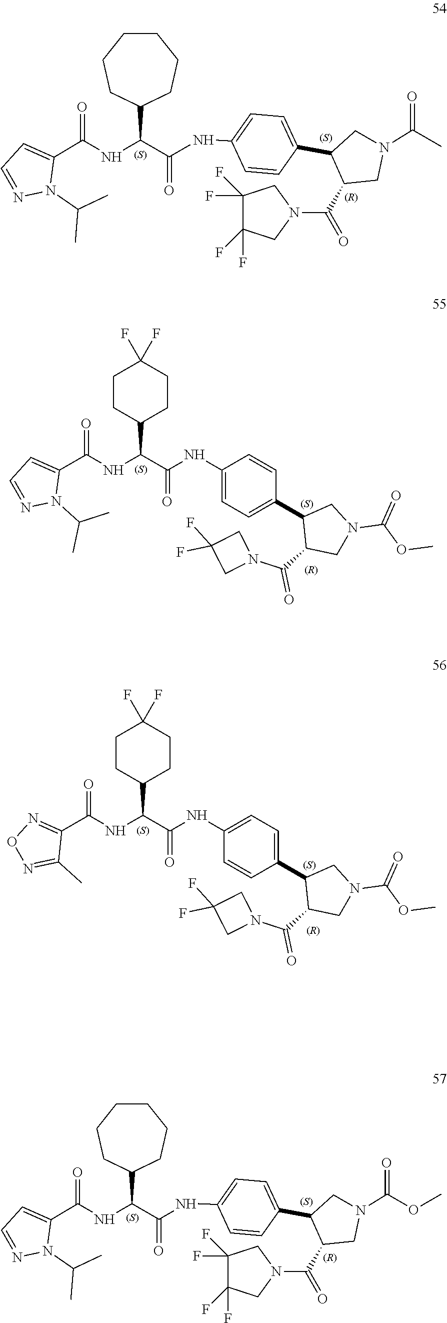

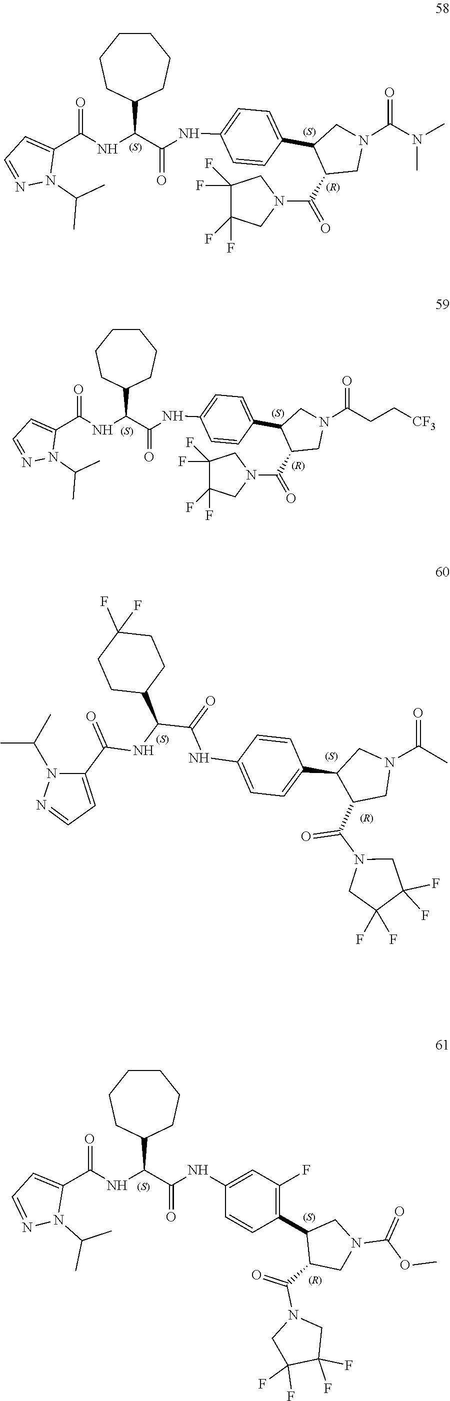

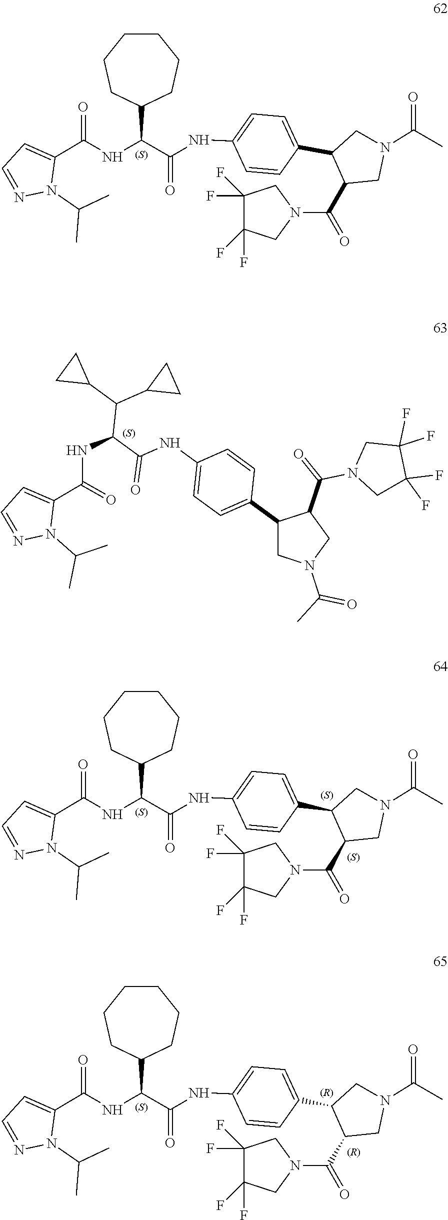

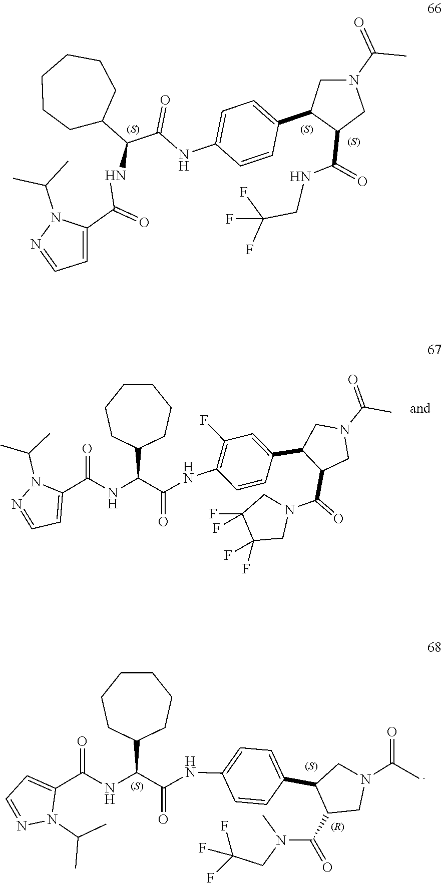

### III. Methods of Treatment

Another aspect of the present application relates to a method for preventing, treating or ameliorating a symptom of an IL-17A mediated inflammatory syndrome, disorder, or disease with the compound of the present application. In some embodiments, the method comprises the step of administering to a subject in need of such treatment an effective amount of a compound of Formula I, Ia, Ib, IIa, IIb, or a pharmaceutically acceptable salt thereof.

### EXAMPLES

### Synthesis

The compounds of the disclosure may be prepared using methods disclosed herein and routine modifications thereof which will be apparent given the disclosure herein and methods well known in the art. Conventional and well-known synthetic methods may be used in addition to the teachings herein. The synthesis of typical compounds of Formula I, or a pharmaceutically acceptable salt thereof, e.g., compounds having structures described by one or more of Formula I, or other formulas or compounds disclosed herein, may be accomplished as described in the following examples.

### General Syntheses

Typical embodiments of compounds in accordance with the present application may be synthesized using the general reaction schemes and/or examples described below. It will be apparent given the description herein that the general schemes may be altered by substitution of the starting materials with other materials having similar structures to result in products that are correspondingly different. Descriptions of syntheses follow to provide numerous examples of how the starting materials may vary to provide corresponding products. Starting materials are typically obtained from commercial sources or synthesized using published methods for synthesizing compounds which are embodiments of the present application, inspection of the structure of the compound to be synthesized will provide the identity of each substituent group The identity of the final product will generally render apparent the identity of the necessary starting materials by a simple process of inspection, given the examples herein. Group labels (e.g., R1, R2) used in the reaction schemes herein are for illustrative purposes only and unless otherwise specified do not necessarily match by name or function the labels used elsewhere to describe compounds of Formula I or aspects or fragments thereof.

### Synthetic Reaction Parameters

The compounds of this disclosure can be prepared from readily available starting materials using, for example, the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts (1999) Protecting Groups in Organic Synthesis, 3rd Edition, Wiley, New York, and references cited therein.

Furthermore, the compounds of the present application may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplemental (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

The terms “solvent,” “inert organic solvent” or “inert solvent” refer to a solvent inert under the conditions of the reaction being described in conjunction therewith (including, for example, benzene, toluene, acetonitrile, tetrahydrofuran (“THF”), N, N-dimethylformamide (“DMF”), chloroform, methylene chloride (or dichloromethane), diethyl ether, methanol, pyridine and the like). Unless specified to the contrary, the solvents used in the reactions of the present application are inert organic solvents, and the reactions are carried out under an inert gas, preferably nitrogen.

The term “q.s.” means adding a quantity sufficient to achieve a stated function, e.g., to bring a solution to the desired volume (i.e., 100%). Compounds as provided herein may be synthesized according to the general schemes provided below. In the Schemes below, it should be appreciated that each of the compounds shown therein may have protecting groups as required present at any step. Standard protecting groups are well within the prevue of one skilled in the art.

### Preparation Example 1: Synthesis of Intermediate

### Int-1 (S,S): methyl (3S,4S)-1-benzyl-4-(4-((tert-butoxycarbonyl)amino)phenyl)pyrrolidine-3-carboxylate and Int-1 (R,R): methyl (3R,4R)-1-benzyl-4-(4-((tert-butoxycarbonyl)amino)phenyl)pyrrolidine-3-carboxylate

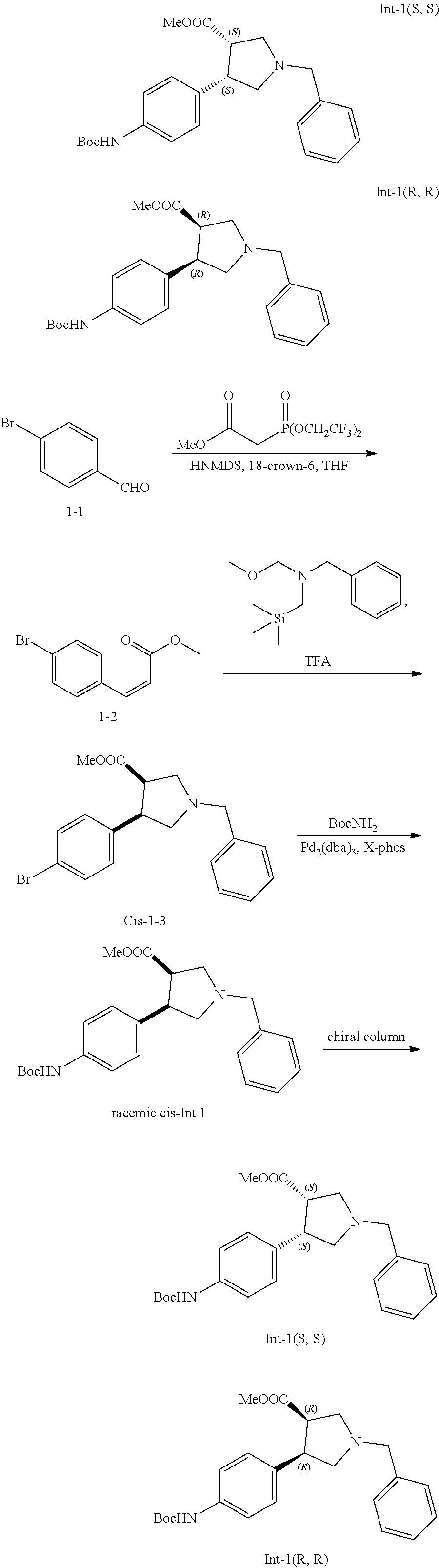

The solution of methyl P,P-bis(2,2,2-trifluoroethyl)phosphonoacetate (1.78 mL, 8.4 mmol) and 18-crown-6 (3.96 g, 15 mmol) in THF (20 mL) under nitrogen was treated with KHMDS (0.5 M in toluene, 16.8 mL, 8.4 mmol) at −78° C. and was stirred for 30 min at this temperature. To this suspension, a solution of compound 1-1 (1.1 g, 6 mmol) in THF (2 mL) was added dropwise, and the reaction mixture was stirred 2 hours at −78° C. The reaction was quenched with sat. NH₄Cl solution and the organic phase was washed with water and brine, dried over MgSO₄ and concentrated in vacuo. Purification by column chromatography on silica gel, Int 1-2 was obtained, 0.7 g, yield: 47.9%. m/z=241.0 [M+H]⁺.

Compound 1-2 (600 mg, 2.49 mmol), N-benzyl-1-methoxy-N-((trimethylsilyl)methyl) methanamine (709 mg, 2.99 mmol) and Trifluoroacetic acid (TFA) (93.6 mg, 0.82 mmol) was dissolved into DCM (10 ml), and stirred 2 days at 20~25° C. The reaction was added into the sat. NaHCO₃ solution (10 ml) and the organic phase was washed with water and brine, dried over MgSO₄ and concentrated in vacuo. Purification by column chromatography on silica gel, Int 1-3 was obtained, 0.82 g, yield: 88.0%. m/z=374.1 [M+H]⁺.

Int Cis-1-3 (800 mg, 2.14 mmol), BocNH₂ (275 mg, 2.35 mmol), Pd2(dba)3 (18.3 mg, 0.2 mmol), X-Phos (19 mg, 0.4 mmol), Cs₂CO₃ (1.04 g, 3.21 mmol) was added into the dioxane under nitrogen, and stirred 3 hours at 90~100° C. After cooling, EtOAc (20 ml) and water (20 ml) was added into the mixture solution and stirred 10 mins. Then the organic phase was separated, and washed with water and brine, dried over MgSO₄ and concentrated in vacuo. Purification by column chromatography on silica gel, Cis-Int 1 was obtained, 0.64 g, yield: 72.9%, m/z=411.2 [M+H]⁺. Cis-Int 1 was purified by chiral column (Welch XT C18 150 mm*21.2 mm, 5 um) to afford Int-1 (S,S): methyl (3S,4S)-1-benzyl-4-(4-((tert-butoxycarbonyl)amino)phenyl)pyrrolidine-3-carboxylate and Int-1 (R,R): methyl (3R,4R)-1-benzyl-4-(4-((tert-butoxycarbonyl)amino)phenyl)pyrrolidine-3-carboxylate

### Int 2 (R,S): methyl (3R,4S)-1-benzyl-4-(4-((tert-butoxycarbonyl)amino)phenyl)pyrrolidine-3-carboxylate and Int 2 (S,R): methyl (3R,4S)-1-benzyl-4-(4-((tert-butoxycarbonyl)amino)phenyl)pyrrolidine-3-carboxylate

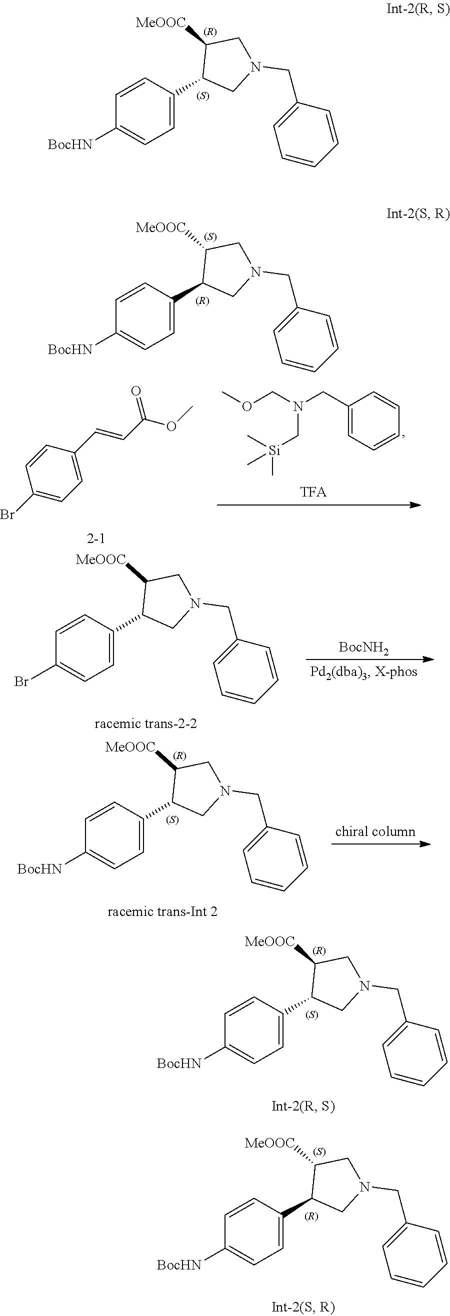

Following the produce of Int-1 (S,S), the racemic trans-Int 2 can be prepared by an E-alkene. After purification by chiral column (Welch XT C18 150 mm*21.2 mm, 5 um) to afford title compound.

### Int 3: methyl (3S,4R)-1-benzyl-4-(5-((tert-butoxycarbonyl)amino)pyridin-2-yl)pyrrolidine-3-carboxylate

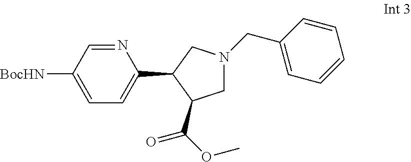 [SMILES: COC(=O)[C@@H]1CN(Cc2ccccc2)C[C@@H]1c1ccc(NC(=O)OC(C)(C)C)cn1]

Following the produce of Int-1 (S,S), the Int 3 was obtained by substituting 5-bromopicolinaldehyde for 1-1.

### Int 4: methyl (3S,4S)-1-benzyl-4-(6-((tert-butoxycarbonyl)amino)-2-fluoropyridin-3-yl)pyrrolidine-3-carboxylate

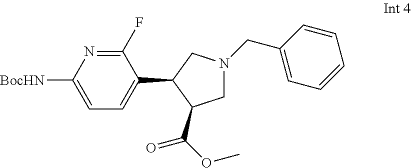 [SMILES: COC(=O)[C@@H]1CN(Cc2ccccc2)C[C@@H]1c1ccc(NC(=O)OC(C)(C)C)nc1F]

Following the produce of Int-1 (S,S), the Int 4 was obtained by substituting 6-bromo-2-fluoronicotinaldehyde for 1-1.

### Int 5: methyl (3S,4S)-1-benzyl-4-(4-((tert-butoxycarbonyl)amino)-2-(trifluoromethyl)phenyl)pyrrolidine-3-carboxylate

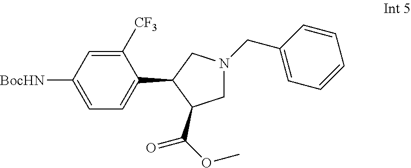 [SMILES: COC(=O)[C@@H]1CN(Cc2ccccc2)C[C@@H]1c1ccc(NC(=O)OC(C)(C)C)cc1C(F)(F)F]

Following the produce of Int-1 (S,S), the Int 5 was obtained by substituting 4-bromo-2-(trifluoromethyl)benzaldehyde for 1-1.

### Int 6: methyl 4-(4-aminophenyl)tetrahydrothiophene-3-carboxylate

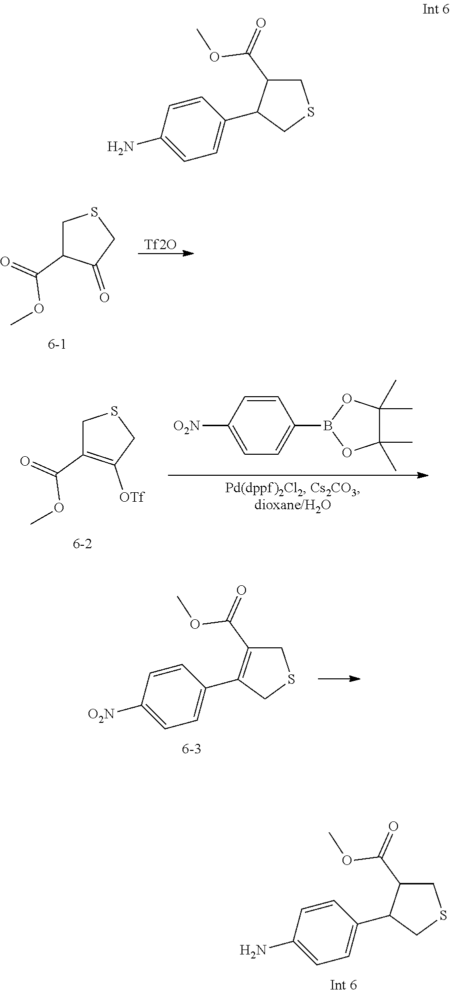

To a solution of 6-1 (1.0 g, 6.24 mmol) and N,N-Diethylethanamine (TEA) (0.95 g, 9.36 mmol) in dichloromethane (DCM) (10 ml) was added dropwise the solution of Tf₂O (1.94 g in DCM, 6.87 mmol) at room temperature and stirred 2 hours. Water (10 ml) was added into the mixture, and stirred 10 mins. The organic layer was separated and washed with water and brine, dried over MgSO₄ and concentrated in vacuo. Purification by column chromatography on silica gel, Int 6-2 was obtained, 1.2 g, yield: 65.6%.

Compound 6-2 (1.0 g, 3.42 mmol), 4,4,5,5-tetramethyl-2-(4-nitrophenyl)-1,3,2-dioxaborolane (0.85 g, 3.42 mmol), Pd(dppf)₂Cl₂ (0.28 g, 0.34 mmol) and Cs₂CO₃ (2.23 g, 6.84 mmol) were added into Dioxane/H₂O (10 ml/2.5 ml) under nitrogen and stirred 2 hours at 90-95° C. After cooling, EtOAc (20 ml) and water (20 ml) was added into the mixture reaction, and stirred 10 mins. The organic layer was separated and washed with water and brine, dried over MgSO₄ and concentrated in vacuo. Purification by column chromatography on silica gel, Int 6-3 was obtained, 0.65 g, yield: 71.6%.

Compound 6-3 (0.65 g, 2.45 mmol) and Pd/C (wt: 10%, 0.065 g) were added into methanol (10 ml) under hydrogen, and stirred overnight at 25~30° C. After filtration, a solution was obtained and concentrated in vacuo to afford compound Int 6, 0.85 g, 95.0%. m/z=238.1 [M+H]⁺.

### Int 7: methyl 4-(4-aminophenyl)tetrahydrothiophene-3-carboxylate 1,1-dioxide

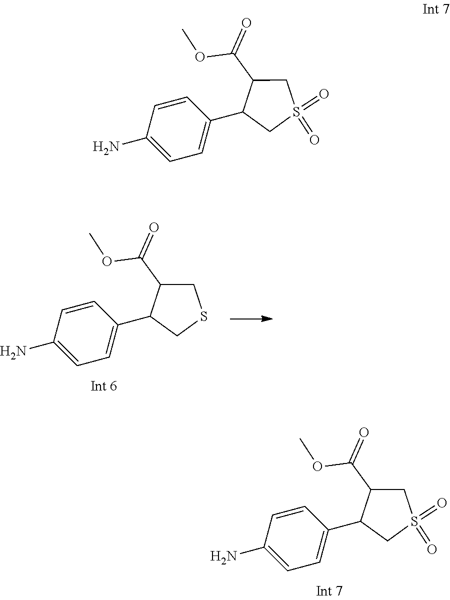 [SMILES: COC(=O)C1CS(=O)(=O)CC1c1ccc(N)cc1 ; COC(=O)C1CSCC1c1ccc(N)cc1 ; COC(=O)C1CS(=O)(=O)CC1c1ccc(N)cc1]

To a solution of Int 6 (0.3 g, 1.26 mmol) in methanol (3 ml) was added H₂O₂ (30%, 1 ml) and Titanium isopropoxide (0.5 ml), and stirred 3 hours at 40-45° C. EtOAc (10 ml) and sat. Na2S2O3 solution (10 ml) was added and stirred 30 mins. The organic phase was separated and washed with water and brine, dried over MgSO₄ and concentrated in vacuo to afford Int 7, 0.25 g, yield: 73.4%. m/z=270.1 [M+H]⁺.

### Int 8: methyl 4-(2-((S)-((tert-butoxycarbonyl)amino)(4,4-difluorocyclohexyl)methyl)-[1,2,4]triazolo[1,5-a]pyrazin-6-yl)pyrrolidine-3-carboxylate

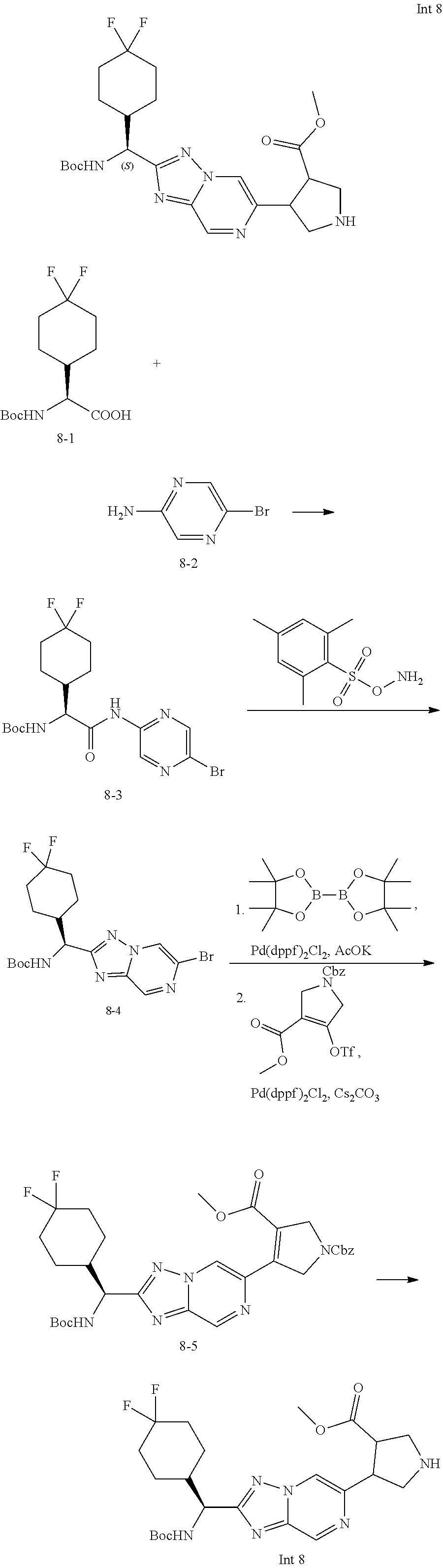

Compound 8-1 (2.0 g, 6.82 mmol), HATU (3.11 g, 8.18 mmol) and TEA (1.0 g, 10.23 mmol) was added into DCM (15 ml), and stirred 1 hours at 20-25° C. Compound 8-2 (1.18 g, 6.82 mmol) was added and stirred 4 hours. Water (15 ml) was added and stirred 15 mins. The organic layers was separated and washed with water and brine, dried over MgSO₄ and concentrated in vacuo to afford 8-3, 1.87 g, yield: 61.0%. m/z=449.1 [M+H]⁺.

To a solution of Compound 8-3 (1.80 g, 4.01 mmol) in DCM (20 ml) was added a solution of O-(mesitylsulfonyl) hydroxylamine (1.29 g, 6.01 mmol) in DCM (5 ml) at 0-5° C. and stirred 1 hour. Warming the mixture reaction to 40~45° C., and keep stirring overnight. Water (20 ml) was added into the mixture reaction and stirred 15 mins. The organic layer was separated and washed with water and brine, dried over MgSO₄ and concentrated in vacuo. Purification by column chromatography on silica gel, Int 8-4 was obtained, 0.55 g, yield: 30.8%. m/z=446.1 [M+H]⁺.

Compound 8-4 (0.55 g, 1.23 mmol), 4,4,4′,4′,5,5,5′,5′-octamethyl-2,2′-bi (1,3,2-dioxaborolane) (0.328 g, 1.29 mmol), Pd(dppf)₂Cl₂ (0.11 g, 0.14 mmol) and AcOK (0.18 g, 1.85 mmol) were added into Dioxane (10 ml) under nitrogen and stirred 2 hours at 80-85° C. After cooling, 1-benzyl 3-methyl 4-(((trifluoromethyl) sulfonyl)oxy)-2,5-dihydro-1H-pyrrole-1,3-dicarboxylate (0.50 g, 1.23 mmol), H₂O (2 ml) and Cs₂CO₃ (0.80 g, 2.46 mmol) was added into the mixture reaction and stirred 3 hours at 90-95° C. After cooling, EtOAc (20 ml) and H₂O (20 ml) was added and stirred 15 mins. The organic layer was separated and washed with water and brine, dried over MgSO₄ and concentrated in vacuo. Purification by column chromatography on silica gel, Int 8-5 was obtained, 0.35 g, yield: 45.3%.

Compound 8-5 (0.35 g, 0.56 mmol) and Pd/C (wt: 10%, 0.035 g) were added into methanol (10 ml) under hydrogen, and stirred overnight at 25~30° C. After filtration, a solution was obtained and concentrated in vacuo to afford compound Int 8, 0.27 g, 96.3%. m/z=495.3 [M+H]⁺.

The triazole derivative Int 9-11 can be prepared followed the procedure of Int 8 using a suitable reagent.

| Intermediate | Structure | m/z [M + H]⁺ |
|---|---|---|
| Int 9 | 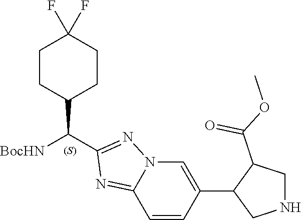 | 494.3 |
| Int 10 | 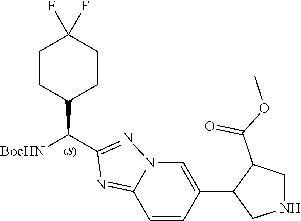 | 495.3 |
| Int 11 | 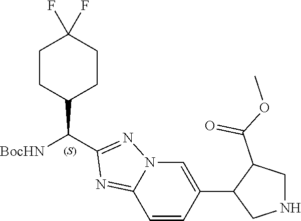 | 495.3 |

### Int 12: tert-butyl (S)-(3-chloro-1-(4,4-difluorocyclohexyl)-2-oxopropyl)carbamate

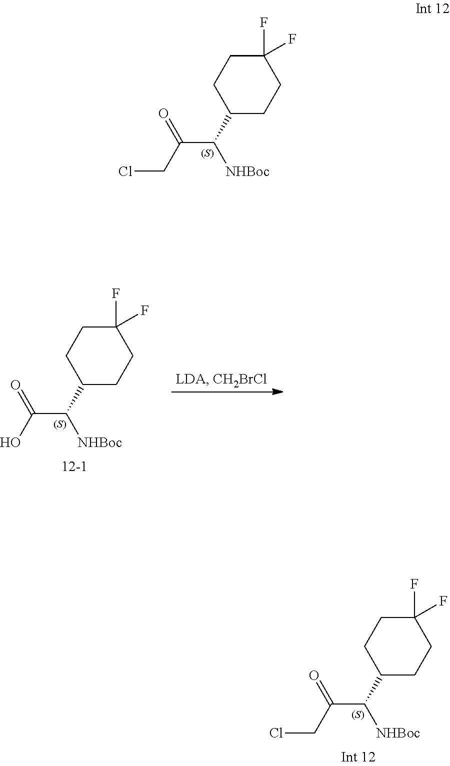

To a solution of 12-1 (5 g, 17.1 mmol) and bromochloromethane (11.0 g, 85.5 mmol) in THF (25 ml) was added LDA (2.0M in THF/Hexane (12:25), 26 ml, 51.3 mmol) and stirred 3 hours at −78° C. The reaction was quenched with sat. NH₄Cl solution and the organic phase was washed with water and brine, dried over MgSO₄ and concentrated in vacuo. Purification by column chromatography on silica gel, Int 12 was obtained, 3.7 g, yield: 66.6%. m/z=326.1 [M+H]⁺.

### Int 13: tert-butyl (S)-((7-bromoimidazo[1,2-b]pyridazin-2-yl)(4,4-difluorocyclohexyl)methyl)carbamate

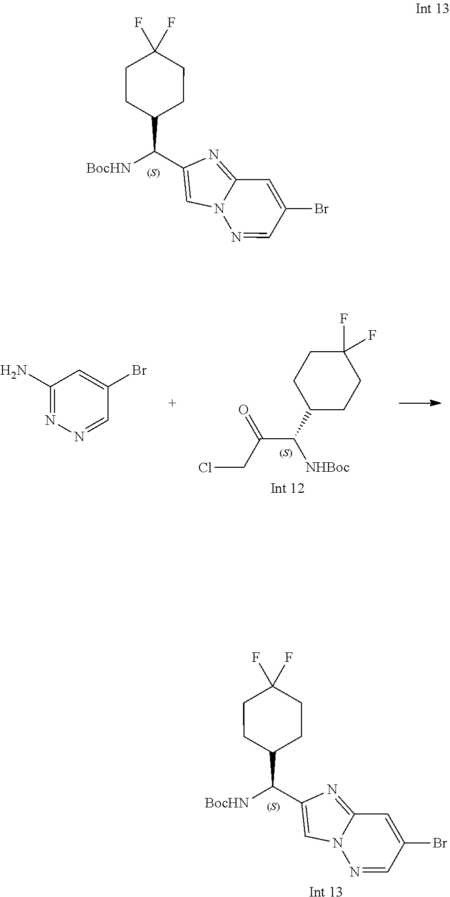

5-bromopyridazin-3-amine (1.0 g, 5.75 mmol), Int 12 (2.81 g, 8.62 mmol) and NaHCO₃ (1.9 g, 23 mmol) was added into the t-BuOH (15 ml) and stirred 48 hours at 130-135° C. in a sealed tube. After cooling, EtOAc (25 ml) and H₂O (25 ml) was added into the mixture and stirred 15 mins. The organic layers was separated and washed with water and brine, dried over MgSO₄ and concentrated in vacuo to afford a residue. The residue was purified column chromatography on silica gel to afford Int 13, 1.32 g, yield: 51.6%. m/z=445.1 [M+H]⁺.

The Imidazole derivative Int 14 and Int 15 can be preparation followed the procedure of Int 13 using a suitable reagent.

### Int 14: tert-butyl (S)-((7-bromoimidazo[1,2-a]pyrimidin-2-yl)(4,4-difluorocyclohexyl)methyl)carbamate

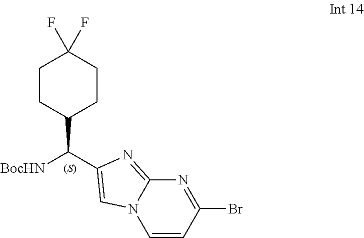

### Int 15: tert-butyl (S)-((7-bromoimidazo[1,2-c]pyrimidin-2-yl)(4,4-difluorocyclohexyl)methyl)carbamate

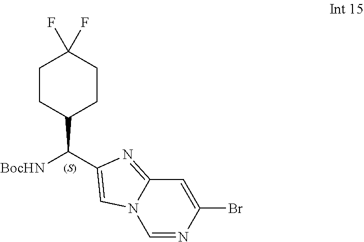

### Int 16: tert-butyl (S)-((7-bromoquinazolin-2-yl)(4,4-difluorocyclohexyl)methyl)carbamate

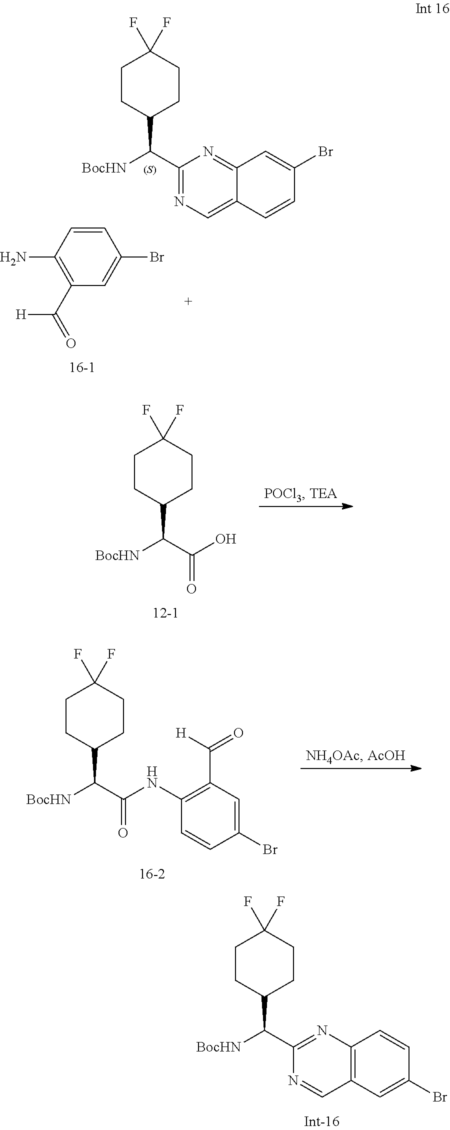

To a solution of 16-1 (1.0 g, 5.0 mmol), TEA (1.01 g, 10 mmol) and 12-1 (1.47 g, 5.0 mmol) in DCM (10 ml) was added dropwise POCl3 (0.77 g, 5 mmol) at 0-5° C. and stirred 2 hours. The reaction was quenched with sat. Na₂CO₃ solution and the organic phase was washed with water and brine, dried over MgSO₄ and concentrated in vacuo. Purification by column chromatography on silica gel, Int 16-2 was obtained, 1.96 g, yield: 82.5%. m/z=475.1 [M+H]⁺.

Compound 16-2 (1.5 g, 3.16 mmol) and NH₄OAc (4.87 g, 63.2 mmol) was added into AcOH (10 ml) and stirred 4 hours at 75-85° C. After cooling, the mixture reaction was quenched with sat. Na₂CO₃ solution and EtOAc (20 ml) was added into the mixture reaction. Then the organic phase was separated and washed with water and brine, dried over MgSO₄ and concentrated in vacuo. Purification by column chromatography on silica gel, Int 16 was obtained, 0.93 g, yield: 64.6%. m/z=456.1 [M+H]⁺.

### Int 17: tert-butyl (S)-((6-bromobenzo[d]oxazol-2-yl)(4,4-difluorocyclohexyl)methyl)carbamate (Int 17)

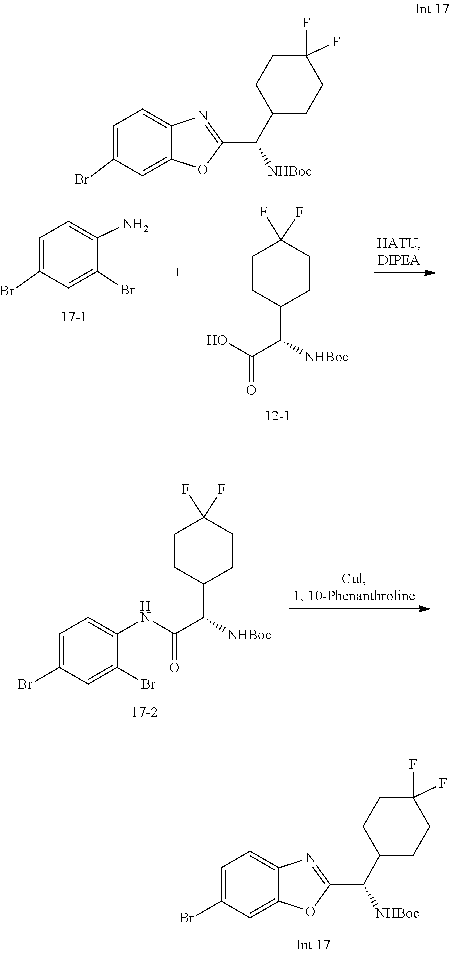

Compound 17-1 (2.0 g, 7.97 mmol), compound 12-1 (2.34 g, 7.97 mmol),HATU (3.63 g, 9.56 mmol), DIPEA (2.06 g, 15.94 mmol) was added into DCM (20 ml), and stirred 3 hours at room temperature. H₂O (20 ml) was added into the mixture and stirred 15 mins. The organic layers was separated and washed with water and brine, dried over MgSO₄ and concentrated in vacuo to afford compound 17-2, 3.86 g, 92.0%. m/z=525.0 [M+H]⁺

Compound 17-2 (2.5 g, 4.75 mmol), CuI (0.9 g, 0.48 mmol), 1,10-phenanthroline (180.5 mg, 0.96 mmol) and Cs₂CO₃ (2.32 g, 7.13 mmol) was added into 1,2-Dimethoxyethane (30 ml) under a nitrogen atmosphere, and reflux the reaction for 24 hours. After cooling, H₂O (30 ml) and DCM (30 ml) was added into the mixture and stirred 15 mins. The organic layers was separated and washed with water and brine, dried over MgSO₄ and concentrated in vacuo a residue. The residue was purified column chromatography on silica gel to afford Int 17, 0.50 g, yield: 23.6%. m/z=445.1 [M+H]⁺.

### Int 18: tert-butyl (S)-((6-bromobenzo[d]thiazol-2-yl)(4,4-difluorocyclohexyl)methyl)carbamate (Int 18)

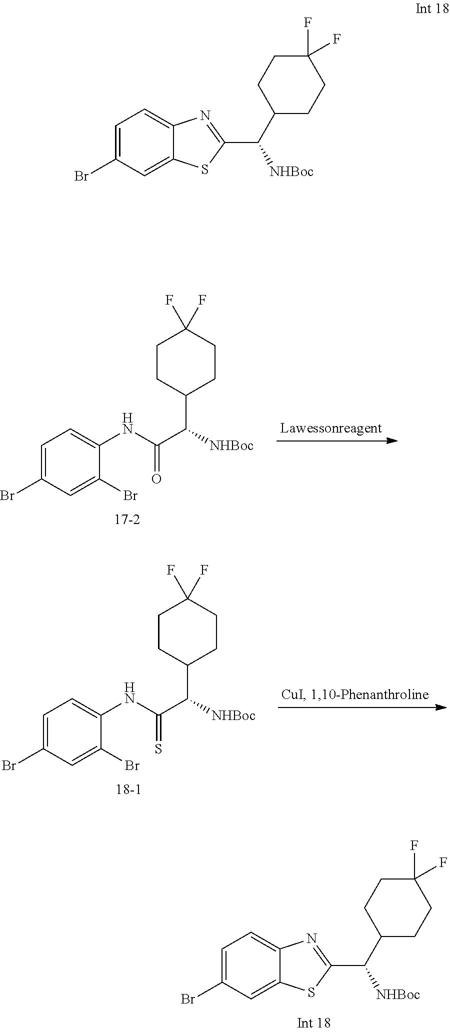 [SMILES: CC(C)(C)OC(=O)N[C@H](c1nc2ccc(Br)cc2s1)C1CCC(F)(F)CC1 ; CC(C)(C)OC(=O)N[C@H](C(=O)Nc1ccc(Br)cc1Br)C1CCC(F)(F)CC1 ; CC(C)(C)OC(=O)N[C@H](C(=S)Nc1ccc(Br)cc1Br)C1CCC(F)(F)CC1 ; CC(C)(C)OC(=O)N[C@H](c1nc2ccc(Br)cc2s1)C1CCC(F)(F)CC1]

Compound 17-2 (0.8 g, 1.52 mmol) and Lawesson's Reagent (0.60 g, 1.52 mmol) was added into toluene (10 ml) and reflux the reaction 4 hours. After cooling, H₂O (10 ml) and EtOAc (10 ml) was added and stirred 10 mins. The organic layers was separated and washed with water and brine, dried over MgSO₄ and concentrated in vacuo a residue. The residue was purified column chromatography on silica gel to afford Int 18-1, 0.21 g, yield: 25.5%. m/z=541.0 [M+H]⁺.

The subsequent reactions refer to the preparation of Int 17 to complete the preparation of Int 18.

The Int 19 and Int 20 can be prepared followed the procedure of Int 17 and Int 18 using a suitable reagent.

### Int 19: tert-butyl (S)-((5-bromobenzo[d]oxazol-2-yl)(4,4-difluorocyclohexyl)methyl)carbamate (Int 19) and Int 20: tert-butyl (S)-((5-bromobenzo[d]thiazol-2-yl)(4,4-difluorocyclohexyl)methyl)carbamate (Int 20)

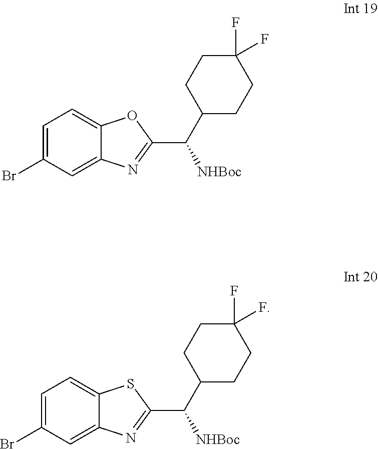 [SMILES: CC(C)(C)OC(=O)N[C@H](c1nc2cc(Br)ccc2o1)C1CCC(F)(F)CC1 ; CC(C)(C)OC(=O)N[C@H](c1nc2cc(Br)ccc2s1)C1CCC(F)(F)CC1]

### Preparation Example 2: Synthesis of Compound 1

### Compound 1: methyl (3S,4S)-3-(4-((S)-2-cycloheptyl-2-(1-isopropyl-1H-pyrazole-5-carboxamido)acetamido)phenyl)-4-(3,3-difluoroazetidine-1-carbonyl)pyrrolidine-1-carboxylate

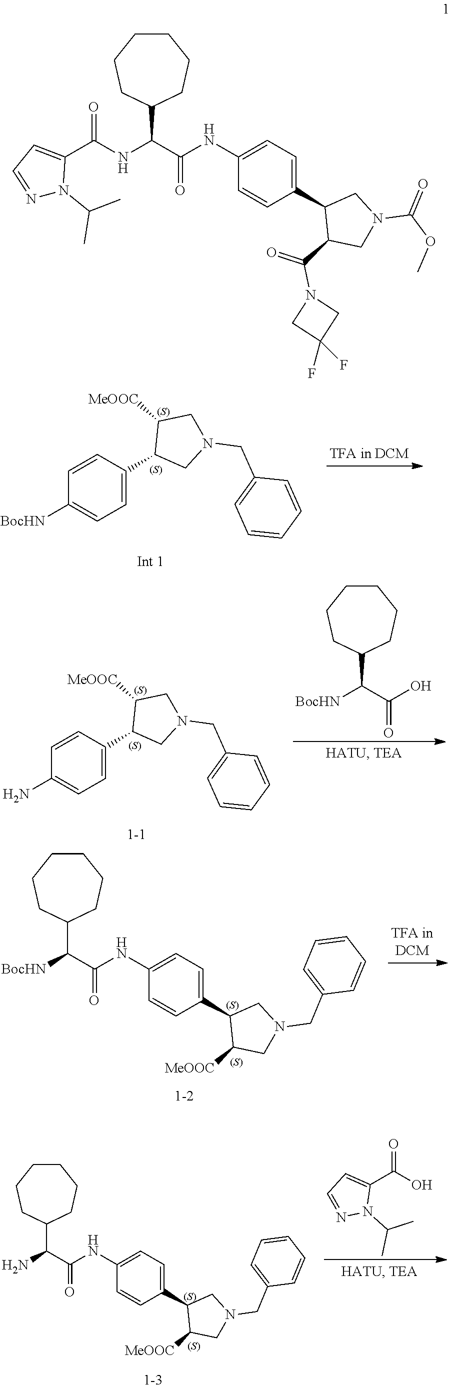

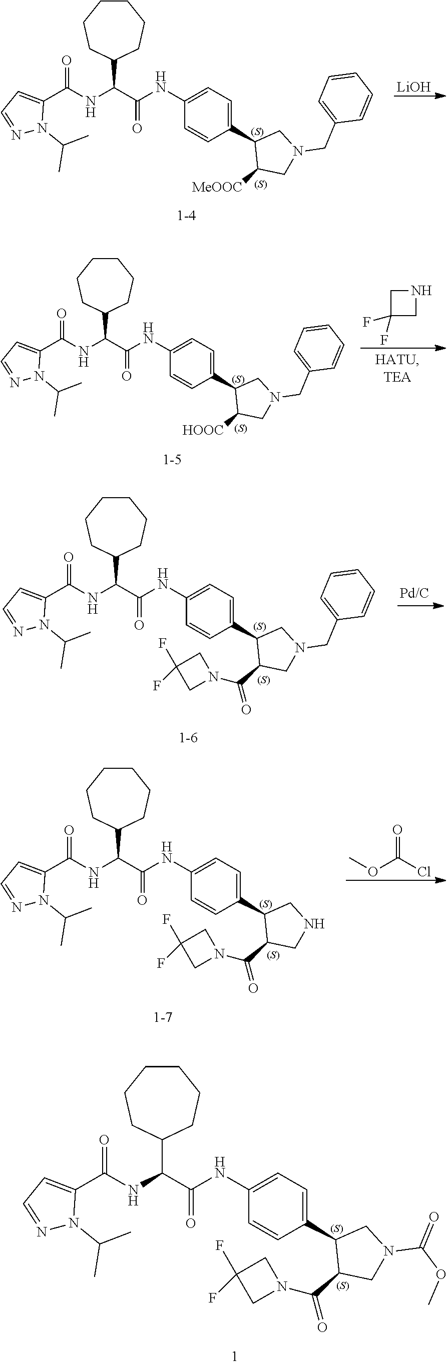

The solution of Int 1 (1.2 g, 2.92 mmol) in DCM (15 ml) was added TFA (5 ml) and stirred 2 hours at room temperature. The mixture solution was concentrated in vacuo to get an oil. The oil was dissolved into DCM (15 ml), followed addition of the sat. NaHCO₃ solution (15 ml). The organic phase was separated and washed with water and brine, dried over MgSO₄ and concentrated in vacuo to afford compound 1-1, 0.83 g, yield: 91.5%. m/z=311.2 [M+H]⁺.

The solution of (S)-2-((tert-butoxycarbonyl)amino)-2-cycloheptylacetic acid (0.77 g, 2.84 mmol) in DCM (20 ml) was added HATU (1.47 g, 3.87 mmol) and TEA (0.52 g, 5.16 mmol) and stirred 30 mins at room temperature. Compound 1-1 (0.80 g, 2.58 mmol) was added into the mixture reaction and keep stirring 2 hours. Water (20 ml) was added and stirred 10 mins. The organic phase was separated and washed with water and brine, dried over MgSO₄ and concentrated in vacuo to afford compound 1-2, 1.03 g, yield: 70.9%. m/z=564.3 [M+H]⁺.

The solution of Int 1-2 (1.0 g, 1.77 mmol) in DCM (10 ml) was added TFA (3 ml) and stirred 2 hours at room temperature. The mixture solution was concentrated in vacuo to get an oil. The oil was dissolved into DCM (10 ml), followed addition of the sat. NaHCO₃ solution (10 ml). The organic phase was separated and washed with water and brine, dried over MgSO₄ and concentrated in vacuo to afford compound 1-3, 0.78 g, yield: 94.8%. m/z=464.3 [M+H]⁺.

The solution of 1-isopropyl-1H-pyrazole-5-carboxylic acid (0.13 g, 0.83 mmol) in DCM (10 ml) was added HATU (0.32 g, 0.83 mmol) and TEA (0.15 g, 1.5 mmol) and stirred 30 mins at room temperature. Compound 1-1 (0.35 g, 0.75 mmol) was added into the mixture reaction and keep stirring 2 hours. Water (10 ml) was added and stirred 10 mins. The organic phase was separated and washed with water and brine, dried over MgSO₄ and concentrated in vacuo to afford compound 1-4, 0.37 g, yield: 81.7%. m/z=564.3 [M+H]⁺.

The solution of 1-4 (0.37 g, 0.62 mmol) in MeOH/H₂O (4 ml/1 ml) was added LiOH (59 mg, 2.47 mmol) and stirred 1 hour at room temperature. Citric acid solution (2M, 10 ml) was added into the mixture solution, followed the EtOAc (20 ml), and stirred 10 mins. The organic phase was separated and washed with water and brine, dried over MgSO₄ and concentrated in vacuo to afford compound 1-5, 0.31 g, yield: 85.8%. m/z=586.3 [M+H]⁺.

The solution of (S)-2-((tert-butoxycarbonyl)amino)-2-cycloheptylacetic acid (0.1 g, 0.17 mmol) in DCM (5 ml) was added HATU (71 mg, 0.19 mmol) and TEA (34 mg, 0.34 mmol) and stirred 30 mins at room temperature. 3,3-difluoroazetidine (19.1 mg, 0.2 mmol) was added into the mixture reaction and keep stirring 2 hours. Water (50 ml) was added and stirred 10 mins. The organic phase was separated and washed with water and brine, dried over MgSO₄ and concentrated in vacuo to afford compound 1-6, 75 mg, yield: 66.5%. m/z=661.4 [M+H]⁺.

The solution of 1-6 (75 mg, 0.11 mmol) in MeOH was added Pd/C (wt: 10%, 10 mg) and stirred 4 hours under a hydrogen atmosphere at room temperature. After filtration, the solution was concentrated in vacuo to afford compound 1-7, without purification to next step reaction.

The solution of 1-7 in DCM (5 ml) was added dropwise the solution of methyl carbonochloridate (12.7 mg) in DCM (5 ml) at 0-5° C. and stirred 1 hours. Water (10 ml) was added and stirred 10 mins. The organic phase was separated and washed with water and brine, dried over MgSO₄ and concentrated in vacuo to an oil. The oil was purified by preparative liquid phase chromatography (ACN: 0.5% TFA, SunFire@Prep C18 OBD™, 5 uM×19 mm×150 mm), the compound 1 was obtained, 41 mg, yield: 58.2%. LCMS: [M+H]⁺: 629.3.

The following compounds were prepared according to the procedures described herein using the appropriate starting material(s) and intermediate(s) and appropriate protecting group chemistry as needed, and certified by ¹HNMR.

TABLE 1
The compound list

| | | m/z | |
|---|---|---|---|
| No. | Structure | [M + H]⁺ | ¹HNMR |
| 1 | 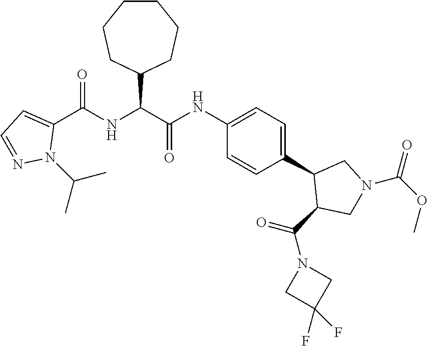 [SMILES: COC(=O)N1C[C@H](c2ccc(NC(=O)[C@@H](NC(=O)c3ccnn3C(C)C)C3CCCCCC3)cc2)[C@H](C(=O)N2CC(F)(F)C2)C1] | 629.3 | ¹H NMR (400 MHz, Chloroform-d) δ 8.18 (s, 1H), 7.50 (dd, J = 8.9, 2.1 Hz, 3H), 7.19 (d, J = 8.3 Hz, 2H), 6.77 (d, J = 8.6 Hz, 1H), 6.56 (d, J = 2.1 Hz, 1H), 5.46-5.33 (m, 1H), 4.57-4.48 (m, 1H), 4.26-4.13 (m, 3H), 4.00-3.74 (m, 2H), 3.71 (s, 3H), 3.69-3.44 (m, 4H), 2.92-2.83 (m, 1H), 2.22-2.08 (m, 1H), 1.88-1.79 (m, 2H), 1.70 (s, 2H), 1.63- 1.55 (m, 2H), 1.54- 1.41 (m, 12H). |
| 2 | 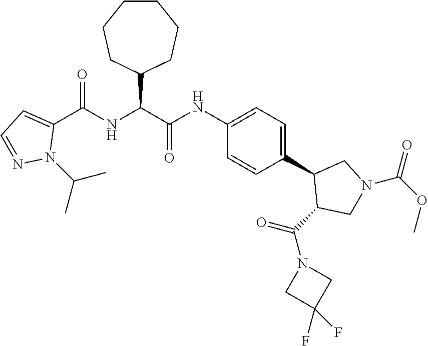 [SMILES: COC(=O)N1C[C@H](C(=O)N2CC(F)(F)C2)[C@@H](c2ccc(NC(=O)[C@@H](NC(=O)c3ccnn3C(C)C)C3CCCCCC3)cc2)C1] | 629.3 | ¹H NMR (400 MHz, Chloroform-d) δ 8.18 (s, 1H), 7.50 (dd, J = 8.9, 2.1 Hz, 3H), 7.19 (d, J = 8.3 Hz, 2H), 6.77 (d, J = 8.6 Hz, 1H), 6.56 (d, J = 2.1 Hz, 1H), 5.46-5.33 (m, 1H), 4.57-4.48 (m, 1H), 4.26-4.13 (m, 3H), 4.00-3.74 (m, 2H), 3.71 (s, 3H), 3.64-3.44 (m, 4H), 2.90-2.83 (m, 1H), 2.21-2.08 (m, 1H), 1.88-1.79 (m, 2H), 1.70 (s, 2H), 1.63- 1.55 (m, 2H), 1.54- 1.41 (m, 12H). |
| 3 | 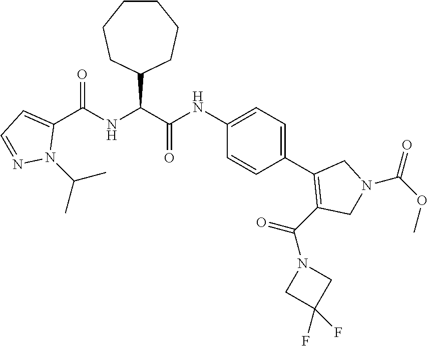 [SMILES: COC(=O)N1CC(C(=O)N2CC(F)(F)C2)=C(c2ccc(NC(=O)[C@@H](NC(=O)c3ccnn3C(C)C)C3CCCCCC3)cc2)C1] | 627.3 | ¹H NMR (400 MHz, Chloroform-d) δ 8.19 (s, 1H), 7.50 (dd, J = 8.9, 2.1 Hz, 3H), 7.19 (d, J = 8.3 Hz, 2H), 6.77 (d, J = 8.6 Hz, 1H), 6.56 (d, J = 2.1 Hz, 1H), 5.46-5.33 (m, 1H), 4.57-4.48 (m, 1H), 4.26-4.13 (m, 3H), 4.00-3.74 (m, 2H), 3.73 (s, 3H), 3.64-3.44 (m, 4H), 1.88-1.79 (m, 2H), 1.70 (s, 2H), 1.63- 1.55 (m, 2H), 1.54- 1.41 (m, 12H). |
| 4 | 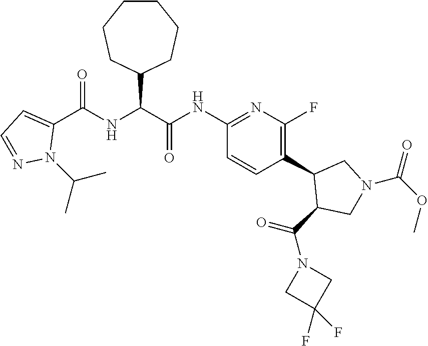 [SMILES: COC(=O)N1C[C@H](c2ccc(NC(=O)[C@@H](NC(=O)c3ccnn3C(C)C)C3CCCCCC3)nc2F)[C@H](C(=O)N2CC(F)(F)C2)C1] | 648.3 | ¹H NMR (400 MHz, Chloroform-d) δ 8.20 (s, 1H), 7.50 (dd, J = 8.9, 2.1 Hz, 2H), 7.19 (d, J = 8.3 Hz, 1H), 6.82 (d, J = 8.6 Hz, 1H), 6.56 (d, J = 2.1 Hz, 1H), 5.46-5.33 (m, 1H), 4.57-4.48 (m, 1H), 4.26-4.13 (m, 3H), 4.00-3.74 (m, 2H), 3.71 (s, 3H), 3.64-3.44 (m, 4H), 2.90-2.83 (m, 1H), 2.21-2.08 (m, 1H), 1.88-1.79 (m, 2H), 1.70 (s, 2H), 1.63- 1.55 (m, 2H), 1.54- 1.41 (m, 12H). |
| 5 | 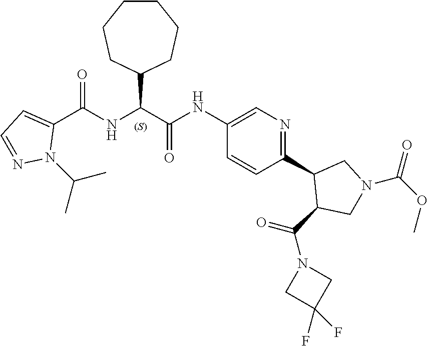 | 630.3 | ¹H NMR (400 MHz, Chloroform-d) δ 8.22 (s, 1H), 7.50 (dd, J = 8.9, 2.1 Hz, 2H), 7.19 (d, J = 8.3 Hz, 2H), 6.82 (d, J = 8.6 Hz, 1H), 6.56 (d, J = 2.1 Hz, 1H), 5.46-5.33 (m, 1H), 4.57-4.48 (m, 1H), 4.26-4.13 (m, 3H), 4.00-3.74 (m, 2H), 3.71 (s, 3H), 3.64-3.44 (m, 4H), 2.90-2.83 (m, 1H), 2.21-2.08 (m, 1H), 1.88-1.79 (m, 2H), 1.70 (s, 2H), 1.63- 1.55 (m, 2H), 1.54- 1.41 (m, 12H). |
| 6 | 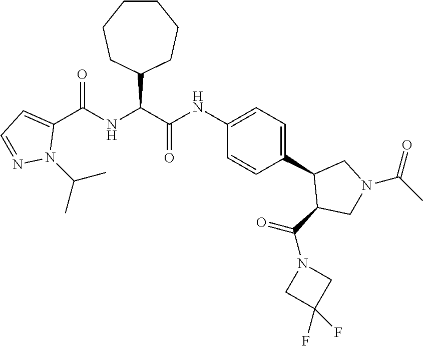 [SMILES: CC(=O)N1C[C@H](c2ccc(NC(=O)[C@@H](NC(=O)c3ccnn3C(C)C)C3CCCCCC3)cc2)[C@H](C(=O)N2CC(F)(F)C2)C1] | 613.3 | ¹H NMR (400 MHz, Chloroform-d) δ 8.18 (s, 1H), 7.50 (dd, J = 8.9, 2.1 Hz, 3H), 7.19 (d, J = 8.3 Hz, 2H), 6.77 (d, J = 8.6 Hz, 1H), 6.56 (d, J = 2.1 Hz, 1H), 5.46-5.33 (m, 1H), 4.57-4.48 (m, 1H), 4.26-4.13 (m, 3H), 4.00-3.74 (m, 2H), 3.69-3.44 (m, 4H), 2.92-2.83 (m, 1H), 2.22-2.12 (m, 1H), 2.10 (s, 3H), 1.88-1.79 (m, 2H), 1.70 (s, 2H), 1.63- 1.55 (m, 2H), 1.54- 1.41 (m, 12H). |
| 7 | 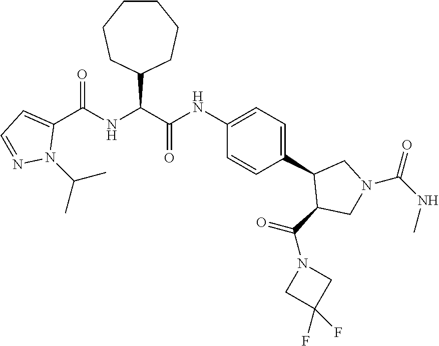 [SMILES: CNC(=O)N1C[C@H](c2ccc(NC(=O)[C@@H](NC(=O)c3ccnn3C(C)C)C3CCCCCC3)cc2)[C@H](C(=O)N2CC(F)(F)C2)C1] | 628.3 | ¹H NMR (400 MHz, Chloroform-d) δ 8.18 (s, 1H), 7.87 (s, 1H), 7.50 (dd, J = 8.9, 2.1 Hz, 3H), 7.19 (d, J = 8.3 Hz, 2H), 6.77 (d, J = 8.6 Hz, 1H), 6.56 (d, J = 2.1 Hz, 1H), 5.46-5.33 (m, 1H), 4.57-4.48 (m, 1H), 4.26-4.13 (m, 3H), 4.00-3.74 (m, 2H), 3.69-3.44 (m, 4H), 2.92-2.83 (m, 1H), 2.70 (s, 3H), 2.22- 2.12 (m, 1H), 1.88- 1.79 (m, 2H), 1.70 (s, 2H), 1.63-1.55 (m, 2H), 1.54-1.41 (m, 12H). |
| 8 | 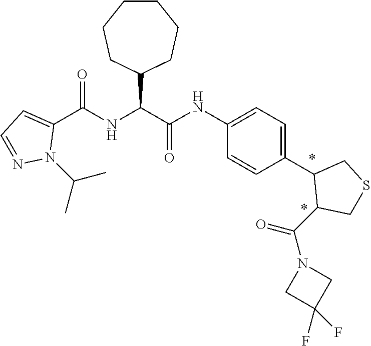 [SMILES: CC(C)n1nccc1C(=O)N[C@H](C(=O)Nc1ccc(C2CSCC2C(=O)N2CC(F)(F)C2)cc1)C1CCCCCC1] | 588.3 | ¹H NMR (400 MHz, Chloroform-d) δ 8.18 (s, 1H), 7.50 (dd, J = 8.9, 2.1 Hz, 3H), 7.19 (d, J = 8.3 Hz, 2H), 6.77 (d, J = 8.6 Hz, 1H), 6.56 (d, J = 2.1 Hz, 1H), 5.46-5.33 (m, 1H), 4.57-4.48 (m, 1H), 4.26-4.13 (m, 3H), 4.00-3.74 (m, 2H), 3.69-3.44 (m, 4H), 2.92-2.83 (m, 1H), 2.22-2.12 (m, 1H), 1.88-1.79 (m, 2H), 1.70 (s, 2H), 1.63-1.55 (m, 2H), 1.54-1.41 (m, 12H). |
| 9 | 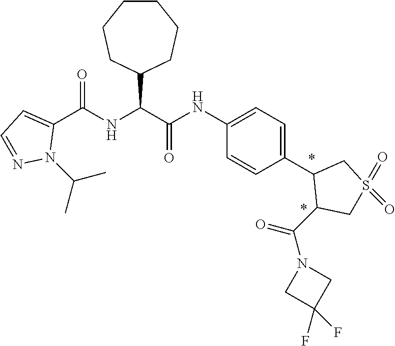 [SMILES: CC(C)n1nccc1C(=O)N[C@H](C(=O)Nc1ccc(C2CS(=O)(=O)CC2C(=O)N2CC(F)(F)C2)cc1)C1CCCCCC1] | 620.3 | ¹H NMR (400 MHz, Chloroform-d) δ 8.18 (s, 1H), 7.50 (dd, J = 8.9, 2.1 Hz, 3H), 7.19 (d, J = 8.3 Hz, 2H), 6.77 (d, J = 8.6 Hz, 1H), 6.56 (d, J = 2.1 Hz, 1H), 5.46-5.33 (m, 1H), 4.57-4.48 (m, 1H), 4.41-4.13 (m, 3H), 4.00-3.74 (m, 2H), 3.72-3.46 (m, 4H), 2.92-2.83 (m, 1H), 2.22-2.12 (m, 1H), 1.88-1.79 (m, 2H), 1.70 (s, 2H), 1.63-1.55 (m, 2H), 1.54-1.41 (m, 12H). |
| 10 | 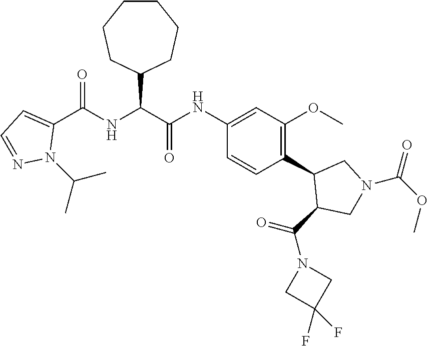 [SMILES: COC(=O)N1C[C@H](c2ccc(NC(=O)[C@@H](NC(=O)c3ccnn3C(C)C)C3CCCCCC3)cc2OC)[C@H](C(=O)N2CC(F)(F)C2)C1] | 659.3 | ¹H NMR (400 MHz, Chloroform-d) δ 8.20 (s, 1H), 7.65 (dd, J = 8.9, 2.1 Hz, 2H), 7.22 (d, J = 8.3 Hz, 1H), 6.91 (d, J = 8.6 Hz, 1H), 6.56 (d, J = 2.1 Hz, 1H), 5.46-5.33 (m, 1H), 4.57-4.48 (m, 1H), 4.26-4.13 (m, 3H), 4.00-3.81 (m, 2H), 3.76 (s, 3H), 3.71 (s, 3H), 3.64- 3.44 (m, 4H), 2.90- 2.83 (m, 1H), 2.21- 2.08 (m, 1H), 1.88- 1.79 (m, 2H), 1.70 (s, 2H), 1.63-1.55 (m, 2H), 1.54-1.41 (m, 12H). |
| 11 | 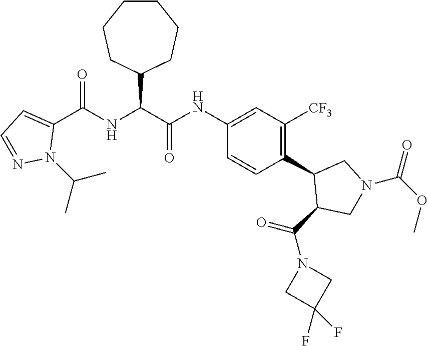 [SMILES: COC(=O)N1C[C@H](c2ccc(NC(=O)[C@@H](NC(=O)c3ccnn3C(C)C)C3CCCCCC3)cc2C(F)(F)F)[C@H](C(=O)N2CC(F)(F)C2)C1] | 697.3 | ¹H NMR (400 MHz, Chloroform-d) δ 8.20 (s, 1H), 7.85 (dd, J = 8.9, 2.1 Hz, 2H), 7.34 (d, J = 8.3 Hz, 1H), 6.91 (d, J = 8.6 Hz, 1H), 6.56 (d, J = 2.1 Hz, 1H), 5.46-5.33 (m, 1H), 4.57-4.48 (m, 1H), 4.26-4.13 (m, 3H), 4.00-3.81 (m, 2H), 3.71 (s, 3H), 3.64-3.44 (m, 4H), 2.90-2.83 (m, 1H), 2.21-2.08 (m, 1H), 1.88-1.79 (m, 2H), 1.70 (s, 2H), 1.63- 1.55 (m, 2H), 1.54- 1.41 (m, 12H). |
| 12 | 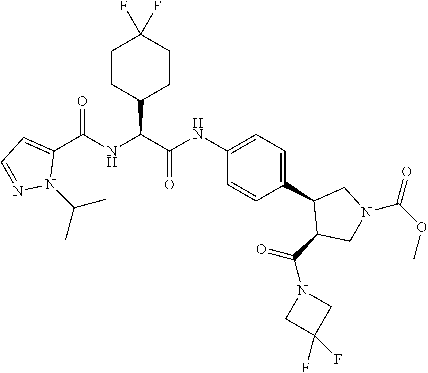 [SMILES: COC(=O)N1C[C@H](c2ccc(NC(=O)[C@@H](NC(=O)c3ccnn3C(C)C)C3CCC(F)(F)CC3)cc2)[C@H](C(=O)N2CC(F)(F)C2)C1] | 651.3 | ¹H NMR (400 MHz, Chloroform-d) δ 8.19 (s, 1H), 7.65 (dd, J = 8.9, 2.1 Hz, 3H), 7.22 (d, J = 8.3 Hz, 2H), 6.91 (d, J = 8.6 Hz, 2H), 6.56 (d, J = 2.1 Hz, 1H), 5.46-5.33 (m, 1H), 4.57-4.48 (m, 1H), 4.26-4.13 (m, 3H), 4.00-3.81 (m, 2H), 3.71 (s, 3H), 3.64-3.44 (m, 4H), 2.90-2.83 (m, 1H), 2.21-2.08 (m, 1H), 1.88-1.79 (m, 2H), 1.63-1.55 (m, 2H), 1.52-1.43 (m, 10H). |
| 13 | 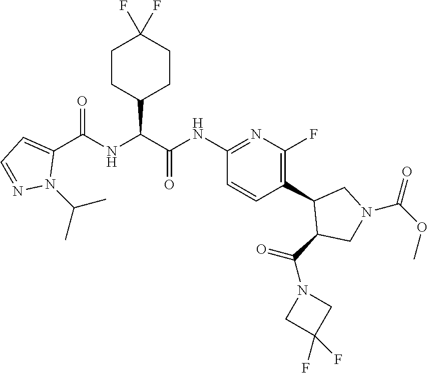 [SMILES: COC(=O)N1C[C@H](c2ccc(NC(=O)[C@@H](NC(=O)c3ccnn3C(C)C)C3CCC(F)(F)CC3)nc2F)[C@H](C(=O)N2CC(F)(F)C2)C1] | 670.3 | ¹H NMR (400 MHz, Chloroform-d) δ 8.19 (s, 1H), 7.67 (dd, J = 8.9, 2.1 Hz, 2H), 7.35- 7.22 (m, 2H), 6.76 (d, J = 2.1 Hz, 1H), 5.46- 5.33 (m, 1H), 4.57- 4.48 (m, 1H), 4.26- 4.13 (m, 3H), 4.00- 3.81 (m, 2H), 3.71 (s, 3H), 3.64-3.44 (m, 4H), 2.90-2.83 (m, 1H), 2.21-2.08 (m, 1H), 1.88-1.79 (m, 2H), 1.63-1.55 (m, 2H), 1.52-1.43 (m, 10H). |
| 14 | 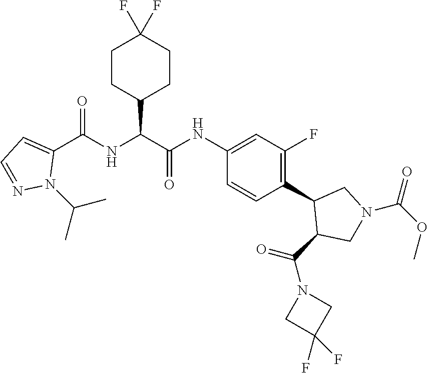 [SMILES: COC(=O)N1C[C@H](c2ccc(NC(=O)[C@@H](NC(=O)c3ccnn3C(C)C)C3CCC(F)(F)CC3)cc2F)[C@H](C(=O)N2CC(F)(F)C2)C1] | 669.3 | ¹H NMR (400 MHz, Chloroform-d) δ 8.19 (s, 1H), 7.75 (m, 2H), 7.40 (d, J = 8.3 Hz, 1H), 7.22 (d, J = 4.3 Hz, 1H), 6.91 (d, J = 8.6 Hz, 1H), 6.56 (d, J = 2.1 Hz, 1H), 5.46- 5.33 (m, 1H), 4.57- 4.48 (m, 1H), 4.26- 4.13 (m, 3H), 4.00- 3.81 (m, 2H), 3.71 (s, 3H), 3.64-3.44 (m, 4H), 2.90-2.83 (m, 1H), 2.21-2.08 (m, 1H), 1.88-1.79 (m, 2H), 1.63-1.55 (m, 2H), 1.52-1.43 (m, 10H). |
| 15 | 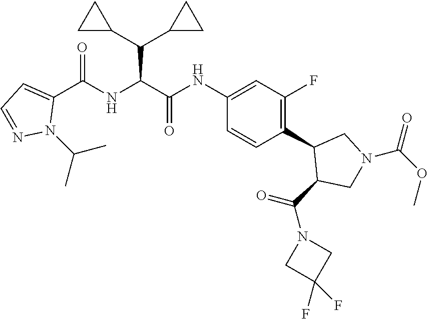 [SMILES: COC(=O)N1C[C@H](c2ccc(NC(=O)[C@@H](NC(=O)c3ccnn3C(C)C)C(C3CC3)C3CC3)cc2F)[C@H](C(=O)N2CC(F)(F)C2)C1] | 650.3 | ¹H NMR (400 MHz, Chloroform-d) δ 8.18 (s, 1H), 7.75 (m, 2H), 7.40 (d, J = 8.3 Hz, 1H), 7.22 (d, J = 4.3 Hz, 1H), 6.91 (d, J = 8.6 Hz, 1H), 6.56 (d, J = 2.1 Hz, 1H), 5.46- 5.33 (m, 1H), 4.57- 4.48 (m, 1H), 4.26- 4.13 (m, 3H), 4.00- 3.81 (m, 2H), 3.71 (s, 3H), 3.64-3.44 (m, 4H), 2.90-2.83 (m, 1H), 1.41-1.29 (m, 8H), 0.63-0.51 (m, 4H), 0.42-0.32 (m, 4H). |
| 16 | 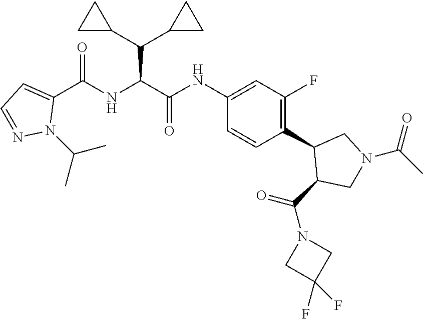 [SMILES: CC(=O)N1C[C@H](c2ccc(NC(=O)[C@@H](NC(=O)c3ccnn3C(C)C)C(C3CC3)C3CC3)cc2F)[C@H](C(=O)N2CC(F)(F)C2)C1] | 629.3 | ¹H NMR (400 MHz, Chloroform-d) δ 8.18 (s, 1H), 7.75 (m, 2H), 7.40 (d, J = 8.3 Hz, 1H), 7.22 (d, J = 4.3 Hz, 1H), 6.91 (d, J = 8.6 Hz, 1H), 6.56 (d, J= 2.1 Hz, 1H), 5.46- 5.33 (m, 1H), 4.57- 4.48 (m, 1H), 4.26- 4.13 (m, 3H), 4.00- 3.81 (m, 2H), 3.64- 3.44 (m, 4H), 2.90 2.83 (m, 1H), 2.41 (s, 3H), 1.41-1.29 (m, 8H), 0.63-0.51 (m, 4H), 0.42-0.32 (m, 4H). |
| 17 | 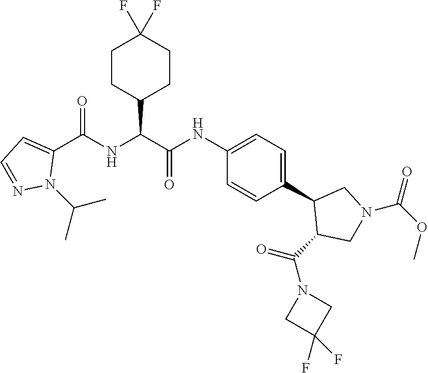 [SMILES: COC(=O)N1C[C@H](C(=O)N2CC(F)(F)C2)[C@@H](c2ccc(NC(=O)[C@@H](NC(=O)c3ccnn3C(C)C)C3CCC(F)(F)CC3)cc2)C1] | 651.3 | ¹H NMR (400 MHz, Chloroform-d) δ 8.19 (s, 1H), 7.65 (dd, J = 8.9, 2.1 Hz, 3H), 7.22 (d, J = 8.3 Hz, 2H), 6.91 (d, J = 8.6 Hz, 2H), 6.56 (d, J = 2.1 Hz, 1H), 5.46-5.33 (m, 1H), 4.57-4.48 (m, 1H), 4.24-4.13 (m, 3H), 4.02-3.81 (m, 2H), 3.71 (s, 3H), 3.64-3.44 (m, 4H), 2.90-2.83 (m, 1H), 2.21-2.08 (m, 1H), 1.88-1.79 (m, 2H), 1.63-1.55 (m, 2H), 1.52-1.43 (m, 10H). |
| 18 | 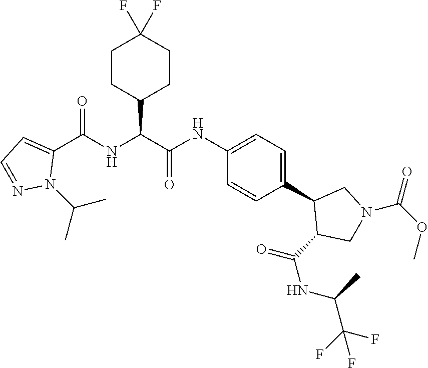 [SMILES: COC(=O)N1C[C@H](C(=O)N[C@@H](C)C(F)(F)F)[C@@H](c2ccc(NC(=O)[C@@H](NC(=O)c3ccnn3C(C)C)C3CCC(F)(F)CC3)cc2)C1] | 671.3 | ¹H NMR (400 MHz, Chloroform-d) δ 8.18 (s, 1H), 8.03 (d, J = 6.1 Hz, 1H), 7.65 (dd, J = 8.9, 2.1 Hz, 3H), 7.22 (d, J = 8.3 Hz, 2H), 6.91 (d, J = 8.6 Hz, 2H), 6.56 (d, J = 2.1 Hz, 1H), 5.76-5.53 (m, 1H), 5.46-5.33 (m, 1H), 4.57-4.48 (m, 1H), 4.24-4.13 (m, 3H), 4.02-3.81 (m, 2H), 3.71 (s, 3H), 2.90-2.83 (m, 1H), 2.21-2.08 (m, 1H), 1.88-1.79 (m, 2H), 1.63-1.55 (m, 2H), 1.52-1.37 (m, 9H). |
| 19 | 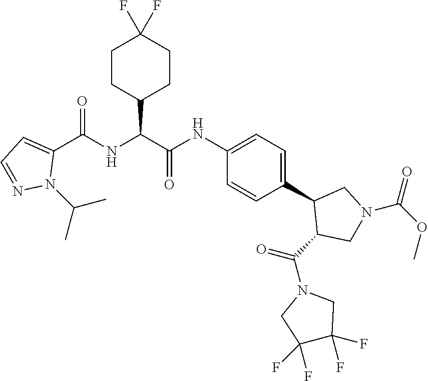 [SMILES: COC(=O)N1C[C@H](C(=O)N2CC(F)(F)C(F)(F)C2)[C@@H](c2ccc(NC(=O)[C@@H](NC(=O)c3ccnn3C(C)C)C3CCC(F)(F)CC3)cc2)C1] | 701.3 | ¹H NMR (400 MHz, Chloroform-d) δ 8.18 (s, 1H), 7.65 (dd, J = 8.9, 2.1 Hz, 3H), 7.22 (d, J = 8.3 Hz, 2H), 6.91 (d, J = 8.6 Hz, 2H), 6.56 (d, J = 2.1 Hz, 1H), 5.46-5.33 (m, 1H), 4.57-4.48 (m, 1H), 4.24-4.13 (m, 3H), 4.05-3.83 (m, 2H), 3.72 (s, 3H), 3.64-3.47 (m, 4H), 2.90-2.83 (m, 1H), 2.21-2.08 (m, 1H), 1.88-1.79 (m, 2H), 1.63-1.55 (m, 2H), 1.52-1.43 (m, 10H). |
| 20 | 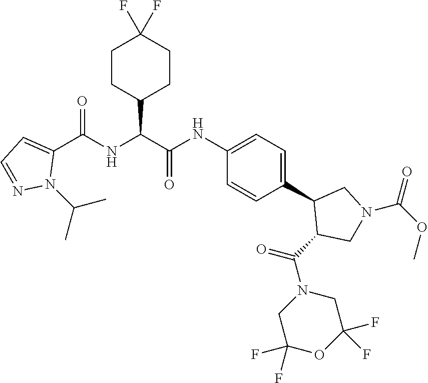 [SMILES: COC(=O)N1C[C@H](C(=O)N2CC(F)(F)OC(F)(F)C2)[C@@H](c2ccc(NC(=O)[C@@H](NC(=O)c3ccnn3C(C)C)C3CCC(F)(F)CC3)cc2)C1] | 717.3 | ¹H NMR (400 MHz, Chloroform-d) δ 8.18 (s, 1H), 7.65 (dd, J = 8.9, 2.1 Hz, 3H), 7.22 (d, J = 8.3 Hz, 2H), 6.91 (d, J = 8.6 Hz, 2H), 6.56 (d, J = 2.1 Hz, 1H), 5.46-5.33 (m, 1H), 4.57-4.48 (m, 1H), 4.24-4.13 (m, 3H), 4.05-3.83 (m, 2H), 3.71 (s, 3H), 3.68-3.45 (m, 4H), 2.90-2.83 (m, 1H), 2.21-2.08 (m, 1H), 1.88-1.79 (m, 2H), 1.63-1.55 (m, 2H), 1.52-1.37 (m, 10H). |
| 21 | 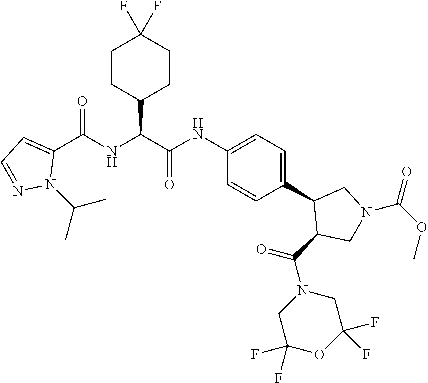 [SMILES: COC(=O)N1C[C@H](c2ccc(NC(=O)[C@@H](NC(=O)c3ccnn3C(C)C)C3CCC(F)(F)CC3)cc2)[C@H](C(=O)N2CC(F)(F)OC(F)(F)C2)C1] | 717.3 | ¹H NMR (400 MHz, Chloroform-d) δ 8.19 (s, 1H), 7.65 (dd, J = 8.9, 2.1 Hz, 3H), 7.22 (d, J = 8.3 Hz, 2H), 6.91 (d, J = 8.6 Hz, 2H), 6.56 (d, J = 2.1 Hz, 1H), 5.46-5.33 (m, 1H), 4.57-4.48 (m, 1H), 4.24-4.13 (m, 3H), 4.02-3.81 (m, 2H), 3.71 (s, 3H), 3.68-3.45 (m, 4H), 2.90-2.83 (m, 1H), 2.21-2.08 (m, 1H), 1.88-1.79 (m, 2H), 1.63-1.55 (m, 2H), 1.52-1.37 (m, 10H). |
| 22 | 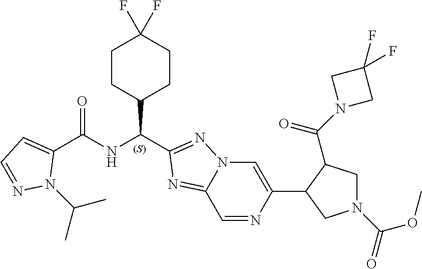 | 650.3 | ¹H NMR (400 MHz, Chloroform-d) δ 8.56 (s, 1H), 8.06 (s, 1H), 7.71(s, 1H), 7.65 (d, J = 2.1 Hz, 1H), 6.56 (d, J = 2.1 Hz, 1H), 5.46-5.33 (m, 1H), 4.57-4.48 (m, 1H), 4.24-4.13 (m, 3H), 4.02-3.81 (m, 2H), 3.71 (s, 3H), 3.64- 3.44 (m, 4H), 2.90- 2.83 (m, 1H), 2.21- 2.08 (m, 1H), 1.88- 1.79 (m, 2H), 1.63- 1.55 (m, 2H), 1.52- 1.43 (m, 10H). |
| 23 | 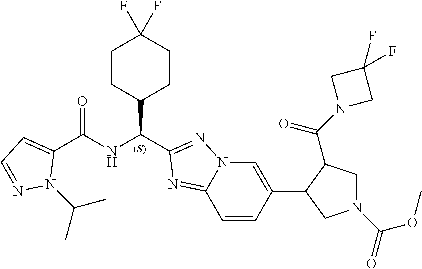 | 649.3 | ¹H NMR (400 MHz, Chloroform-d) δ 8.97 (s, 1H), 8.06 (s, 1H), 7.65 (d, J = 2.1 Hz, 1H), 7.46 (s, 1H), 7.38 (s, 1H), 6.56 (d, J = 2.1 Hz, 1H), 5.46- 5.33 (m, 1H), 4.57- 4.48 (m, 1H), 4.24- 4.13 (m, 3H), 4.02- 3.81 (m, 2H), 3.71 (s, 3H), 3.64-3.44 (m, 4H), 2.90-2.83 (m, 1H), 2.21-2.08 (m, 1H), 1.88-1.79 (m, 2H), 1.63-1.55 (m, 2H), 1.52-1.43 (m, 10H). |
| 24 | 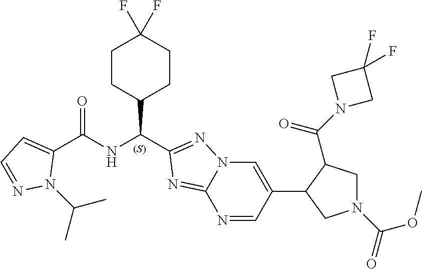 | 650.3 | ¹H NMR (400 MHz, Chloroform-d) δ 8.87 (s, 1H), 8.35(s, 1H), 8.06 (s, 1H), 7.65 (d, J = 2.1 Hz, 1H), 6.56 (d, J = 2.1 Hz, 1H), 5.46-5.33 (m, 1H), 4.57-4.48 (m, 1H), 4.24-4.13 (m, 3H), 4.02-3.81 (m, 2H), 3.71 (s, 3H), 3.64- 3.44 (m, 4H), 2.90- 2.83 (m, 1H), 2.21- 2.08 (m, 1H), 1.88- 1.79 (m, 2H), 1.63- 1.55 (m, 2H), 1.52- 1.43 (m, 10H). |
| 25 | 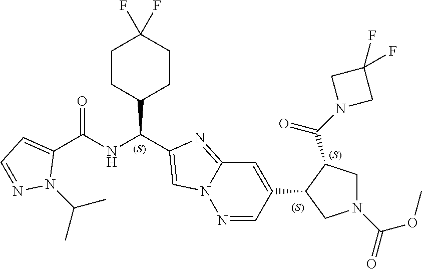 | 649.3 | ¹H NMR (400 MHz, Chloroform-d) δ 10.57 (s, 1H), 10.16(s, 1H), 9.87(s, 1H), 8.06 (s, 1H), 7.65 (d, J = 2.1 Hz, 1H), 6.56 (d, J = 2.1 Hz, 1H), 5.46- 5.33 (m, 1H), 4.57- 4.48 (m, 1H), 4.24- 4.13 (m, 3H), 4.02- 3.81 (m, 2H), 3.71 (s, 3H), 3.64-3.44 (m, 4H), 2.90-2.83 (m, 1H), 2.21-2.08 (m, 1H), 1.88-1.79 (m, 2H), 1.63-1.55 (m, 2H), 1.52-1.43 (m, 10H). |
| 26 | 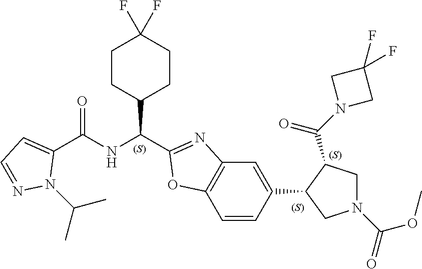 | 649.3 | ¹H NMR (400 MHz, Chloroform-d) δ 8.06 (s, 1H), 7.65-7.55 (m, 3H), 7.04(d, J = 4.8 Hz, 1H), 6.56 (d, J = 2.1 Hz, 1H), 5.46- 5.33 (m, 1H), 4.57- 4.48 (m, 1H), 4.24- 4.13 (m, 3H), 4.02- 3.81 (m, 2H), 3.71 (s, 3H), 3.64-3.44 (m, 4H), 2.90-2.83 (m, 1H), 2.21-2.08 (m, 1H), 1.88-1.79 (m, 2H), 1.63-1.55 (m, 2H), 1.52-1.43 (m, 10H). |
| 27 | 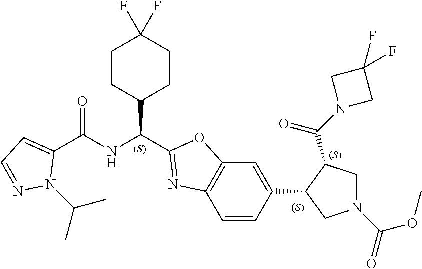 | 649.3 | ¹H NMR (400 MHz, Chloroform-d) δ 8.06 (s, 1H), 7.65 (d, J = 2.1 Hz, 1H), 7.43 (s, 1H), 7.33(s, 1H), 7.19(s, 1H), 6.56 (d, J = 2.1 Hz, 1H), 5.46- 5.33 (m, 1H), 4.57- 4.48 (m, 1H), 4.24- 4.13 (m, 3H), 4.02- 3.81 (m, 2H), 3.71 (s, 3H), 3.64-3.44 (m, 4H), 2.90-2.83 (m, 1H), 2.21-2.08 (m, 1H), 1.88-1.79 (m, 2H), 1.63-1.55 (m, 2H), 1.52-1.43 (m, 10H). |
| 28 | 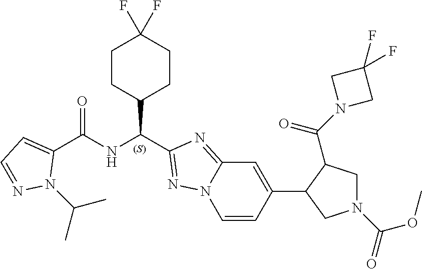 | 649.3 | ¹H NMR (400 MHz, Chloroform-d) δ 8.29 (s, 1H), 8.06 (s, 1H), 7.65 (d, J = 2.1 Hz, 1H), 7.33(d, J = 5.6Hz, 1H), 7.19(s, 1H), 6.56 (d, J = 2.1 Hz, 1H), 5.46-5.33 (m, 1H), 4.57-4.48 (m, 1H), 4.24-4.13 (m, 3H), 4.02-3.81 (m, 2H), 3.71 (s, 3H), 3.64-3.44 (m, 4H), 2.90-2.83 (m, 1H), 2.21-2.08 (m, 1H), 1.88-1.79 (m, 2H), |
| | | | 1.63-1.55 (m, 2H), |
| | | | 1.52-1.43 (m, 10H). |
| 29 | 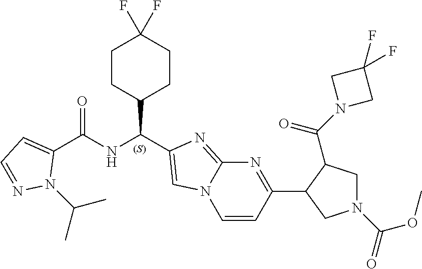 | 649.3 | ¹H NMR (400 MHz, Chloroform-d) δ 8.48 (s, 1H), 8.06 (s, 1H), 7.90(s, 1H), 7.65 (d, J = 2.1 Hz, 1H), 7.13(s, 1H), 6.56 (d, J = 2.1 Hz, 1H), 5.46- 5.33 (m, 1H), 4.57- 4.48 (m, 1H), 4.24- 4.13 (m, 3H), 4.02- 3.81 (m, 2H), 3.71 (s, 3H), 3.64-3.44 (m, 4H), 2.90-2.83 (m, 1H), 2.21-2.08 (m, 1H), 1.88-1.79 (m, 2H), 1.63-1.55 (m, 2H), 1.52-1.43 (m, 10H). |
| 30 | 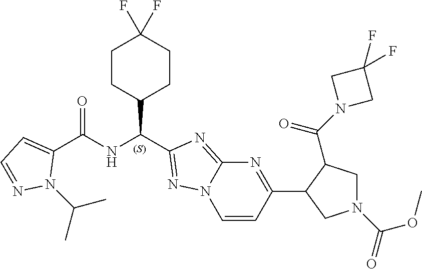 | 650.3 | ¹H NMR (400 MHz, Chloroform-d) δ 8.71 (s, 1H), 8.06 (s, 1H), 7.65 (d, J = 2.1 Hz, 1H), 7.20 (s, 1H), 6.56 (d, J = 2.1 Hz, 1H), 5.46-5.33 (m, 1H), 4.57-4.48 (m, 1H), 4.24-4.13 (m, 3H), 4.02-3.81 (m, 2H), 3.71 (s, 3H), 3.64- 3.44 (m, 4H), 2.90- 2.83 (m, 1H), 2.21- 2.08 (m, 1H), 1.88- 1.79 (m, 2H), 1.63- 1.55 (m, 2H), 1.52- 1.43 (m, 10H). |
| 31 | 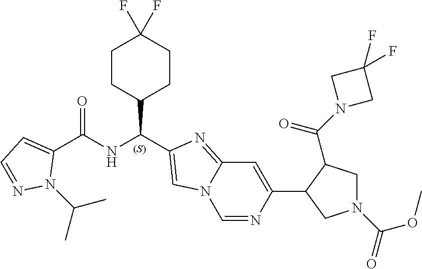 | 649.3 | ¹H NMR (400 MHz, Chloroform-d) δ 8.99 (s, 1H), 8.06 (s, 1H), 7.65 (d, J = 2.1 Hz, 1H), 7.05 (s, 1H), 6.89(s, 1H), 6.56 (d, J = 2.1 Hz, 1H), 5.46- 5.33 (m, 1H), 4.57- 4.48 (m, 1H), 4.24- 4.13 (m, 3H), 4.02- 3.81 (m, 2H), 3.71 (s, 3H), 3.64-3.44 (m, 4H), 2.90-2.83 (m, 1H), 2.21-2.08 (m, 1H), 1.88-1.79 (m, 2H), 1.63-1.55 (m, |
| | | | 2H), 1.52-1.43 (m, |
| | | | 10H). |
| 32 | 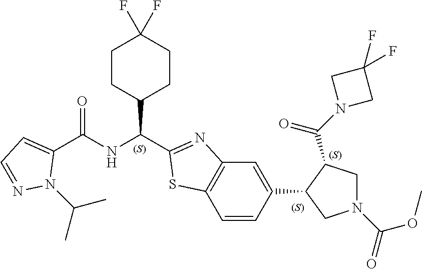 | 665.3 | ¹H NMR (400 MHz, Chloroform-d) δ 9.87 (d, J-7.6Hz, 1H), 8.06 (s, 1H), 7.65-7.55 (m, 2H), 7.24(d, J =7.6Hz, 1H), 6.56 (d, J = 2.1 Hz, 1H), 5.46 5.33 (m, 1H), 4.57- 4.48 (m, 1H), 4.24- 4.13 (m, 3H), 4.02- 3.81 (m, 2H), 3.71 (s, 3H), 3.64-3.44 (m, 4H), 2.90-2.83 (m, 1H), 2.21-2.08 (m, 1H), 1.88-1.79 (m, 2H), 1.63-1.55 (m, |
| | | | 2H), 1.52-1.43 (m, |
| | | | 10H). |
| 33 | 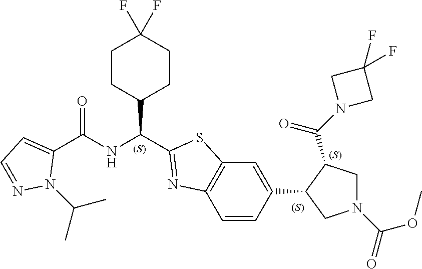 | 665.3 | ¹H NMR (400 MHz, Chloroform-d) δ 8.06 (s, 1H), 7.63-7.55 (m, 3H), 7.24-7.21 (m, 1H), 6.56 (d, J = 2.1 Hz, 1H), 5.46-5.33 (m, 1H), 4.57-4.48 (m, 1H), 4.24-4.13 (m, 3H), 4.02-3.81 (m, 2H), 3.71 (s, 3H), 3.64-3.44 (m, 4H), 2.90-2.83 (m, 1H), 2.21-2.08 (m, 1H), 1.88-1.79 (m, 2H), 1.63-1.55 (m, 2H), 1.52-1.43 (m, 10H). |
| 34 | 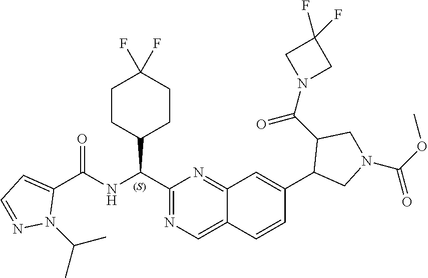 | 660.3 | ¹H NMR (400 MHz, Chloroform-d) δ 9.11 (s, 1H), 8.06-8.03 (m, 2H), 7.77(s, 1H), 7.63 (d, J = 2.1 Hz, 1H), 7.26 (d, J = 4.8 Hz, 1H), 6.56 (d, J = 2.1 Hz, 1H), 5.46-5.33 (m, 1H), 4.57-4.48 (m, 1H), 4.24-4.13 (m, 3H), 4.02-3.81 (m, 2H), 3.71 (s, 3H), 3.64-3.44 (m, 4H), 2.90-2.83 (m, 1H), 2.21-2.08 (m, 1H), 1.88-1.79 (m, 2H), 1.63-1.55 (m, 2H), 1.52-1.43 (m, 10H). |
| 35 | 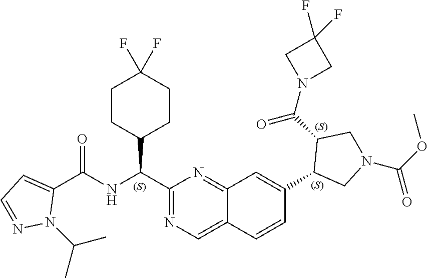 | 660.3 | ¹H NMR (400 MHz, Chloroform-d) δ 9.11 (s, 1H), 8.06-8.03 (m, 2H), 7.77(s, 1H), 7.63 (d, J = 2.1 Hz, 1H), 7.26 (d, J-4.8Hz, 1H), 6.56 (d, J = 2.1 Hz, 1H), 5.46-5.33 (m, 1H), 4.57-4.48 (m, 1H), 4.24-4.13 (m, 3H), 4.02-3.81 (m, 2H), 3.71 (s, 3H), 3.64-3.44 (m, 4H), 2.90-2.83 (m, 1H), 2.21-2.08 (m, 1H), 1.88-1.79 (m, 2H), 1.63-1.55 (m, 2H), 1.52-1.43 (m, 10H). |
| 36 | 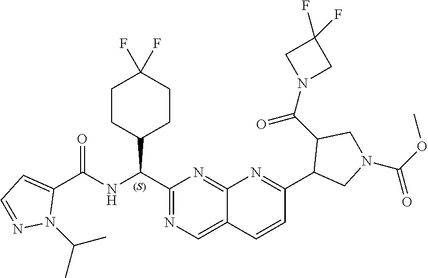 | 661.3 | ¹H NMR (400 MHz, Chloroform-d) δ 9.13 (s, 1H), 8.12 (d, J = 4.8 Hz, 1H), 8.05 (s, 1H), 7.55 (d, J = 4.8 Hz, 1H), 7.63 (d, J = 2.1 Hz, 1H), 6.56 (d, J = 2.1 Hz, 1H), 5.46- 5.33 (m, 1H), 4.57- 4.48 (m, 1H), 4.24- 4.13 (m, 3H), 4.02- 3.81 (m, 2H), 3.71 (s, 3H), 3.64-3.44 (m, 4H), 2.90-2.83 (m, 1H), 2.21-2.08 (m, 1H), 1.88-1.79 (m, 2H), 1.63-1.55 (m, |
| | | | 2H), 1.52-1.43 (m, |
| | | | 10H). |
| 37 | 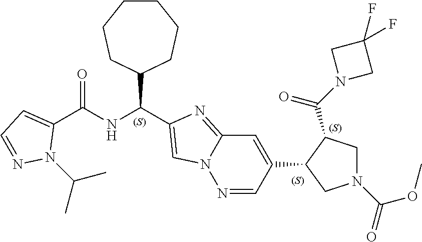 | 627.3 | ¹H NMR (400 MHz, Chloroform-d) δ 10.57 (s, 1H), 10.16 (s, 1H), 9.87 (s, 1H), 8.08 (s, 1H), 7.65 (d, J = 2.1 Hz, 1H), 6.56 (d, J = 2.1 Hz, 1H), 5.46- 5.33 (m, 1H), 4.57- 4.48 (m, 1H), 4.26- 4.13 (m, 3H), 4.00- 3.74 (m, 2H), 3.71 (s, 3H), 3.69-3.44 (m, 4H), 2.92-2.83 (m, 1H), 2.22-2.08 (m, 1H), 1.88-1.79 (m, |
| | | | 2H), 1.70 (s, 2H), 1.63- |
| | | | 1.55 (m, 2H), 1.54- |
| | | | 1.41 (m, 12H). |
| 38 | 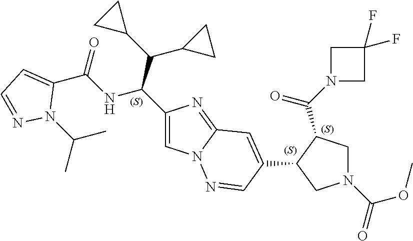 | 625.3 | ¹H NMR (400 MHz, Chloroform-d) δ 10.57 (s, 1H), 10.16(s, 1H), 9.87(s, 1H), 8.09 (s, 1H), 7.65 (d, J = 2.1 Hz, 1H), 6.56 (d, J = 2.1 Hz, 1H), 5.46- 5.33 (m, 1H), 4.57- 4.48 (m, 1H), 4.26- 4.13 (m, 3H), 4.00- 3.81 (m, 2H), 3.71 (s, 3H), 3.64-3.44 (m, 4H), 2.90-2.83 (m, 1H), 1.41-1.29 (m, 8H), 0.63-0.51 (m, 4H), 0.42-0.32 (m, 4H). |
| 39 | 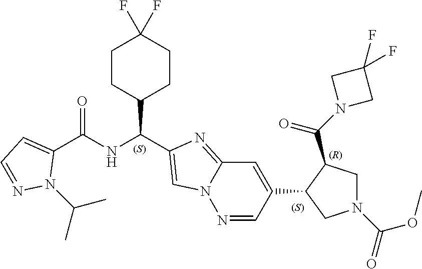 | 649.3 | ¹H NMR (400 MHz, Chloroform-d) δ 10.57 (s, 1H), 10.16 (s, 1H), 9.87 (s, 1H), 8.06 (s, 1H), 7.65 (d, J = 2.1 Hz, 1H), 6.56 (d, J = 2.1 Hz, 1H), 5.46- 5.33 (m, 1H), 4.57- 4.48 (m, 1H), 4.26- 4.13 (m, 3H), 4.00- 3.74 (m, 2H), 3.71 (s, 3H), 3.69-3.44 (m, 4H), 2.90-2.82 (m, 1H), 2.22-2.08 (m, 1H), 1.88-1.79 (m, 2H), 1.70 (s, 2H), 1.63- |
| | | | 1.55 (m, 2H), 1.54- |
| | | | 1.41 (m, 12H). |
| 40 | 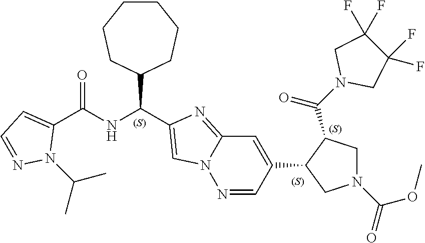 | 677.3 | ¹H NMR (400 MHz, Chloroform-d) δ 10.57 (s, 1H), 10.16 (s, 1H), 9.87 (s, 1H), 8.07 (s, 1H), 7.65 (d, J = 2.1 Hz, 1H), 6.56 (d, J = 2.1 Hz, 1H), 5.46- 5.33 (m, 1H), 4.57- 4.48 (m, 1H), 4.26- 4.13 (m, 3H), 4.03- 3.71 (m, 9H), 2.92- 2.83 (m, 1H), 2.22- 2.08 (m, 1H), 1.88- 1.79 (m, 2H), 1.70 (s, 2H), 1.63-1.55 (m, |
| | | | 2H), 1.54-1.41 (m, |
| | | | 12H). |
| 41 | 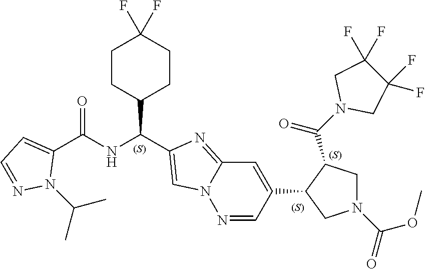 | 699.3 | ¹H NMR (400 MHz, Chloroform-d) δ 10.57 (s, 1H), 10.16 (s, 1H), 9.87 (s, 1H), 8.06 (s, 1H), 7.65 (d, J = 2.1 Hz, 1H), 6.56 (d, J = 2.1 Hz, 1H), 5.46- 5.33 (m, 1H), 4.57- 4.48 (m, 1H), 4.24- 4.13 (m, 3H), 4.05- 3.83 (m, 2H), 3.72 (s, 3H), 3.64-3.47 (m, 4H), 2.90-2.83 (m, 1H), 2.21-2.08 (m, 1H), 1.88-1.79 (m, 2H), 1.63-1.55 (m, |
| | | | 2H), 1.52-1.43 (m, |
| | | | 10H). |
| 42 | 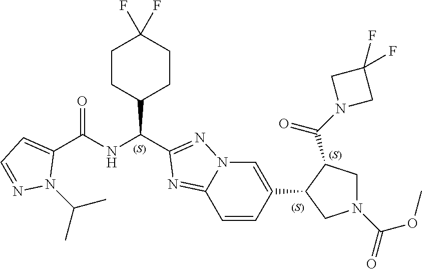 | 649.3 | ¹H NMR (400 MHz, Chloroform-d) δ 8.29 (s, 1H), 8.06 (s, 1H), 7.65 (d, J = 2.1 Hz, 1H), 7.33(d, J = 5.6 Hz, 1H), 7.19(s, 1H), 6.56 (d, J = 2.1 Hz, 1H), 5.46-5.33 (m, 1H), 4.57-4.48 (m, 1H), 4.24-4.13 (m, 3H), 4.02-3.81 (m, 2H), 3.71 (s, 3H), 3.64-3.44 (m, 4H), 2.90-2.83 (m, 1H), 2.21-2.08 (m, 1H), 1.88-1.79 (m, 2H), |
| | | | 1.63-1.55 (m, 2H), |
| | | | 1.52-1.43 (m, 10H). |
| 43 | 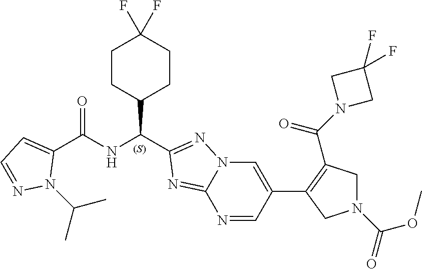 | 647.3 | ¹H NMR (400 MHz, Chloroform-d) δ 8.87 (s, 1H), 8.35 (s, 1H), 8.06 (s, 1H), 7.65 (d, J = 2.1 Hz, 1H), 6.56 (d, J = 2.1 Hz, 1H), 5.46-5.33 (m, 1H), 4.57-4.48 (m, 1H), 4.33-4.30(m, 2H), 4.24-4.13 (m, 3H), 4.03-3.99(m, 2H), 3.71 (s, 3H), 2.90-2.83 (m, 1H), 2.21-2.08 (m, 1H), 1.88-1.79 (m, 2H), 1.63-1.55 (m, 2H), 1.52-1.43 (m, 10H). |
| 44 | 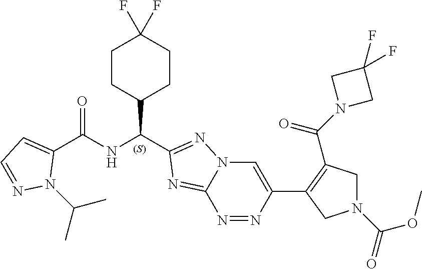 | 647.3 | ¹H NMR (400 MHz, Chloroform-d) δ 8.87 (s, 1H), 8.35 (s, 1H), 8.06 (s, 1H), 7.65 (d, J = 2.1 Hz, 1H), 6.56 (d, J = 2.1 Hz, 1H), 5.46-5.33 (m, 1H), 4.57-4.48 (m, 1H), 4.33-4.30(m, 2H), 4.24-4.13 (m, 3H), 4.03-3.99(m, 2H), 3.71 (s, 3H), 2.90-2.83 (m, 1H), 2.21-2.08 (m, 1H), 1.88-1.79 (m, 2H), 1.63-1.55 (m, 2H), 1.52-1.43 (m, 10H). |
| 45 | 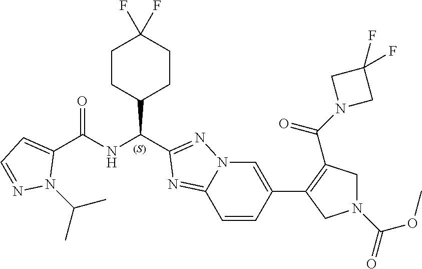 | 647.3 | ¹H NMR (400 MHz, Chloroform-d) δ 8.29 (s, 1H), 8.06 (s, 1H), 7.65 (d, J = 2.1 Hz, 1H), 7.33 (d, J = 5.6 Hz, 1H), 7.19 (s, 1H), 6.56 (d, J = 2.1 Hz, 1H), 5.46-5.33 (m, 1H), 4.57-4.48 (m, 1H), 4.33-4.30 (m, 2H), 4.24-4.13 (m, 3H), 4.03-3.99 (m, 2H), 3.71 (s, 3H), 2.90- 2.83 (m, 1H), 2.21- 2.08 (m, 1H), 1.88- 1.79 (m, 2H), 1.63- |
| | | | 1.55 (m, 2H), 1.52- |
| | | | 1.43 (m, 10H). |
| 46 | 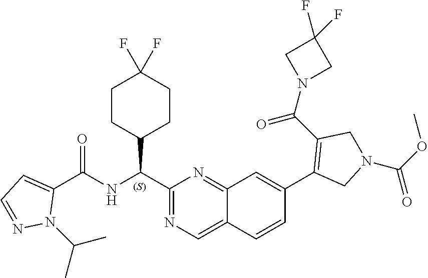 | 658.3 | ¹H NMR (400 MHz, Chloroform-d) δ 9.11 (s, 1H), 8.06-8.03 (m, 2H), 7.77 (s, 1H), 7.63 (d, J = 2.1 Hz, 1H), 7.26 (d, J = 4.8 Hz, 1H), 6.56 (d, J = 2.1 Hz, 1H), 5.46-5.33 (m, 1H), 4.57-4.48 (m, 1H), 4.33-4.30 (m, 2H), 4.24-4.13 (m, 3H), 4.03-3.99 (m, 2H), 3.71 (s, 3H), 2.90- 2.83 (m, 1H), 2.21- 2.08 (m, 1H), 1.88- 1.79 (m, 2H), 1.63 1.55 (m, 2H), 1.52- 1.43 (m, 10H). |
| 47 | 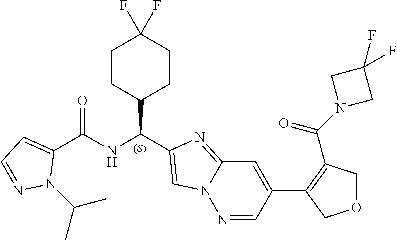 | 590.2 | ¹H NMR (400 MHz, Chloroform-d) δ 10.57 (s, 1H), 10.16 (s, 1H), 9.87(s, 1H), 8.06 (s, 1H), 7.65 (d, J = 2.1 Hz, 1H), 6.56 (d, J = 2.1 Hz, 1H), 5.46- 5.33 (m, 1H), 4.58- 4.48 (m, 3H), 4.32- 4.28(m, 1H), 4.26- 4.13 (m, 3H), 4.00- 3.74 (m, 2H), 2.90- 2.82 (m, 1H), 2.22- 2.08 (m, 1H), 1.88- 1.79 (m, 2H), 1.70 (s, |
| | | | 2H), 1.63-1.55 (m, |
| | | | 2H), 1.53-1.41 (m, |
| | | | 12H). |
| 48 | 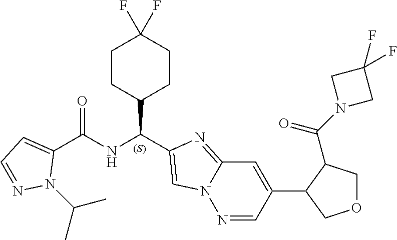 | 592.3 | ¹H NMR (400 MHz, Chloroform-d) δ 10.57 (s, 1H), 10.16 (s, 1H), 9.87(s, 1H), 8.06 (s, 1H), 7.65 (d, J = 2.1 Hz, 1H), 6.56 (d, J = 2.1 Hz, 1H), 5.46- 5.33 (m, 1H), 4.57- 4.48 (m, 1H), 4.26- 4.13 (m, 3H), 4.00- 3.74 (m, 2H), 3.82- 3.80(m, 2H), 3.69- 3.54 (m, 2H), 2.90- 2.82 (m, 1H), 2.22 2.08 (m, 1H), 1.88- |
| | | | 1.79 (m, 2H), 1.70 (s, |
| | | | 2H), 1.63-1.55 (m, |
| | | | 2H), 1.54-1.40 (m, |
| | | | 12H). |
| 49 | 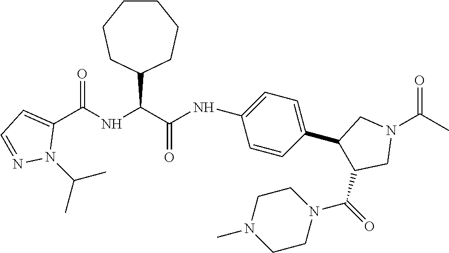 | 620.4 | ¹H NMR (400 MHz, Chloroform-d) δ 8.18 (s, 1H), 7.50 (dd, J = 8.9, 2.1 Hz, 3H), 7.19 (d, J = 8.3 Hz, 2H), 6.77 (d, J = 8.6 Hz, 1H), 6.56 (d, J = 2.1 Hz, 1H), 5.46-5.33 (m, 1H), 4.57-4.48 (m, 1H), 4.26-4.13 (m, 1H), 3.69-3.44 (m, 8H), 2.92-2.83 (m, 1H), 2.43-2.39 (m, 4H), 2.35 (s, 3H), 2.22- 2.12 (m, 1H), 2.10 (s, 3H), 1.88-1.79 (m, |
| | | | 2H), 1.70 (s, 2H), 1.63- |
| | | | 1.55 (m, 2H), 1.54- |
| | | | 1.41 (m, 12H). |
| 50 | 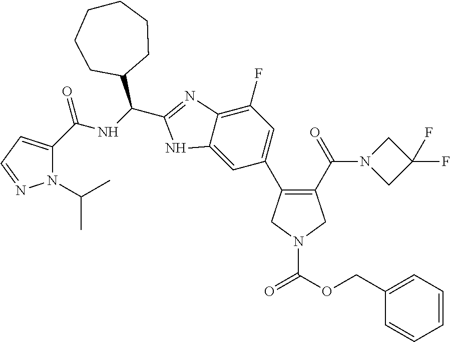 [SMILES: CC(C)n1nccc1C(=O)N[C@H](c1nc2c(F)cc(C3=C(C(=O)N4CC(F)(F)C4)CN(C(=O)OCc4ccccc4)C3)cc2[nH]1)C1CCCCCC1] | 718.3 | ¹H NMR (400 MHz, Chloroform-d) δ 8.39 (s, 1H), 8.35 (d, J = 6.4 Hz, 1H), 7.49-7.44 (m, 2H), 7.40-7.33 (m, 5H), 7.33-7.25 (m, 1H), 6.88 (d, J = 3.7 Hz, 1H), 5.14 (d, J = 0.9 Hz, 2H), 4.94- 4.90 (m, 1H), 4.72 (m, 1H), 4.55 (s, 1H), 4.48 (s, 1H), 4.07-3.97 (m, 5H), 2.28 (h, J = 7.5 Hz, 1H), 1.61-1.11 (m, 18H). |
| 51 | 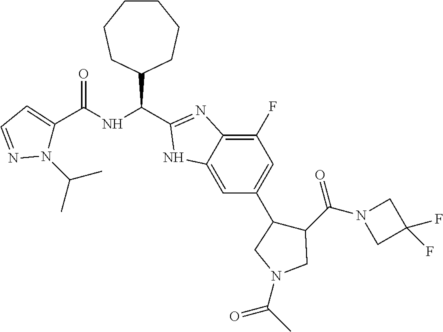 [SMILES: CC(=O)N1CC(C(=O)N2CC(F)(F)C2)C(c2cc(F)c3nc([C@@H](NC(=O)c4ccnn4C(C)C)C4CCCCCC4)[nH]c3c2)C1] | 628.3 | ¹H NMR (400 MHz, Chloroform-d) δ 8.37 (s, 1H), 8.35 (d, J = 6.4 Hz, 1H), 7.47 (d, J = 3.7 Hz, 1H), 7.31 (d, J = 2.1 Hz, 1H), 7.16- 7.11 (m, 1H), 6.88 (d, J = 3.7 Hz, 1H), 4.98 (dd, J = 7.5, 6.4 Hz, 1H), 4.72-4.65 (m, 1H), 4.04-4.00 (m, 3H), 3.95 (s, 1H), 3.88- 3.83 (m, 1H), 3.64- 3.60(m, 2H), 3.50- 3.43 (m, 2H), 3.14- 3.11 (m, 1H), 2.22- 2.11 (m, 4H), 1.71- 1.50 (m, 8H) , 1.46- 1.32 (m, 11H). |
| 52 | 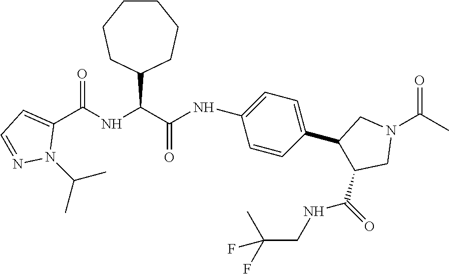 | 615.3 | ¹H NMR (400 MHz, Chloroform-d) δ 8.18 (s, 1H), 7.50 (dd, J = 8.9, 2.1 Hz, 3H), 7.19 (d, J = 8.3 Hz, 2H), 6.77 (d, J = 8.6 Hz, 1H), 6.56-6.51 (m, 2H), 5.46-5.33 (m, 1H), 4.57-4.48 (m, 1H), 4.26-4.13 (m, 3H), 4.00-3.74 (m, 2H), 3.55-3.52(m, 2H), 2.92-2.83 (m, 1H), 2.22-2.12 (m, 1H), 2.10 (s, 3H), 1.88- 1.79 (m, 5H), 1.70 (s, 2H), 1.63-1.55 (m, |
| | | | 2H), 1.54-1.41 (m, |
| | | | 12H). |
| 53 | 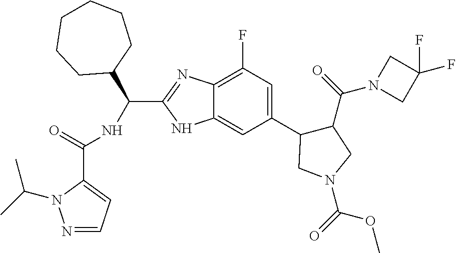 [SMILES: COC(=O)N1CC(C(=O)N2CC(F)(F)C2)C(c2cc(F)c3nc([C@@H](NC(=O)c4ccnn4C(C)C)C4CCCCCC4)[nH]c3c2)C1] | 644.3 | ¹H NMR (400 MHz, Chloroform-d) δ 8.37 (s, 1H), 8.35 (d, J = 6.4 Hz, 1H), 7.47 (d, J = 3.7 Hz, 1H), 7.31 (d, J = 2.1 Hz, 1H), 7.16- 7.11 (m, 1H), 6.88 (d, J = 3.7 Hz, 1H), 4.98 (dd, J = 7.5, 6.4 Hz, 1H), 4.72-4.65 (m, 1H), 4.04-4.00 (m, 3H), 3.95 (s, 1H), 3.88- 3.83 (m, 1H), 3.64- 3.60(m, 5H), 3.50- 3.43 (m, 2H), 3.14- 3.11 (m, 1H), 2.22- |
| | | | 2.11 (m, 1H), 1.71- |
| | | | 1.50 (m, 8H) , 1.46- |
| | | | 1.32 (m, 11H). |
| 54 | 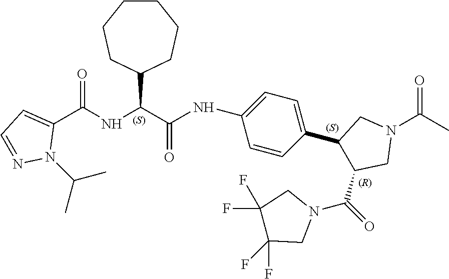 | 663.3 | ¹H NMR (400 MHz, Chloroform-d) δ 8.18 (s, 1H), 7.50 (dd, J = 8.9, 2.1 Hz, 3H), 7.19 (d, J = 8.3 Hz, 2H), 6.77 (d, J = 8.6 Hz, 1H), 6.56 (d, J = 2.1 Hz, 1H), 5.46-5.33 (m, 1H), 4.57-4.48 (m, 1H), 4.26-4.13 (m, 3H), 4.00-3.74 (m, 6H) , 2.92-2.83 (m, 1H), 2.22-2.12 (m, 1H), 2.10 (s, 3H), 1.88-1.79 (m, 2H), 1.70 (s, 2H), 1.63- 1.55 (m, 2H), 1.54- 1.41 (m, 12H). |
| 55 | 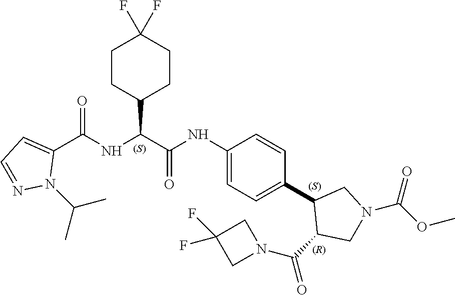 | 651.3 | ¹H NMR (400 MHz, Chloroform-d) δ 8.19 (s, 1H), 7.65 (dd, J = 8.9, 2.1 Hz, 3H), 7.22 (d, J = 8.3 Hz, 2H), 6.91 (d, J = 8.6 Hz, 2H), 6.56 (d, J = 2.1 Hz, 1H), 5.46-5.33 (m, 1H), 4.57-4.48 (m, 1H), 4.26-4.13 (m, 3H), 4.00-3.81 (m, 2H), 3.71 (s, 3H), 3.64-3.44 (m, 4H), 2.90-2.83 (m, 1H), 2.21-2.08 (m, 1H), 1.88-1.79 (m, 2H), 1.63-1.55 (m, 2H), 1.52-1.43 (m, 10H). |
| 56 | 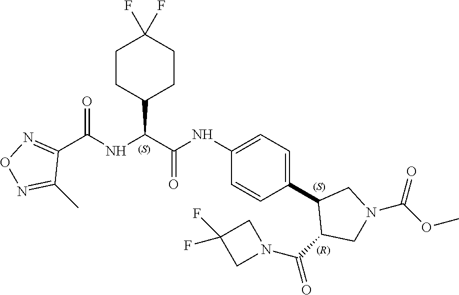 | 625.2 | ¹H NMR (400 MHz, Chloroform-d) δ 8.19 (s, 1H), 7.65 (dd, J = 8.9, 2.1 Hz, 3H), 7.22 (d, J = 8.3 Hz, 2H), 6.91 (d, J = 8.6 Hz, 2H), 6.56 (d, J = 2.1 Hz, 1H), 5.46-5.33 (m, 1H), 4.57-4.48 (m, 1H), 4.26-4.13 (m, 3H), 4.00-3.81 (m, 2H), 3.71 (s, 3H), 3.64-3.44 (m, 4H), 2.90-2.83 (m, 1H), 2.21-2.08 (m, 1H), 1.88-1.79 (m, 2H), 1.63-1.55 (m, 2H), 1.52-1.43 (m, 10H). |
| 57 | 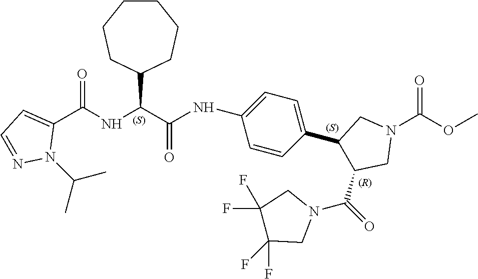 | 679.3 | ¹H NMR (400 MHz, Chloroform-d) δ 10.57 (s, 1H), 10.16 (s, 1H), 9.87(s, 1H), 8.08 (s, 1H), 7.65 (d, J = 2.1 Hz, 1H), 6.56 (d, J = 2.1 Hz, 1H), 5.46- 5.33 (m, 1H), 4.57- 4.48 (m, 1H), 4.26- 4.13 (m, 3H), 4.00- 3.74 (m, 6H), 3.71 (s, 3H), 2.92-2.83 (m, 1H), 2.22-2.08 (m, 1H), 1.88-1.79 (m, 2H), 1.70 (s, 2H), 1.63- 1.55 (m, 2H), 1.54- 1.41 (m, 12H). |
| 58 | 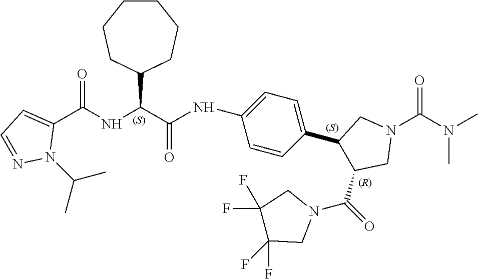 | 692.4 | ¹H NMR (400 MHz, Chloroform-d) δ 10.57 (s, 1H), 10.16(s, 1H), 9.87 (s, 1H), 8.08 (s, 1H), 7.65 (d, J = 2.1 Hz, 1H), 6.56 (d, J = 2.1 Hz, 1H), 5.46- 5.33 (m, 1H), 4.57- 4.48 (m, 1H), 4.26- 4.13 (m, 3H), 4.00- 3.74 (m, 6H), 2.92- 2.83 (m, 7H), 2.22- 2.08 (m, 1H), 1.88- 1.79 (m, 2H), 1.70 (s, 2H), 1.63-1.55 (m, 2H), 1.54-1.41 (m, 12H). |
| 59 | 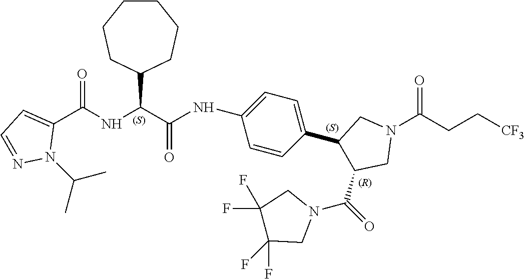 | 745.3 | ¹H NMR (400 MHz, Chloroform-d) δ 10.57 (s, 1H), 10.16(s, 1H), 9.87 (s, 1H), 8.08 (s, 1H), 7.65 (d, J = 2.1 Hz, 1H), 6.56 (d, J = 2.1 Hz, 1H), 5.46- 5.33 (m, 1H), 4.57- 4.48 (m, 1H), 4.26- 4.13 (m, 3H), 4.00- 3.74 (m, 6H), 2.92- 2.83 (m, 1H), 2.65- 2.61(m, 2H), 2.46- 2.43(m, 2H), 2.22- 2.08 (m, 1H), 1.88- 1.79 (m, 2H), 1.70 (s, 2H), 1.63-1.55 (m, 2H), 1.54-1.41 (m, 12H). |
| 60 | 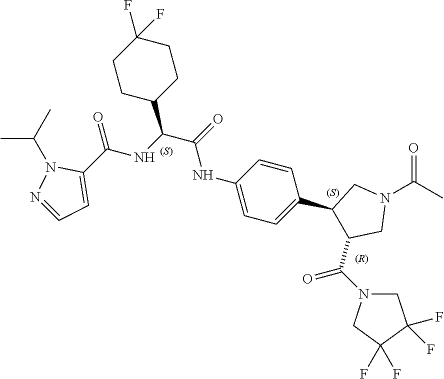 | 685.3 | ¹H NMR (400 MHz, Chloroform-d) δ 10.57 (s, 1H), 10.16 (s, 1H), 9.87 (s, 1H), 8.06 (s, 1H), 7.65 (d, J = 2.1 Hz, 1H), 6.56 (d, J = 2.1 Hz, 1H), 5.46- 5.33 (m, 1H), 4.57- 4.48 (m, 1H), 4.24- 4.13 (m, 3H), 4.05- 3.83 (m, 2H), 3.64- 3.47 (m, 4H), 2.90- 2.83 (m, 1H), 2.21- 2.08 (m, 4H), 1.88- 1.79 (m, 2H), 1.63- 1.55 (m, 2H), 1.52- 1.43 (m, 10H). |
| 61 | 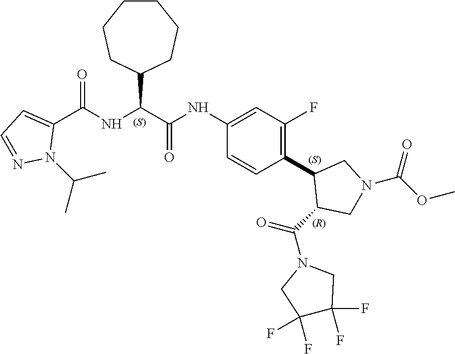 | 697.3 | ¹H NMR (400 MHz, Chloroform-d) δ 10.57 (s, 1H), 9.87- 9.83 (m, 2H), 8.08 (s, 1H), 6.56 (d, J = 2.1 Hz, 1H), 5.46-5.33 (m, 1H), 4.57-4.48 (m, 1H), 4.26-4.13 (m, 3H), 4.00-3.74 (m, 6H), 3.71 (s, 3H), 2.92-2.83 (m, 1H), 2.22-2.08 (m, 1H), 1.88-1.79 (m, 2H), 1.70 (s, 2H), 1.63- 1.55 (m, 2H), 1.54- 1.41 (m, 12H). |
| Cis- 62 | 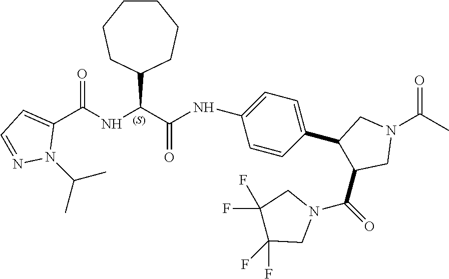 | 663.3 | 1H NMR (400 MHz, Chloroform-d) δ 8.18 (s, 1H), 7.50 (dd, J = 8.9, 2.1 Hz, 3H), 7.19 (d, J = 8.3 Hz, 2H), 6.77 (d, J = 8.6 Hz, 1H), 6.56 (d, J = 2.1 Hz, 1H), 5.46-5.33 (m, 1H), 4.57-4.48 (m, 1H), 4.26-4.13 (m, 3H), 4.00-3.74 (m, 6H) , 2.92-2.83 (m, 1H), 2.22-2.12 (m, 1H), 2.10 (s, 3H), 1.88-1.79 (m, 2H), 1.70 (s, 2H), 1.63- 1.55 (m, 2H), 1.54- 1.41 (m, 12H). |
| 63 | 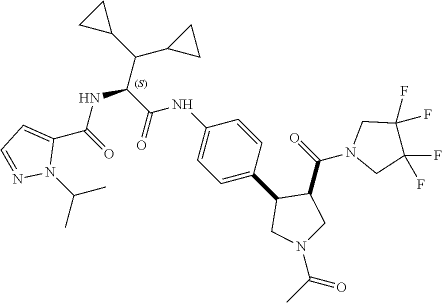 | 661.3 | ¹H NMR (400 MHz, Chloroform-d) δ 8.18 (s, 1H), 7.50 (dd, J = 8.9, 2.1 Hz, 3H), 7.19 (d, J = 8.3 Hz, 2H), 6.77 (d, J = 8.6 Hz, 1H), 6.56 (d, J = 2.1 Hz, 1H), 5.46-5.33 (m, 1H), 4.57-4.48 (m, 1H), 4.26-4.13 (m, 3H), 4.00-3.74 (m, 6H) , 2.92-2.83 (m, 1H), 2.22-2.12 (m, 1H), 2.10 (s, 3H), 1.88-1.79 (m, 2H), 1.70 (s, 2H), 1.63- 1.55 (m, 2H), 0.91- 0.72 (m, 3H), 0.69- 0.61(m, 1H), 0.58- |
| | | | 0.50(m, 3H), 0.42- |
| | | | 0.37(m, 2H). |
| 64 | 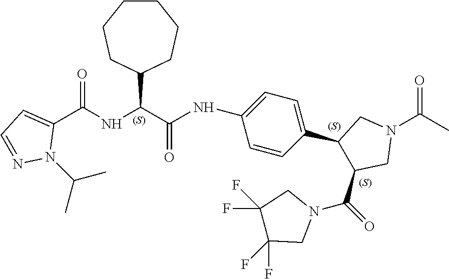 | 663.3 | ¹H NMR (400 MHz, Chloroform-d) δ 8.18 (s, 1H), 7.50 (dd, J = 8.9, 2.1 Hz, 3H), 7.19 (d, J = 8.3 Hz, 2H), 6.77 (d, J = 8.6 Hz, 1H), 6.56 (d, J = 2.1 Hz, 1H), 5.46-5.33 (m, 1H), 4.57-4.48 (m, 1H), 4.26-4.13 (m, 3H), 4.00-3.74 (m, 6H) , 2.92-2.83 (m, 1H), 2.22-2.12 (m, 1H), 2.10 (s, 3H), 1.88-1.79 (m, 2H), 1.70 (s, 2H), 1.63- 1.55 (m, 2H), 1.54- 1.41 (m, 12H). |
| 65 | 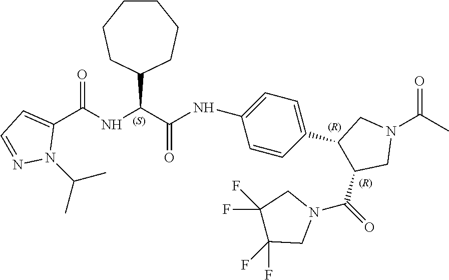 | 663.3 | ¹H NMR (400 MHz, Chloroform-d) δ 8.18 (s, 1H), 7.50 (dd, J = 8.9, 2.1 Hz, 3H), 7.19 (d, J = 8.3 Hz, 2H), 6.77 (d, J = 8.6 Hz, 1H), 6.56 (d, J = 2.1 Hz, 1H), 5.46-5.33 (m, 1H), 4.57-4.48 (m, 1H), 4.26-4.13 (m, 3H), 4.00-3.74 (m, 6H) , 2.92-2.83 (m, 1H), 2.22-2.12 (m, 1H), 2.10 (s, 3H), 1.88-1.79 (m, 2H), 1.70 (s, 2H), 1.63- 1.55 (m, 2H), 1.54- 1.41 (m, 12H). |
| 66 | 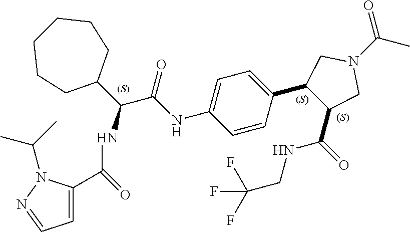 | 619.3 | ¹H NMR (400 MHz, Chloroform-d) δ 8.18 (s, 1H), 7.50 (dd, J = 8.9, 2.1 Hz, 3H), 7.19 (d, J = 8.3 Hz, 2H), 6.77 (d, J = 8.6 Hz, 1H), 6.56-6.51 (m, 2H), 5.46-5.33 (m, 1H), 4.57-4.48 (m, 1H), 4.26-4.13 (m, 3H), 4.00-3.74 (m, 2H), 3.55-3.52(m, 2H), 2.92-2.83 (m, 1H), 2.22-2.12 (m, 1H), |
| | | | 2.10 (s, 3H), 1.88- |
| | | | 1.79 (m, 5H), 1.70 (s, |
| | | | 2H), 1.63-1.55 (m, |
| | | | 2H), 1.54-1.41 (m, |
| | | | 12H). |
| 67 | 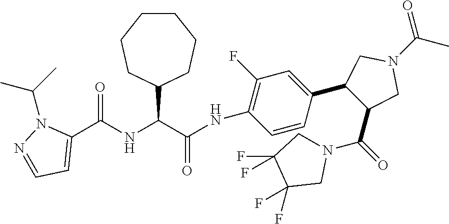 | 681.3 | ¹H NMR (400 MHz, Chloroform-d) δ 8.18 (s, 1H), 7.19-7.12(m, 2H), 6.77 (d, J = 8.6 Hz, 1H), 6.56 (d, J = 2.1 Hz, 1H), 5.46- 5.33 (m, 1H), 4.57- 4.48 (m, 1H), 4.26- 4.13 (m, 3H), 4.00- 3.74 (m, 6H) , 2.92- 2.83 (m, 1H), 2.22- 2.12 (m, 1H), 2.10 (s, 3H), 1.88-1.79 (m, 2H), 1.70 (s, 2H), 1.63- |
| | | | 1.55 (m, 2H), 1.54- |
| | | | 1.41 (m, 12H). |
| 68 | 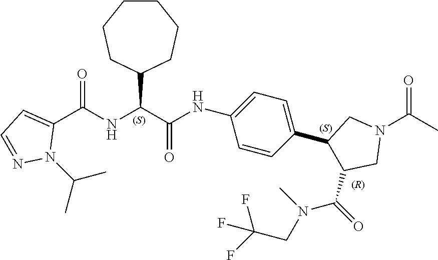 | 633.3 | ¹H NMR (400 MHz, Chloroform-d) δ 8.18 (s, 1H), 7.50 (dd, J = 8.9, 2.1 Hz, 3H), 7.19 (d, J = 8.3 Hz, 2H), 6.77 (d, J = 8.6 Hz, 1H), 6.56-6.51 (m, 1H), 5.46-5.33 (m, 1H), 4.57-4.48 (m, 1H), 4.26-4.13 (m, 3H), 4.00-3.74 (m, 2H), 3.55-3.52(m, 2H), 2.92-2.83 (m, 4H), 2.22-2.12 (m, 1H), 2.10 (s, 3H), 1.88- 1.79 (m, 5H), 1.70 (s, |
| | | | 2H), 1.63-1.55 (m, |
| | | | 2H), 1.54-1.41 (m, |
| | | | 12H). |

### Example 3 Evaluation of the In Vitro Bioactivity

| Materials and Reagents | Vendor | Cat# |
|---|---|---|
| HEK-Blue IL-17 | InvivoGen | hkb-il17 |
| Recombinant human IL-17A protein | R&D | 7955-IL |
| Reconstitution Buffer 2 (BSA/PBS) | R&D | RB02 |
| DMEM, high glucose | Gibco | 11965-092 |
| DMEM, High Glucose, HEPES, no | Gibco | 21063-029 |
| Phenol Red | | |
| FBS | AusGeneX | FBS500-S |
| Penicillin-Streptomycin(10000 | Gibco | 15140-122 |
| U/ml, 100 ml) | | |
| Normocin | InvivoGen | ant-nr-1 |
| HEK-Blue Selection | InvivoGen | hb-sel |
| DMSO | Sigma | D8418 |
| PBS, pH 7.4 | Gibco | 10010-031 |
| SEAPorter | Novus | NBP2-25285 |
| IL-17A Inhibitor-1 | MCE | HY-139206 |

| | | Cat# or |
|---|---|---|
| Consumables and Instrument | Vendor | Model |
| CulturPlate-384, White Opaque 384- | PerkinElmer | 6007680-50 |
| well Microplate | | |
| Tissue Culture (TC)-treated surface, | Corning | CLS3764 |
| black polystyrene, flat bottom | | |
| Echo Qualified 384-Well Polypro- | LabCtye | PP-0200 |
| pylene Microplate, Clear, Sterile | | |
| Reagent reservoirs, sterilized | JetBioFil | LTT-001-50 |
| Biological Safety Cabinet(Class II) | Thermo Scientific | 1389 |
| Forma CO₂ Incubator | Thermo Scientific | 371 |
| Countess 3 automated cell counter | Invitrogen | A49891 |
| Plate shaker | Thermo | 4625- |
| | | 1CECN/THZQ |
| Centrifuge | Eppendorf | 5810R |
| Echo 655 | Labcyte | 655 |
| Envision 2105 multilabel Reader | PerkinElmer | Envision |
| | | 2105 |
| Votexer | IKA | MS3 digital |

### Compound Management

1. The test Compounds were 3-fold serial diluted from 10 mM for 10 doses in DMSO.

2. Prepare Reference Compounds (IL-17A-Inhibitor-1) were 3-fold serial diluted from 1 mM for 10 doses in DMSO.

3. Prepare 1000× Positive control (1 mM IL-17A-Inhibitor-1) and 1000× vehicle control (100% DMSO).

### Assay Procedure

a) Cells were cultured as the HEK-Blue™ IL-17 technical data sheet recommended. HEK-Blue IL-17 cells were assayed in the exponential growth phase.
b) Aspirate growth medium and rinse cells twice with PBS to remove phenol red.
c) Resuspend cells in test medium to a proper concentration.
d) Only cells with viability greater than 90% were used for assays.
e) Transfer 25nl of compound dilutions into 384-well assay plates using Echo655;
f) Seed 25 ul of HEK-Blue IL-17 cells at 8,000 cells/well into 384-well plate with rhIL-17A/A.
g) Cells were incubated for 20 hours at 37° C. under 5% CO₂ atmosphere.
h) Transfer 2 ul of cells supernatant into per well of 384-well assay plates.
i) Add 20 ul of PNPP substrate.
j) Measure the Absorbance at 405 nm on Envision 2105 plate reader.

### Data Analysis

RLU signal (LUMempd) is calculated for each well.
% Inhibition is calculated as follow:

$$\% inhibition = 100 - ( {Signal}_{cmpd} - {Signal}_{Ave \_ PC} ) / {Signal}_{Ave \_ VC} - {Signal}_{Ave \_ PC} ) \times 100.$$

Signalave_pc: The average signal for the positive controls across the plate.

Signalave_vc: The average signal for negative controls across the plate.

Calculate IC₅₀ and Plot effect-dose curve of cmpds:

Calculate IC₅₀ by fitting % Inhibition values and log of compound concentrations to nonlinear regression (dose response−variable slope) with Graphpad 8.0.

The results are summarized in Table 2

TABLE 2
The relative biological activity of
compounds in HEK-Bule cell Assay

| Example | IC₅₀ (nM) |
|---|---|
| 1 | 15.32 |
| 2 | 109.9 |
| 6 | 68.97 |
| 17 | 943.6 |
| 46 | 6038 |
| 49 | 1860 |
| 50 | 419.7 |
| 51 | 118 |
| 52 | 103 |
| 53 | 531.8 |
| 54 | 28.6 |
| 55 | 943.6 |
| 56 | 1454 |
| 57 | 46.8 |
| 58 | 39.3 |
| 59 | 142.8 |
| 60 | 315.3 |
| 61 | 66.79 |
| Cis-62 | 39.65 |
| 63 | 131.7 |
| 64 | 11.46 |
| 65 | 57.16 |
| 66 | 44.4 |
| 67 | 31.4 |
| 68 | 48.56 |

1. A compound of Formula I: 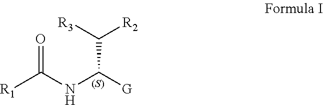
pharmaceutically acceptable salts thereof, deuterium substitutes thereof, and isomers thereof,
wherein
R₁ is selected from the group consisting of aryl, 5-6 membered heteroaryl, —C₁₋₃ alkyl-aryl and —C₁₋₃ alkyl-5-6 membered heteroaryl; wherein R₁ is optionally substituted with one or more R₁ₐ;
wherein R₁ₐ is selected from the group consisting of halo, oxo, —OH, —CN, —C₁₋₆ alkyl, and —C₀₋₂ alkyl-C₃₋₆ cycloalkyl; wherein each R₁ₐ is optionally substituted with one or more substituents each independently selected from halo;
wherein each of R₂ and R₃ is independently selected from the group consisting of H, —C₁₋₆ alkyl, aryl, C₃₋₁₀ cycloalkyl, —C₀₋₂ alkyl-C₃₋₁₀ cycloalkyl, 5-10 membered heterocyclyl, —C₀₋₂ alkyl-C₅₋₁₀ aryl and —C₀₋₂ alkyl-5-10 membered heterocyclyl; or R₂ and R₃ are combined with atoms to which they are attached to form a C₃₋₁₀ cycloalkyl or 5-10 membered heterocyclyl; wherein the C₃₋₁₀ cycloalkyl or the 5-10 membered heterocyclyl is optionally substituted with one or more substituents each independently selected from the group consisting of halo, —C₁₋₉ alkyl, and —C₁₋₈ haloalkyl;
wherein G is selected from the group consisting of 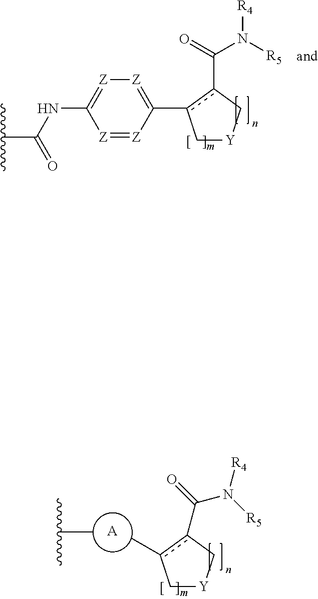
wherein m=0, 1, or 2;
n=0, 1, or 2;
wherein Z is N or —C(R₇)—;
wherein ring A is 9-10 membered heteroaryl; wherein ring A is optionally substituted with one or more substituents each independently selected from the group consisting of halogen, oxo, —OH, —CN, —C₁₋₆ alkyl, —C₁₋₆ alkoxy, —C₁₋₆ haloalkoxy, —C₃₋₁₀ cycloalkyl, heterocyclyl, —C₆₋₁₀ aryl and 5-10 membered heteroaryl;
wherein Y is selected from the group consisting of O, S, SO, S(O)₂, and 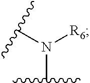
wherein each of R₄ and R₅ is independently selected from the group consisting of H, OH, —C₁₋₉ alkyl, —C₁₋₈ haloalkyl, —C₂₋₆ alkenyl, —C₂₋₆ alkynyl, —COR₆ₐ, —C₁₋₄ alkyl-C₃₋₈ cycloalkyl, —C₁₋₄ alkyl-heterocyclyl, —C₃₋₁₀ cycloalkyl, heterocyclyl, —C₆₋₁₀ aryl, 5-10 membered heteroaryl, —C₁₋₄ alkyl-SO₂—C₁₋₄ alkyl, and —C₁₋₄ alkyl-SO₂—C₃₋₈ cycloalkyl;
wherein each of R₄ and R₅ is optionally substituted with one or more substituents each independently selected from the group consisting of H, halogen, oxo, —OH, —CN, —C₁₋₆ alkyl, —C₁₋₆ alkoxy, —C₁₋₆ haloalkoxy, —C₃₋₁₀ cycloalkyl, heterocyclyl, —C₆₋₁₀ aryl, and 5-10 membered heteroaryl; or R₄ and R₅ are combined with atoms to which they are attached to form 3-10 membered heterocyclyl; wherein the 3-10 membered heterocyclyl is optionally substituted with one or more substituents each independently selected from the group consisting of halo, —C₁₋₉ alkyl, and —C₁₋₈ haloalkyl;
wherein R₆ is independently selected from the group consisting of H, C₁₋₉ alkyl, C₁₋₈ haloalkyl, —C₁₋₆ alkyl-alkoxy, —C₁₋₆ alkyl-cycloalkoxy, C₂₋₆ alkenyl, C₂₋₆ alkynyl, C₃₋₁₀ cycloalkyl, heterocyclyl, C₆₋₁₀ aryl, 5-10 membered heteroaryl, CO₂R₆ₐ, COR₆ₐ, CON(R₆ₐ)(R_{6b}), —S(O) R₆ₐ, —S(O)(NH)R₆ₐ, —S(O)₂R₆ₐ, —S(O)₂OR₆ₐ—S(O)₂N(R₆ₐ)(R_{6b}), and —S(O)(NR₆ₐ)R₆ₐ;
wherein R₆ is optionally substituted with one or more substituents each independently selected from the group consisting of halogen, oxo, —OH, —CN, —C₁₋₆ alkyl, —C₁₋₆ alkoxy, —C₁₋₆ haloalkoxy, —C₁₋₆ haloalkyl, —C₃₋₁₀ cycloalkyl, heterocyclyl, —C₆₋₁₀ aryl, and 5-10 membered heteroaryl;
wherein each of R₆ₐ and R_{6b} is independently selected from the group consisting of H, C₁₋₉ alkyl, C₁₋₈ haloalkyl, C₂₋₆ alkenyl, C₂₋₆ alkynyl, C₃₋₁₀ cycloalkyl, heterocyclyl, C₆₋₁₀ aryl, and 5-10 membered heteroaryl; or R₆ₐ and R_{6b} are combined with atoms to which they are attached to form a C₃₋₁₀ cycloalkyl or 5-10 membered heterocyclyl; wherein the C₃₋₁₀ cycloalkyl or 5-10 membered heterocyclyl is optionally substituted with one or more substituents each independently selected from the group consisting of halo, —C₁₋₉ alkyl, and —C₁₋₈ haloalkyl;
wherein each of R₆ₐ and R_{6b} is optionally substituted with one or more substituents each independently selected from the group consisting of halogen, oxo, —OH, —CN, —C₁₋₆ alkyl, —C₁₋₆ alkoxy, —C₁₋₆ haloalkoxy, —C₁₋₆ haloalkyl, —C₃₋₁₀ cycloalkyl, heterocyclyl, —C₆₋₁₀ aryl, 5-10 membered heteroaryl, —C₁₋₄ alkyl-C₃₋₈ cycloalkyl, and —C₁₋₄ alkyl-heterocyclyl;
wherein R₇ is selected from the group consisting of H, halogen, oxo, —OH, —CN, —C₁₋₉ alkyl, —C₁₋₈ haloalkyl, —C₁₋₆ alkoxy, —C₁₋₆ haloalkoxy, —C₂₋₆ alkenyl, —C₂₋₆ alkynyl, —CO₂R₇ₐ, —COR₇ₐ, —CONHR₇ₐ, —NO₂, —NH₂, —N₃, —SH, —O(C₁₋₉ alkyl), —O(C₁₋₈ haloalkyl), —NH(C₁₋₉ alkyl), —NH(C₁₋₈ haloalkyl), —N(C₁₋₉ alkyl)₂, and —N(C₁₋₈ haloalkyl)₂;
wherein R₇ₐ is selected from the group consisting of H, —C₁₋₆ alkyl, —C₁₋₆ haloalkyl, —C₁₋₄ alkyl-C₃₋₈ cycloalkyl, —C₁₋₄ alkyl-heterocyclyl, C₃₋₁₀ cycloalkyl, heterocyclyl, C₆₋₁₀ aryl, and 5-10 membered heteroaryl; and wherein the 5-10 membered heteroaryl or the heterocyclyl is optionally substituted with one or more substituents each independently selected from the group consisting of N, O, and S.

2. The compound of claim 1, pharmaceutically acceptable salts thereof, deuterium substitutes thereof, and isomers thereof, wherein the compound has a structure of Formula Ia or Ib:

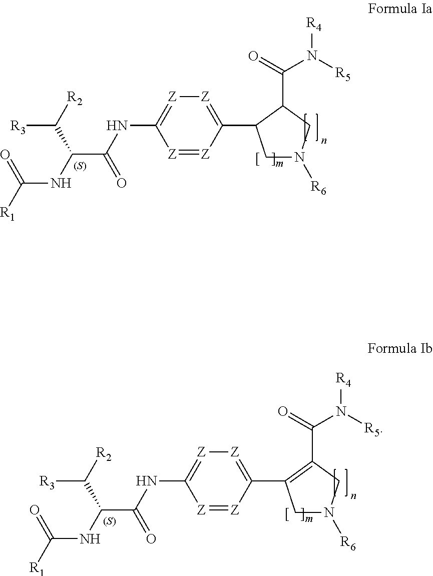

3. The compound of claim 1, pharmaceutically acceptable salts thereof, deuterium substitutes thereof, isomers thereof, wherein the compound has a structure of Formula IIa or IIb:

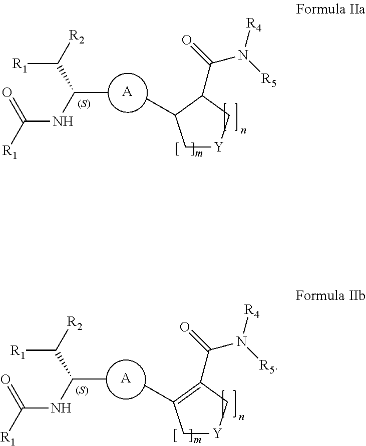

4. The compound of claim 1, pharmaceutically acceptable salts thereof, deuterium substitutes thereof, isomers thereof, wherein ring A is selected from the group consisting of:

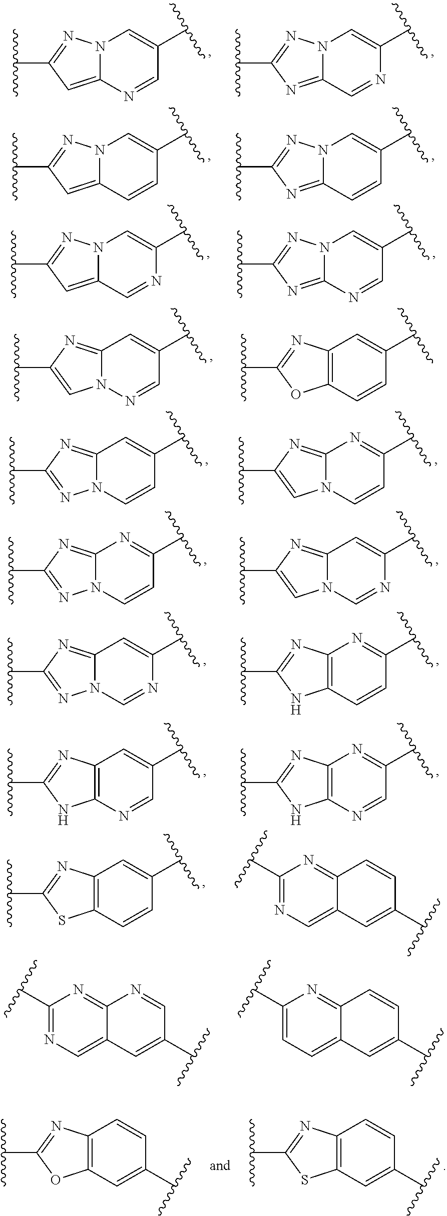

5. The compound of claim 1, pharmaceutically acceptable salts thereof, deuterium substitutes thereof, isomers thereof, wherein the compound has a structure of Formula III: 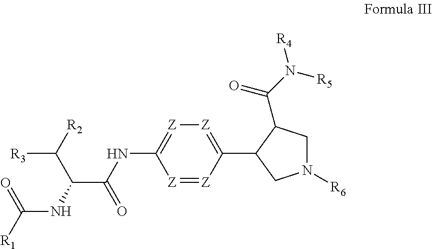
wherein R₁ is selected from the group consisting of aryl, 5-6 membered heteroaryl; wherein R₁ is optionally substituted with one or more R₁ₐ; wherein each of R₁ₐ is independently selected from the group consisting of halo-C₁₋₆ alkyl, and —C₀₋₂ alkyl-C₃₋₆ cycloalkyl; wherein each R₁ₐ is optionally substituted with one or more substituents each independently selected from halo;
wherein each of R₄ and R₅ is independently selected from the group consisting of H, OH, —C₁₋₉ alkyl, —C₁₋₈ haloalkyl, —C₂₋₆ alkenyl, —C₂₋₆ alkynyl, —COR₆ₐ, —C₁₋₄ alkyl-C₃₋₈ cycloalkyl, —C₁₋₄ alkyl-heterocyclyl, —C₃₋₁₀ cycloalkyl, heterocyclyl, —C₆₋₁₀ aryl, 5-10 membered heteroaryl, —C₁₋₄ alkyl-SO₂—C₁₋₄ alkyl, and —C₁₋₄ alkyl-SO₂—C₃₋₈ cycloalkyl;
wherein each of R₄ and R₅ is optionally substituted with one or more substituents each independently selected from the group consisting of H, halogen, oxo, —OH, —CN, —C₁₋₆ alkyl, —C₁₋₆ alkoxy, —C₁₋₆ haloalkoxy, —C₃₋₁₀ cycloalkyl, heterocyclyl, —C₆₋₁₀ aryl, and 5-10 membered heteroaryl; or R₄ and R₅ are combined with atoms to which they are attached to form 3-10 membered heterocyclyl; wherein the 3-10 membered heterocyclyl is optionally substituted with one or more substituents each independently selected from the group consisting of halo, —C₁₋₉ alkyl, and —C₁₋₈ haloalkyl;
wherein R₆ is independently selected from the group consisting of H, C₁₋₉ alkyl, C₁₋₈ haloalkyl, —C₁₋₆ alkyl-alkoxy, —C₁₋₆ alkyl-cycloalkoxy, C₃₋₁₀ cycloalkyl, heterocyclyl, CO₂R₆ₐ, COR₆ₐ, CON(R₆ₐ)(R_{6b});
wherein R₆ is optionally substituted with one or more substituents each independently selected from the group consisting of halogen, —C₁₋₆ alkyl, —C₁₋₆ alkoxy, —C₁₋₆ haloalkoxy, —C₁₋₆ haloalkyl, —C₃₋₁₀ cycloalkyl.

6. The compound of claim 1, wherein each of R₄ and R₅ is independently selected from the group consisting of H, —C₁₋₉ alkyl, —C₁₋₈ haloalkyl, —C₁₋₄ alkyl-C₃₋₈ cycloalkyl, —C₁₋₄ alkyl-heterocyclyl, and —C₃₋₁₀ cycloalkyl;
or R₄ and R₅ are combined with atoms to which they are attached to form 3-10 membered heterocyclyl; wherein the 3-10 membered heterocyclyl is optionally substituted with one or more substituents each independently selected from the group consisting of halo, —C₁₋₉ alkyl, and —C₁₋₈ haloalkyl.

7. The compound of claim 1, wherein R₆ is independently selected from the group consisting of H, C₁₋₉ alkyl, C₁₋₈ haloalkyl, —C₁₋₆ alkyl-alkoxy, —C₁₋₆ alkyl-cycloalkoxy, C₃₋₁₀ cycloalkyl, heterocyclyl, CO₂R₆ₐ, COR₆ₐ, and CON(R₆ₐ)(R_{6b});
wherein R₆ is optionally substituted with one or more substituents each independently selected from the group consisting of halogen, —C₁₋₆ alkyl, —C₁₋₆ alkoxy, —C₁₋₆ haloalkoxy, —C₁₋₆ haloalkyl, and —C₃₋₁₀ cycloalkyl.

8. The compound of claim 1, wherein each of R₆ₐ and R_{6b} is independently selected from the group consisting of H, C₁₋₉ alkyl, C₁₋₈ haloalkyl, C₃₋₁₀ cycloalkyl, and heterocyclyl; or R₆ₐ and R_{6b} are combined with atoms to which they are attached to form a C₃₋₁₀ cycloalkyl or 5-10 membered heterocyclyl; wherein the C₃₋₁₀ cycloalkyl or 5-10 membered heterocyclyl is optionally substituted with one or more substituents each independently selected from the group consisting of halo, —C₁₋₉ alkyl, and —C₁₋₈ haloalkyl;
wherein each of R₆ₐ and R_{6b} is optionally substituted with one or more substituents each independently selected from the group consisting of halogen, —C₁₋₆ alkyl, —C₁₋₆ alkoxy, —C₁₋₆ haloalkoxy, —C₁₋₆ haloalkyl, —C₃₋₁₀ cycloalkyl, and heterocyclyl.

9. The compound of claim 1, wherein R₁ is selected from the group consisting of group of

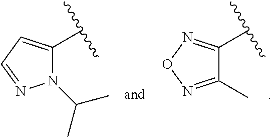

10. The compound of claim 1, wherein R₂ and R₃ is independently selected from the group consisting of H, —C₁₋₆ alkyl, C₃₋₁₀ cycloalkyl, and —C₀₋₂ alkyl-C₃₋₁₀ cycloalkyl; or R₂ and R₃ are combined with atoms to which they are attached to form a C₃₋₁₀ cycloalkyl; wherein the C₃₋₁₀ cycloalkyl is optionally substituted with one or more halo.

11. A compound, pharmaceutically acceptable salts thereof, deuterium substitutes thereof, and isomers thereof, wherein the compound is selected from the group consisting of

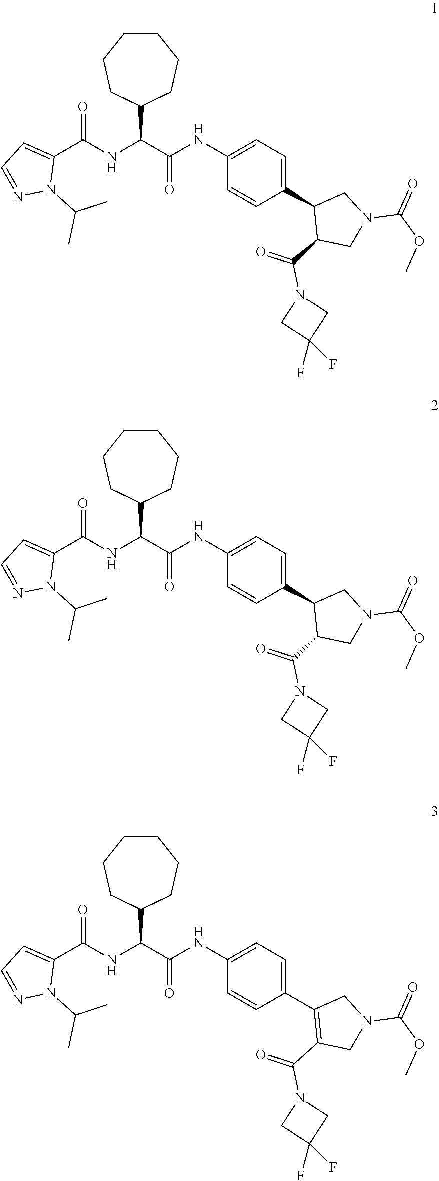 [SMILES: COC(=O)N1C[C@H](c2ccc(NC(=O)[C@@H](NC(=O)c3ccnn3C(C)C)C3CCCCCC3)cc2)[C@H](C(=O)N2CC(F)(F)C2)C1 ; COC(=O)N1C[C@H](C(=O)N2CC(F)(F)C2)[C@@H](c2ccc(NC(=O)[C@@H](NC(=O)c3ccnn3C(C)C)C3CCCCCC3)cc2)C1 ; COC(=O)N1CC(C(=O)N2CC(F)(F)C2)=C(c2ccc(NC(=O)[C@@H](NC(=O)c3ccnn3C(C)C)C3CCCCCC3)cc2)C1]

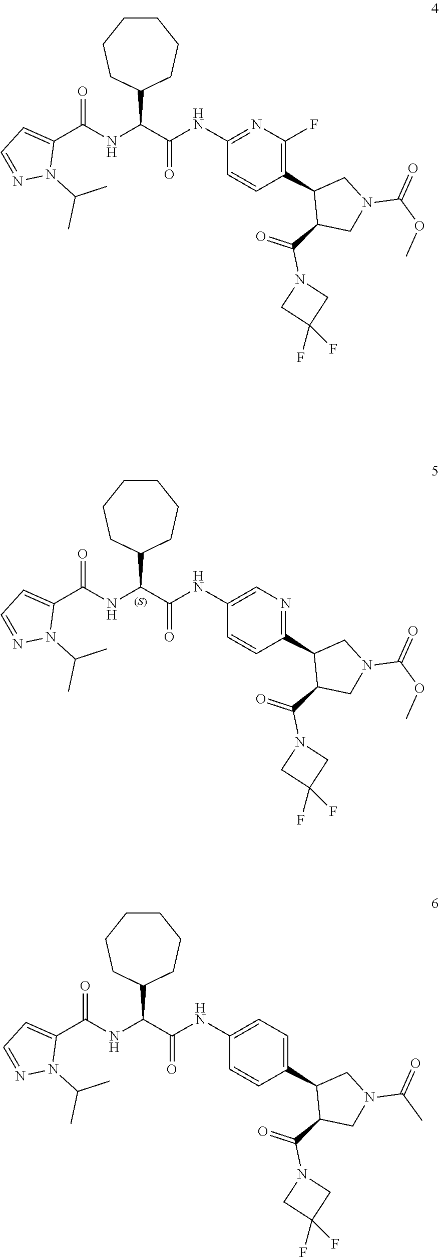

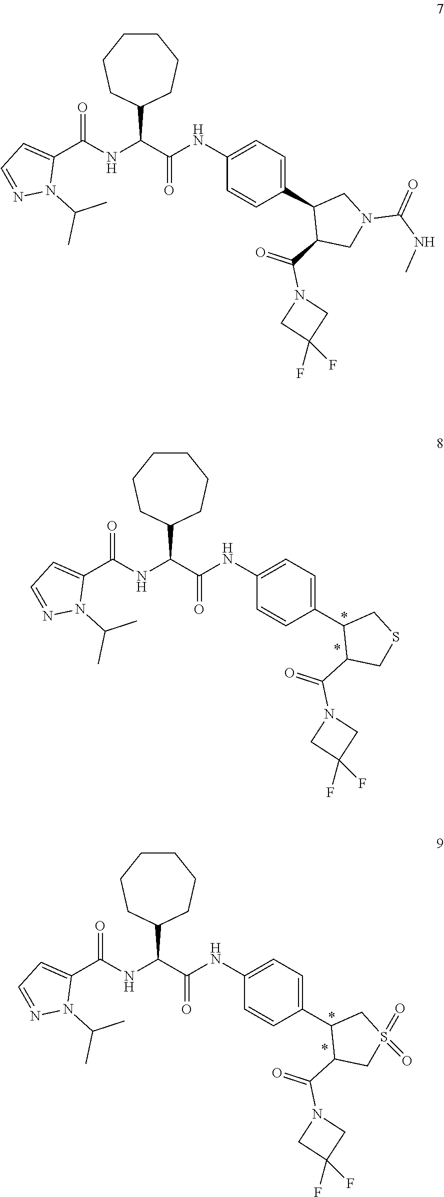 [SMILES: CNC(=O)N1C[C@H](c2ccc(NC(=O)[C@@H](NC(=O)c3ccnn3C(C)C)C3CCCCCC3)cc2)[C@H](C(=O)N2CC(F)(F)C2)C1 ; CC(C)n1nccc1C(=O)N[C@H](C(=O)Nc1ccc(C2CSCC2C(=O)N2CC(F)(F)C2)cc1)C1CCCCCC1 ; CC(C)n1nccc1C(=O)N[C@H](C(=O)Nc1ccc(C2CS(=O)(=O)CC2C(=O)N2CC(F)(F)C2)cc1)C1CCCCCC1]

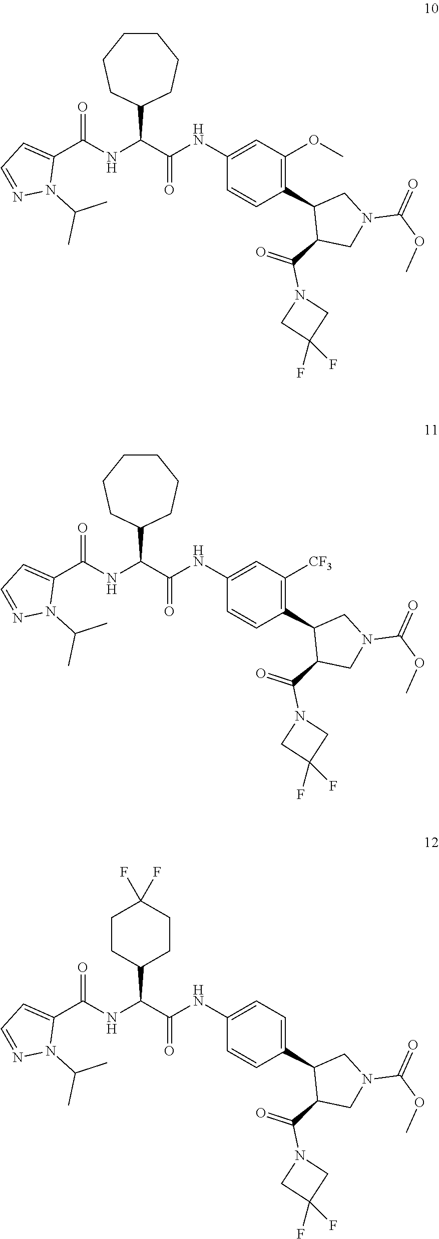 [SMILES: COC(=O)N1C[C@H](c2ccc(NC(=O)[C@@H](NC(=O)c3ccnn3C(C)C)C3CCCCCC3)cc2OC)[C@H](C(=O)N2CC(F)(F)C2)C1 ; COC(=O)N1C[C@H](c2ccc(NC(=O)[C@@H](NC(=O)c3ccnn3C(C)C)C3CCCCCC3)cc2C(F)(F)F)[C@H](C(=O)N2CC(F)(F)C2)C1 ; COC(=O)N1C[C@H](c2ccc(NC(=O)[C@@H](NC(=O)c3ccnn3C(C)C)C3CCC(F)(F)CC3)cc2)[C@H](C(=O)N2CC(F)(F)C2)C1]

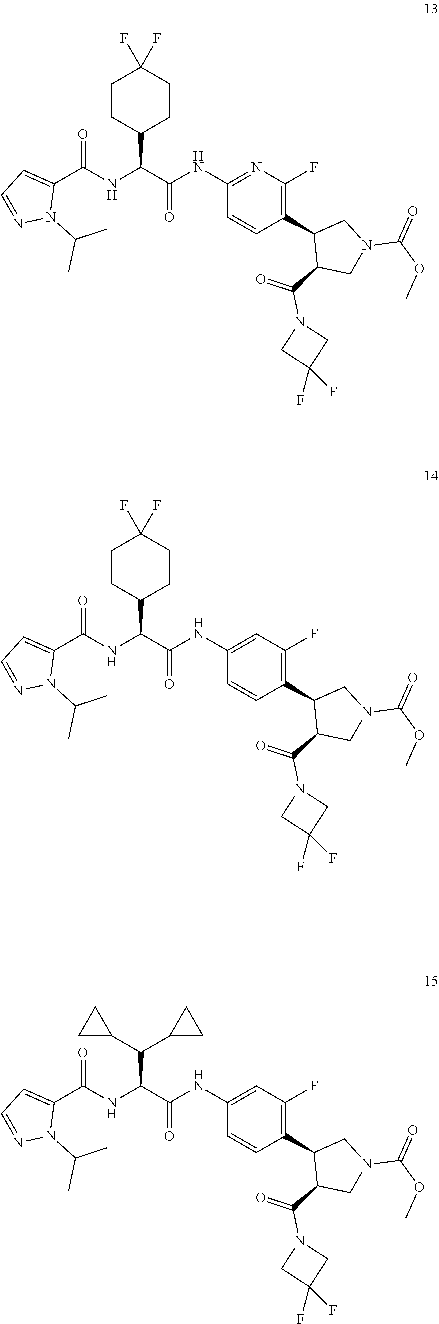 [SMILES: COC(=O)N1C[C@H](c2ccc(NC(=O)[C@@H](NC(=O)c3ccnn3C(C)C)C3CCC(F)(F)CC3)nc2F)[C@H](C(=O)N2CC(F)(F)C2)C1 ; COC(=O)N1C[C@H](c2ccc(NC(=O)[C@@H](NC(=O)c3ccnn3C(C)C)C3CCC(F)(F)CC3)cc2F)[C@H](C(=O)N2CC(F)(F)C2)C1 ; COC(=O)N1C[C@H](c2ccc(NC(=O)[C@@H](NC(=O)c3ccnn3C(C)C)C(C3CC3)C3CC3)cc2F)[C@H](C(=O)N2CC(F)(F)C2)C1]

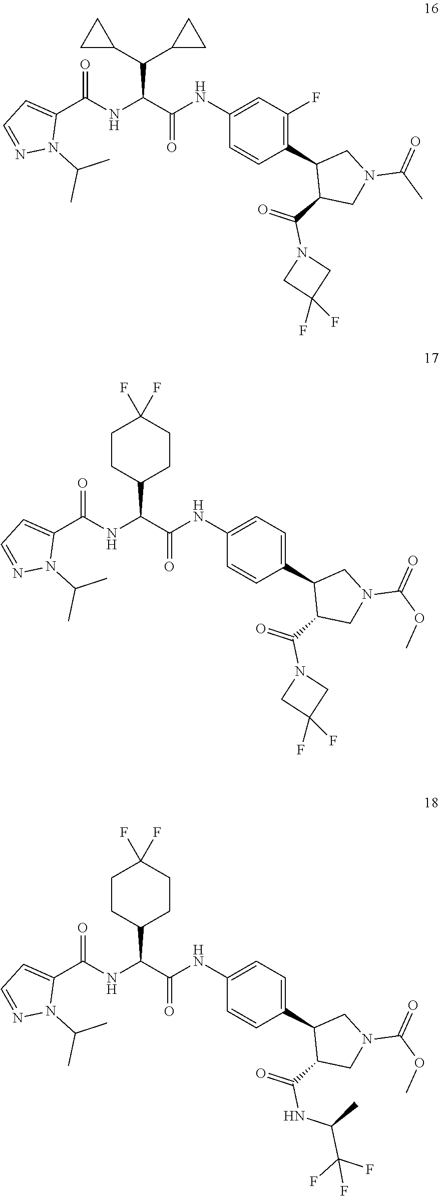 [SMILES: CC(=O)N1C[C@H](c2ccc(NC(=O)[C@@H](NC(=O)c3ccnn3C(C)C)C(C3CC3)C3CC3)cc2F)[C@H](C(=O)N2CC(F)(F)C2)C1 ; COC(=O)N1C[C@H](C(=O)N2CC(F)(F)C2)[C@@H](c2ccc(NC(=O)[C@@H](NC(=O)c3ccnn3C(C)C)C3CCC(F)(F)CC3)cc2)C1 ; COC(=O)N1C[C@H](C(=O)N[C@@H](C)C(F)(F)F)[C@@H](c2ccc(NC(=O)[C@@H](NC(=O)c3ccnn3C(C)C)C3CCC(F)(F)CC3)cc2)C1]

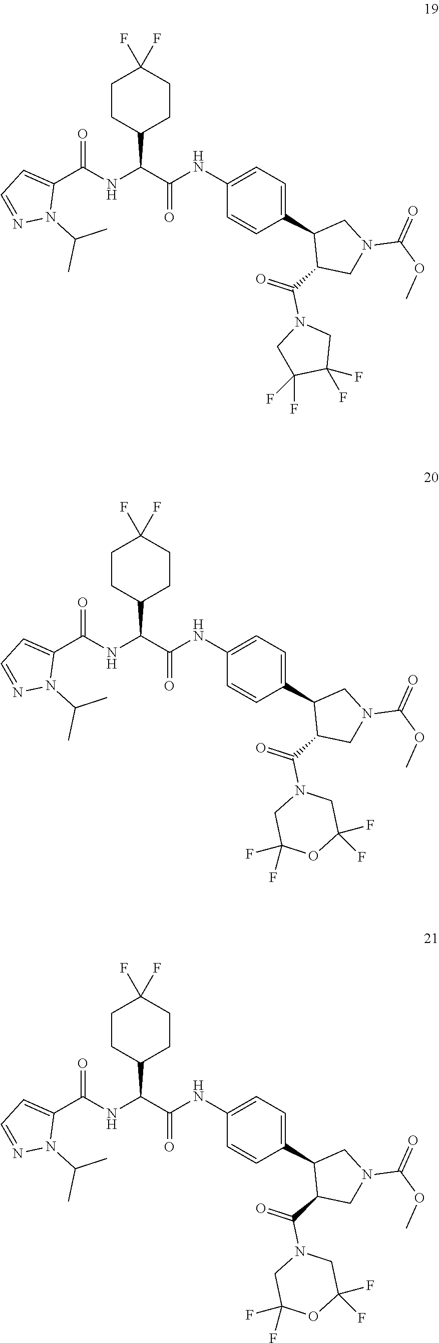 [SMILES: COC(=O)N1C[C@H](C(=O)N2CC(F)(F)C(F)(F)C2)[C@@H](c2ccc(NC(=O)[C@@H](NC(=O)c3ccnn3C(C)C)C3CCC(F)(F)CC3)cc2)C1 ; COC(=O)N1C[C@H](C(=O)N2CC(F)(F)OC(F)(F)C2)[C@@H](c2ccc(NC(=O)[C@@H](NC(=O)c3ccnn3C(C)C)C3CCC(F)(F)CC3)cc2)C1 ; COC(=O)N1C[C@H](c2ccc(NC(=O)[C@@H](NC(=O)c3ccnn3C(C)C)C3CCC(F)(F)CC3)cc2)[C@H](C(=O)N2CC(F)(F)OC(F)(F)C2)C1]

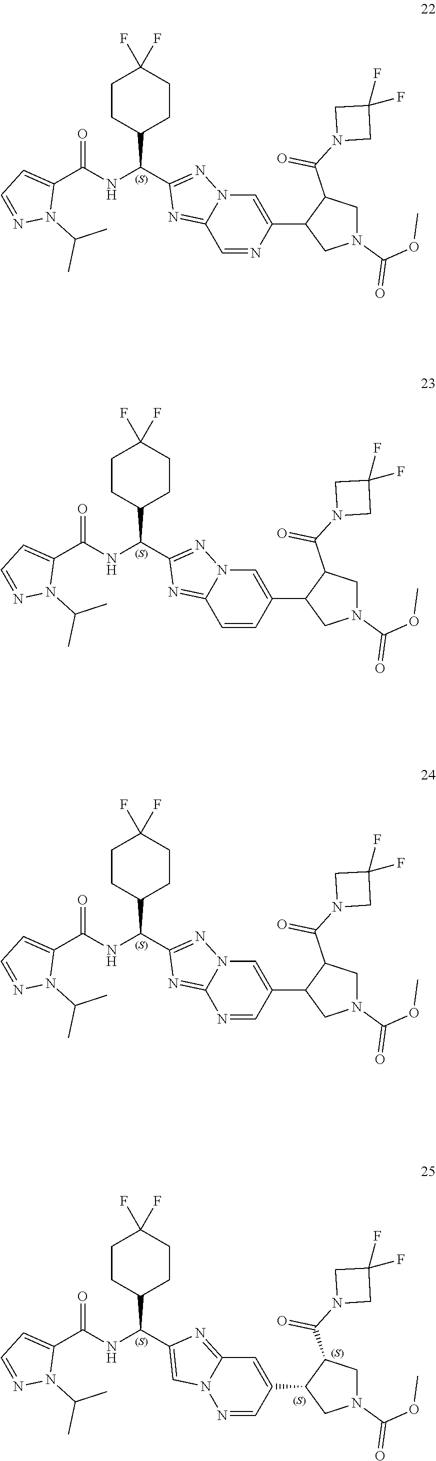

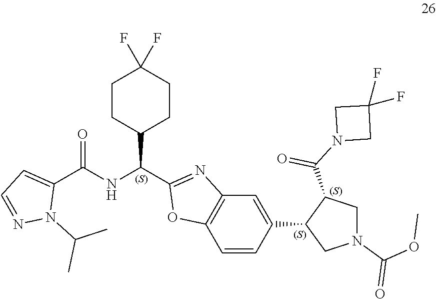

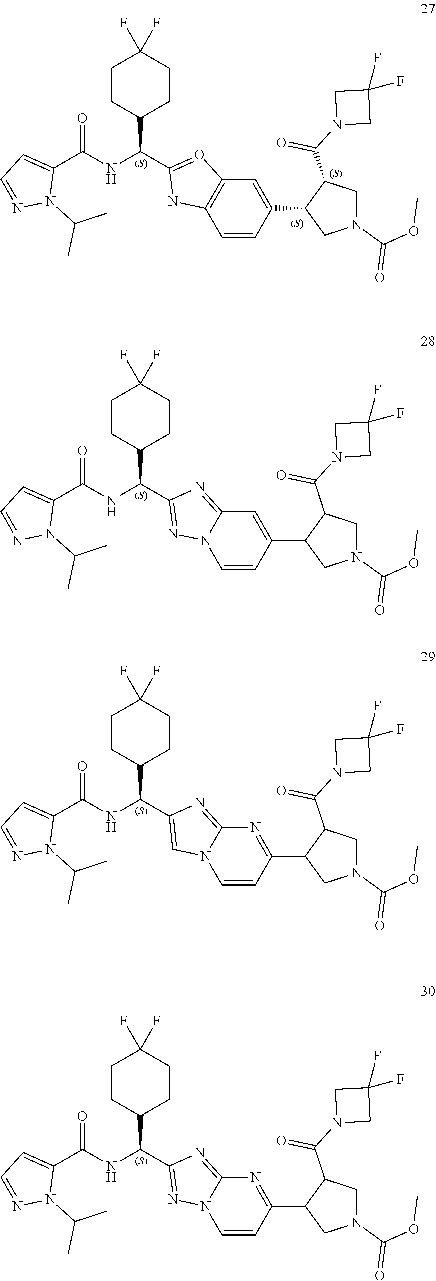

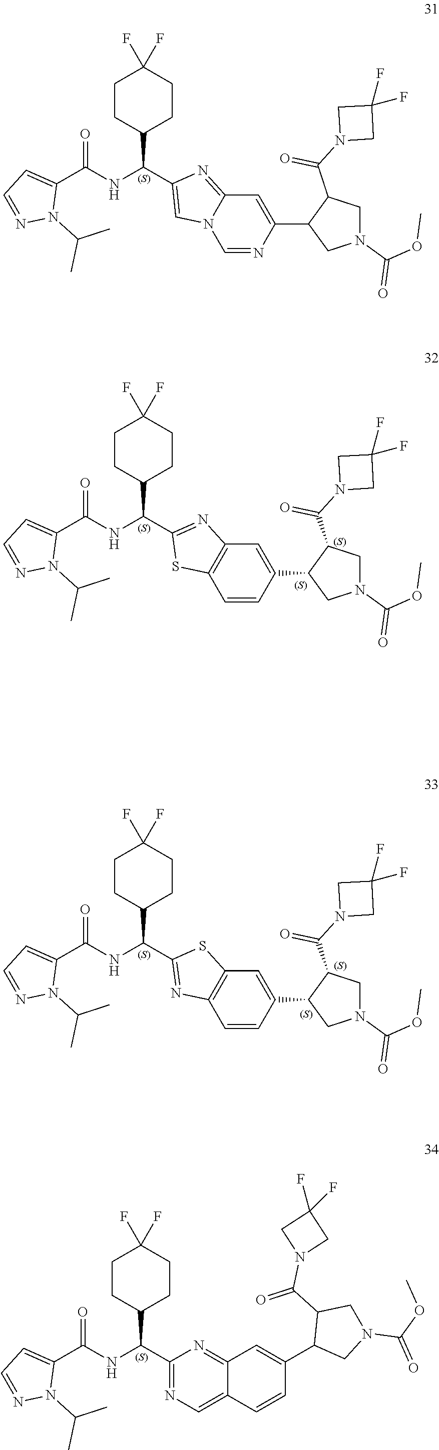

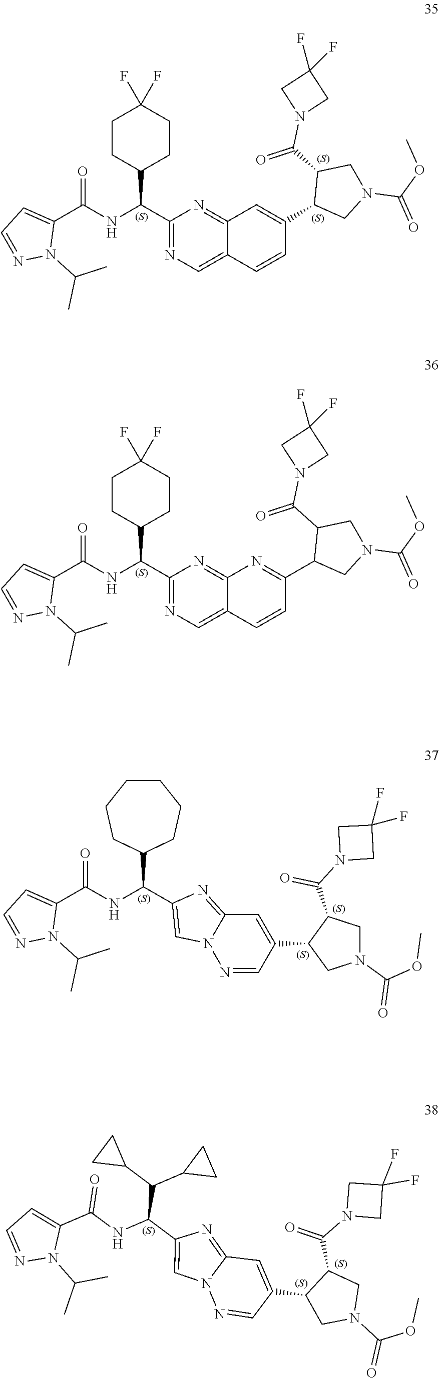

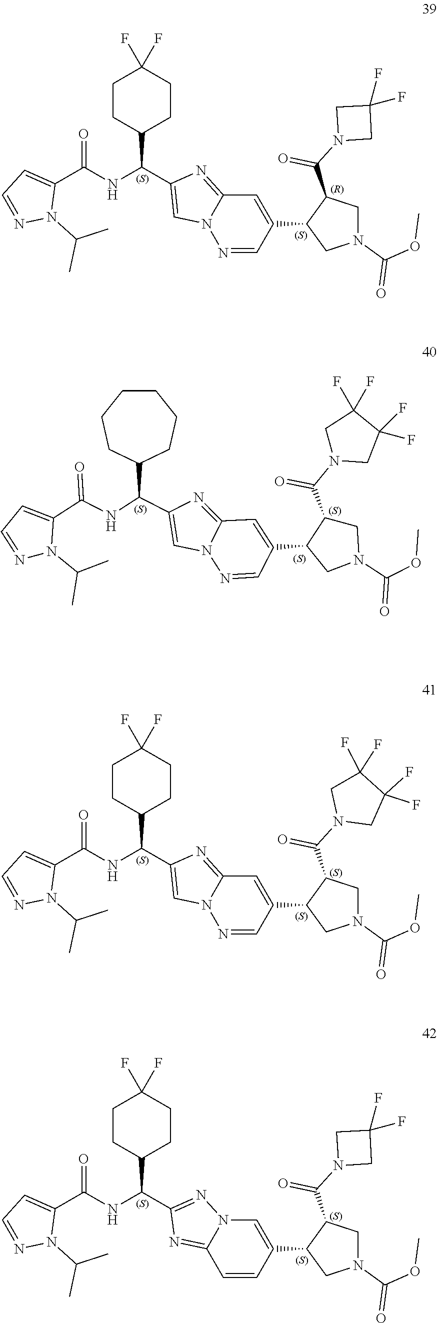

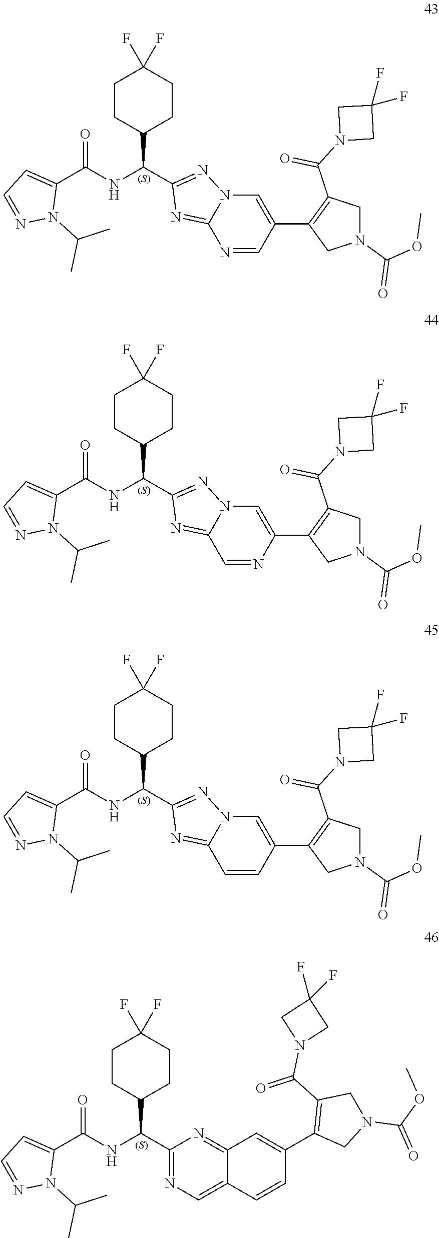

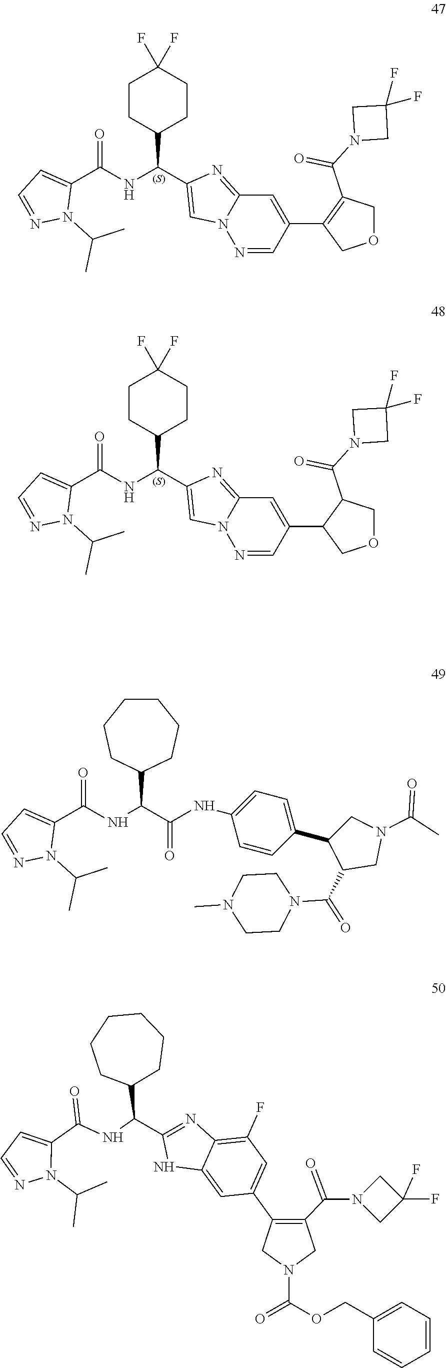

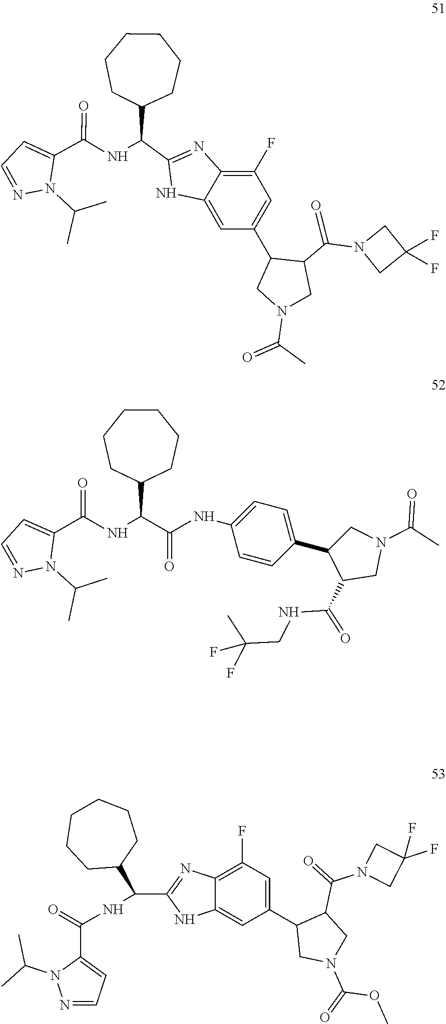

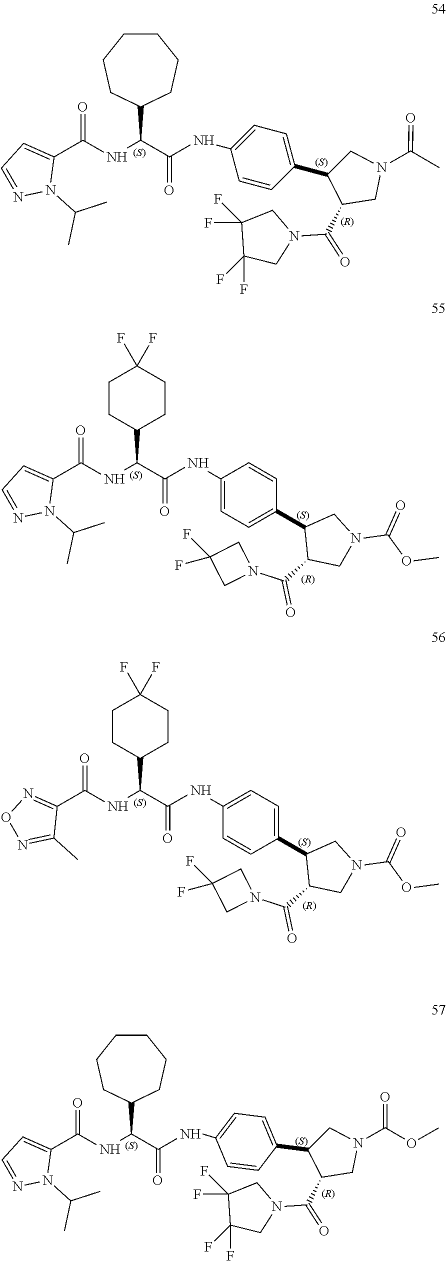

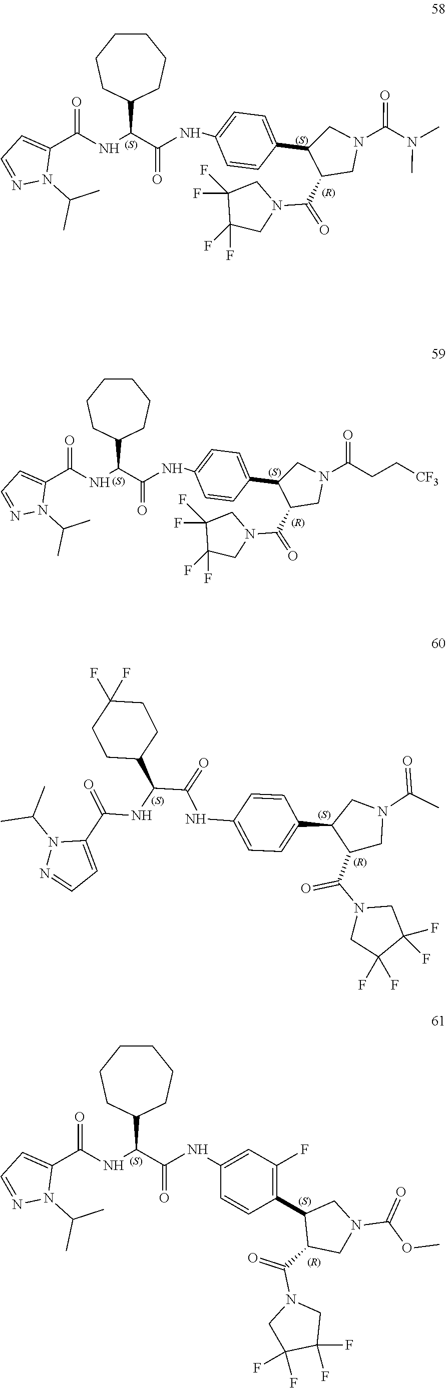

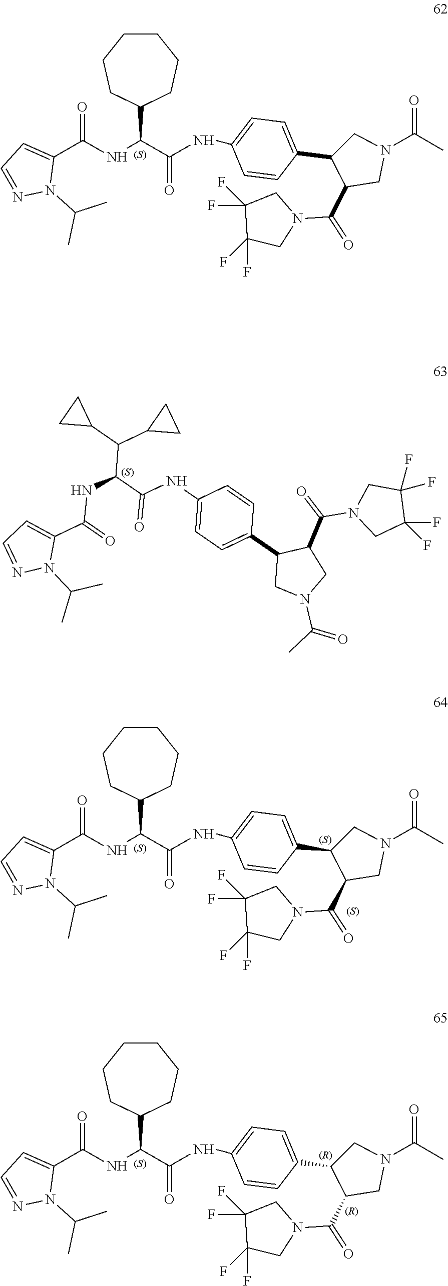

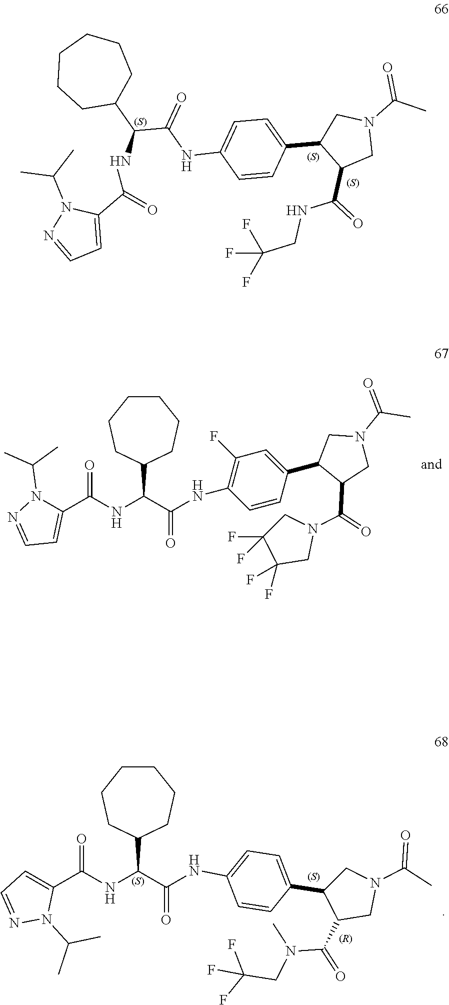

12. A pharmaceutical composition, comprising: the compound of claim 1, and a pharmaceutically acceptable excipient.

13. A method of modulating IL-17A, comprising administering to a subject in need thereof an effective amount of the compound of claim 1, a pharmaceutically acceptable salt thereof, a deuterium substitute thereof, or an isomer thereof.

14. A method of treating an inflammatory disease or condition, comprising administering to a subject in need thereof an effective amount of the compound of claim 1, a pharmaceutically acceptable salt thereof, a deuterium substitute thereof, or an isomer thereof.

15. The method of claim 14, wherein the inflammatory disease or condition is selected from the group consisting of plaque psoriasis, guttate psoriasis, inverse psoriasis, pustular psoriasis, erythrodermic psoriasis, aspsoriatic arthritis, Palmoplantar psoriasis, Non-infectious uveitis, psoriasis, rheumatoid arthritis, spondyloarthritis, multiple sclerosis, psoriatic arthritis, axial spondyloarthritis, ankylosing spondylitis, hidradenitis suppurativa, systemic lupus erythematosus, palmoplantar pustulosis (PPP), atopic dermatitis, asthma, and/or COPD.
